(19) United States
(12) Patent Application Publication
(10) Pub. No.: US 2023/0357798 A1
(43) Pub. Date: Nov. 9, 2023

(54) GENE CORRECTION FOR X-CGD IN HEMATOPOIETIC STEM AND PROGENITOR CELLS

(71) Applicant: THE BOARD OF TRUSTEES OF THE LELAND STANFORD JUNIOR UNIVERSITY, Stanford, CA (US)

(72) Inventors: Mara Pavel-Dinu, Stanford, CA (US); Matthew H. Porteus, Stanford, CA (US)

(73) Assignee: The Board of Trustees of the Leland Stanford Junior University, Stanford, CA (US)

(21) Appl. No.: 18/029,798

(22) PCT Filed: Oct. 12, 2021
(86) PCT No.: PCT/US2021/054595

§ 371 Date: Mar. 31, 2023

Related U.S. Application Data
(60) Provisional application No. 63/090,679, filed on Oct. 12, 2020.

(51) Int. Cl. C12N 15/90; C12N 9/22; C12N 15/11; A61K 35/28; A61P 37/00

(52) U.S. Cl. CPC C12N 15/907; C12N 9/22; C12N 15/11; C12N 15/111; A61K 35/28; A61P 37/00; C12N 2310/20; C12N 2310/321; C12N 2310/315; C12N 2320/34

(57) ABSTRACT

The present disclosure provides methods and compositions for treating X-CGD in subjects, comprising genetically modifying cells from the subjects ex vivo by integrating a functional, codon-optimized CYBB cDNA at the endogenous CYBB locus.

### CROSS REFERENCE TO RELATED APPLICATIONS

The present application claims priority to U.S. Provisional Pat. Appl. No. 63/090,679, filed on Oct. 12, 2020, which application is incorporated herein by reference in its entirety.

### STATEMENT AS TO RIGHTS TO INVENTIONS MADE UNDER FEDERALLY SPONSORED RESEARCH AND DEVELOPMENT

This invention was made with government support under Grant No. R01 AI097320-01 awarded by the National Institutes of Health. The government has certain rights in the invention.

### BACKGROUND

X-linked chronic granulomatous disease (X-CGD) is an immunodeficiency caused by mutations in the CYBB gene on the X-chromosome, predominantly affecting males. CYBB encodes the gp91^{phox} (or NOX2) subunit of phagocyte NADPH oxidase, which is necessary for the production of reactive oxygen species (ROS) by neutrophils and other phagocytes for killing of select microbial organisms. Causative mutations can occur throughout the 13 exons or adjoining intronic splice sites of the >30-kb CYBB gene, resulting in defective or absent gp91^{phox} protein expression and loss of ROS production (1). X-CGD patients have recurring, life-threatening fungal and bacterial infections, hyper-inflammation, and granulomatous complications (2). Allogeneic transplantation of CD34⁺ hematopoietic stem/progenitor cells (HSPCs) can be curative for X-CGD, but graft-versus-host-disease remains a significant risk, and many patients lack a suitable matched donor. Autologous transplant of X-CGD patient HSPCs modified by random insertion of CYBB cDNA using retroviral vectors has demonstrated clinical benefit as salvage therapy for life-threatening infections, but has resulted in low levels of long-term gene marking in engrafted cells, as well as life-threatening myelodysplasia due to vector insertional mutagenesis. (3, 4). The use of constitutive promoters in these vectors for ectopic expression of gp91^{phox} carries an additional potential risk of aberrant production of ROS in corrected HSPCs, which might alter stem cell function or impair long-term hematopoietic engraftment (5, 6). In an effort to address these safety issues, a self-inactivating lentiviral vector has been developed (7) and is currently in clinical trials (8) for CYBB cDNA transfer using a chimeric myeloid-specific promoter for phagocyte-restricted expression; transduction of X-CGD patient HSPCs with this vector at ˜1 copy insertion per transduced cell has resulted in gp91^{phox} protein expression in HSPC-derived phagocytes at per-cell levels ˜50% of expression levels in healthy donor controls, with multiple vector copy inserts needed per cell to achieve physiologically normal expression levels (7).

Targeted gene insertion or gene editing using site-specific nucleases, including CRISPR (clustered regularly interspaced short palindromic repeats)/Cas9 (CRISPR-associated 9) nuclease (9-11), zinc-finger nucleases (ZFNs) (12-14), or transcription activator-like effector nucleases (TALENs) (15), has potential for gene therapy with a greatly reduced or absent risk of insertional mutagenesis. In these approaches, a DNA double-strand break (DSB) induced by the nuclease acts as a target for homology-directed repair (HDR) using a donor DNA template containing homologous sequences to those flanking the cut site. Due to the toxicity of double-stranded plasmid DNAs in transfections of primary cells (16-17), adeno-associated virus (AAV) vectors packaged with serotype 6 capsid (AAV6) are commonly used for transduction of HSPCs (18-24) to deliver long donor DNA templates (with an upper size limit of ˜4.7-kb for AAV vector packaging, including target site homologous sequences and ˜0.3-kb for the required AAV inverted terminal repeats or ITRs), while single-stranded oligodeoxynucleotides (ssODNs) have been used for gene repair or insertion of short donor templates (typically up to 100-200 nucleotides in length including homologous sequences) (24-29). The efficiency of targeted insertion of a donor DNA template is dependent upon the choice of DSB repair pathways between HDR and non-homologous end joining (NHEJ) (30), a more error-prone repair pathway that functions without a homologous donor template and can instead result in the formation of indels (insertion or deletion mutations) at the DSB site. This choice of repair pathway appears to be cell type (31) and cell cycle dependent, with HDR normally restricted to S and G2 phases of the cell cycle (32-33), which poses an additional challenge for HDR-mediated genome editing in quiescent hematopoietic stem cells.

Accordingly, key challenges remain for developing safe and effective medical therapies for X-CGD, including attaining clinically relevant targeted integration frequencies into LT-HSCs and attaining functional levels of protein expression and ROS production. There is therefore a need for new methods that allow for the successful treatment of X-CGD by overcoming such challenges. The present disclosure satisfies this need and provides other advantages as well.

### BRIEF SUMMARY

The present disclosure provides methods and compositions for treating X-linked Chronic Granulomatous Disease (X-CGD) in subjects, in particular through the genetic modification of cells taken from the subjects by integrating a functional copy of a CYBB cDNA at the endogenous CYBB locus in the cells, and subsequently reintroducing the modified cells back into the subject. In particular, the present methods and compositions involve the homologous-recombination-mediated introduction of functional, codon-optimized cDNAs into the genomes of cells at the CYBB locus, such that functional gp91^{phox} protein is expressed in the cells under the control of the endogenous CYBB promoter and other regulatory elements. In some embodiments, the CYBB cDNA is a full-length CYBB cDNA comprising exons 1-13 of the CYBB gene. In some embodiments, the CYBB cDNA is a partial CYBB cDNA, for example, a CYBB cDNA comprising exons 2-13 of the CYBB gene. In some embodiments, the regulatory elements controlling expression of the functional gp91^{phox} protein comprise endogenous intron 1 of the CYBB gene.

In one aspect, the present disclosure provides a method of genetically modifying a cell from a subject with X-linked chronic granulomatous disease (X-CGD), the method comprising: introducing into a cell isolated from the subject a single guide RNA (sgRNA) targeting the cytochrome b-245 beta chain (CYBB) gene, an RNA-guided nuclease, and a homologous donor template comprising a partial or full-length CYBB cDNA, flanked by a first and a second CYBB homology region; wherein: the sgRNA binds to the nuclease and directs it to a target sequence within the CYBB gene, whereupon the nuclease cleaves the gene at the target sequence, and wherein: the cDNA is integrated by homology directed recombination (HDR) at the site of the cleaved CYBB gene, such that the functional CYBB transcript is expressed under the control of the endogenous CYBB promoter, thereby providing functional gp91phox protein product in the cell. In some embodiments, the CYBB cDNA comprises a nucleotide sequence having at least 80% identity to SEQ ID NO:11.

In some embodiments, the method further comprises isolating the cell from the subject prior to the introducing of the sgRNA, RNA-guided nuclease, and homologous donor template. In some embodiments, the target sequence of the sgRNA is within exon 1 of the CYBB gene, and the CYBB cDNA comprises exons 1-13 of the CYBB gene. In some embodiments, the target sequence of the sgRNA is within exon 2 of the CYBB gene, and the CYBB cDNA comprises exons 2-13 of the CYBB gene. In some embodiments, the CYBB cDNA does not comprise exon 1 of the CYBB gene. In some embodiments, integration of the CYBB cDNA comprising exons 2-13 of the CYBB gene occurs downstream of endogenous intron 1 of the CYBB gene. In some embodiments, the sgRNA comprises a nucleotide sequence complementary to a sequence selected from the group consisting of SEQ ID NO:1, SEQ ID NO:2, SEQ ID NO:3, SEQ ID NO:4, SEQ ID NO:5, and SEQ ID NO:6. In some embodiments, the sgRNA comprises a nucleotide sequence complementary to SEQ ID NO: 3. In some embodiments, the sgRNA comprises a nucleotide sequence complementary to a sequence selected from the group consisting of SEQ ID NO:7, SEQ ID NO:8, SEQ ID NO:9, and SEQ ID NO:10. In some embodiments, the sgRNA comprises a nucleotide sequence complementary to SEQ ID NO:9.

In some embodiments, the sgRNA comprises 2′-O-methyl-3′-phosphorothioate (MS) modifications at one or more nucleotides. In some embodiments, the 2′-O-methyl-3′-phosphorothioate (MS) modifications are present at the three terminal nucleotides of the 5′ and 3′ ends. In some embodiments, the RNA-guided nuclease is a CRISPR-associated (Cas) nuclease, for example, Cas9. In some embodiments, the sgRNA and the RNA-guided nuclease are introduced into the cell as a ribonucleoprotein (RNP). In some embodiments, the RNP is introduced into the cell by electroporation. In some embodiments, the method further comprises introducing an inhibitor of non-homologous end joining (NHEJ) into the cell. In some embodiments, the NHEJ inhibitor is an inhibitor of 53BP1. In some embodiments, the inhibitor of 53BP1 is 53, or a variant thereof capable of inhibiting the activity of 53BP1. In some embodiments, the inhibitor of 53BP1, e.g. i53, is introduced by introducing an mRNA encoding the inhibitor. In some embodiments, the mRNA is introduced by electroporation together with the RNP.

In some embodiments, the CYBB cDNA comprises a nucleotide sequence having at least 85%, 90%, 95%, 96%, 97%, 98%, 99%, 99.5%, 99.7%, 99.9% or more identity to SEQ ID NO:11. In some embodiments, the CYBB cDNA comprises the nucleotide sequence of SEQ ID NO:11. In some embodiments, the homologous donor template further comprises a polyadenylation signal at the 3′ end of the cDNA, wherein both the cDNA and the polyadenylation signal are flanked by the first and the second CYBB homology regions on the template. In some embodiments, the polyadenylation signal is a bovine growth hormone polyadenylation signal or rabbit beta-globin polyadenylation signal. In some embodiments, the first and/or second CYBB homology region comprises nucleotides 1-400 or 2879-3279 of SEQ ID NO:12, or a contiguous portion of nucleotides 1-400 or 2879-3279 of SEQ ID NO:12. In some embodiments, the first and second CYBB homology regions comprise nucleotides 1-400 or 2879-3279 of SEQ ID NO:12. In some embodiments, the homologous template further comprises a woodchuck hepatitis virus post-transcriptional regulatory element (WPRE).

In some embodiments of the method, the homologous donor template comprises a nucleotide sequence having at least 80%, 85%, 90%, 95%, 96%, 97%, 98%, 99%, 99.5%, 99.7%, 99.9% or more identity to SEQ ID NO:12. In some embodiments, the homologous donor template is introduced into the cells using a recombinant adeno-associated virus (rAAV) serotype 6 vector. In some embodiments, the homologous donor template further comprises a selectable marker. In some embodiments, the cell is a CD34⁺ hematopoietic stem and progenitor cell (HSPC). In some embodiments, the CD34⁺ HSPC is isolated from the peripheral blood. In some embodiments, the expression of the integrated CYBB cDNA provides a level of functional gp91phox protein product in the cell that is at least 70%, 80%, 90%, or more of the level in a healthy control cell. In some embodiments, the expression of the integrated CYBB cDNA leads to a level of reactive oxygen species (ROS) production in the cell that is at least 10%, 20%, 30%, 40%, 50%, 60%, 70%, 80%, 90%, 100%, or more of the level in a healthy control cell as measured by the mean fluorescence intensity (MFI) of positive gated cells by dihydrorhodamine-123 (DHR) assay.

In another aspect, the present disclosure provides a method of treating a subject with X-CGD, comprising (i) genetically modifying a cell from the subject using any of the herein-described methods, and (ii) reintroducing the cell into the subject.

In some embodiments, the cell is reintroduced into the subject by systemic transplantation. In some embodiments, the systemic transplantation comprises intravenous administration. In some embodiments, the cell is reintroduced into the subject by local transplantation. In some embodiments, the local transplantation comprises intrafemoral or intrahepatic administration. In some embodiments, the cell is cultured and/or selected prior to being reintroduced into the subject.

In another aspect, the present disclosure provides an sgRNA that specifically targets exon 1 of the CYBB gene. In some embodiments, the sgRNA comprises a nucleotide sequence complementary to the sequence of any one of SEQ ID NOS:1-6.

In another aspect, the present disclosure provides an sgRNA that specifically targets exon 2 of the CYBB gene. In some embodiments, the sgRNA comprises a nucleotide sequence complementary to the sequence of any one of SEQ ID NOS:7-10.

In some embodiments of any of the herein-described sgRNAs, the sgRNA comprises 2′-O-methyl-3′-phosphorothioate (MS) modifications at one or more nucleotides. In some embodiments, the 2′-O-methyl-3′-phosphorothioate (MS) modifications are present at the three terminal nucleotides of the 5′ and 3′ ends.

In another aspect, the present disclosure provides a homologous donor template comprising: (i) a CYBB cDNA comprising a nucleotide sequence comprising at least 80%, 85%, 90%, 95%, 96%, 97%, 98%, 99%, 99.5%, 99.7%, 99.9% or more identity to SEQ ID NO:11; (ii) a first CYBB homology region located to one side of the cDNA within the donor template, and (iii) a second CYBB homology region located to the other side of the cDNA within the donor template.

In some embodiments, the first CYBB homology region comprises nucleotides 1-400 of SEQ ID NO:12, or a contiguous portion thereof, and the second CYBB homology region comprises nucleotides 2879-3279 of SEQ ID NO:12, or a contiguous portion thereof. In some embodiments, the CYBB cDNA comprises exons 1-13 of the CYBB gene. In some embodiments, the CYBB cDNA comprises exons 2-13 of the CYBB gene. In some embodiments, the CYBB cDNA comprises a nucleotide sequence having at least 85%, 90%, 95%, 96%, 97%, 98%, 99% or more identity to SEQ ID NO:11. In some embodiments, the CYBB cDNA is codon optimized. In some embodiments, the CYBB cDNA comprises the nucleotide sequence of SEQ ID NO:11.

In some embodiments, the donor template further comprises a polyadenylation signal at the 3′ end of the CYBB cDNA, wherein both the cDNA and the polyadenylation signal are flanked by the first and second CYBB homology regions on the template. In some embodiments, the polyadenylation signal is a bovine growth hormone polyadenylation signal or rabbit beta-globin polyadenylation signal. In some embodiments, the template comprises the sequence of SEQ ID NO: 12. In some embodiments, the donor template further comprises a selectable marker. In some embodiments, the donor template further comprises a woodchuck hepatitis virus post-transcriptional regulatory element (WPRE).

In another aspect, the present disclosure provides an isolated HSPC comprising any of the herein-described sgRNAs and/or homologous donor templates.

In another aspect, the present disclosure provides an isolated, genetically modified HSPC comprising an exogenous, codon-optimized CYBB cDNA integrated at the endogenous CYBB gene. In some embodiments, the integrated cDNA comprises a nucleotide sequence having at least 80% identity to SEQ ID NO:11.

In some embodiments, the CYBB cDNA comprises a nucleotide sequence having at least 85%, 90%, 95%, 96%, 97%, 98%, 99%, 99.5%, 99.7%, 99.9% or more identity to SEQ ID NO:11. In some embodiments, the CYBB cDNA comprises the nucleotide sequence of SEQ ID NO:11. In some embodiments, the exogenous CYBB cDNA comprises exons 1-13, and wherein the cDNA is integrated within exon 1 of the endogenous CYBB gene. In some embodiments, the exogenous CYBB cDNA comprises exons 2-13, and the cDNA is integrated within exon 2 of the endogenous CYBB gene. In some embodiments, the HSPC was modified using any of the herein-described methods.

### BRIEF DESCRIPTION OF THE DRAWINGS

FIGS. 1A-1C. Schemata of three correction strategies for targeted CYBB cDNA insertion in X-CGD patient CD34⁺ HSPCs. (FIG. 1A) Strategy 1 utilizes CRISPR/Cas9 CYBB1-g3 sgRNA and an ˜2.7-kb AAV6 donor template containing CYBB exon 1-13 cDNA and bGH poly-A (pA) for targeted insertion at CYBB exon 1 (CYBB1-13-pA). (FIG. 1B) Strategy 2 utilizes CRISPR/Cas9 CYBB2-g8 sgRNA and an ˜3.3-kb AAV6 donor template containing CYBB exon 2-13 cDNA with WPRE and bGH pA for targeted insertion at exon 2 (CYBB2-13-WPRE-pA). (FIG. 1C) Strategy 3 utilizes CRISPR/Cas9 CYBB2-g3 sgRNA and an ˜3-kb AAV6 donor template containing CYBB exon 2-13 cDNA with rabbit beta-globin (b-glob) pA for insertion at exon 2 (CYBB2-13-pA). Each donor template includes 5′ and 3′ homology arms (HA) of ˜400-nucleotides each, flanking the cDNA expression cassette; the listed donor templates sizes do not include the sizes of the AAV ITRs required for viral packaging. Depicted for each strategy are the naive CYBB locus (top) including the CRISPR/Cas9 target site (with the PAM sequence underlined), the AAV6 donor construct (middle), and the CYBB locus after targeted insertion (bottom); for simplicity, only the first 6 exons of the CYBB locus are depicted for strategies 2 and 3.

FIGS. 2A-2C. ICE analysis of indel patterns for sgRNAs from three CYBB targeting strategies. Detailed information including the indel size (positive for insertions, negative for deletions: 0 for the unaltered endogenous sequence) and normalized relative contribution of each indel sequence are shown from representative replicates of CRISPR/Cas9 targeting studies performed in male HSPCs in the absence of AAV6 donor templates using (FIG. 2A) CYBB1-g3 sgRNA for correction strategy 1, (FIG. 2B) CYBB2-g8 sgRNA for correction strategy 2, or (FIG. 2C) CYBB2-g3 sgRNA for correction strategy 3. The 5′ to 3′ sequences shown match the orientation of the CYBB coding strand, and correspond to the reverse complement of the sgRNA sequences for strategies 1 and 3.

FIGS. 3A-3F. Initial comparisons of functional correction using three strategies for targeted insertion in X-CGD patient HSPCs without optimization of targeted insertion conditions. (FIG. 3A) Percent indel frequency by CRISPR/Cas9 using CYBB1-g3 (strategy 1), CYBB2-g8 (strategy 2), and CYBB2-g3 (strategy 3) sgRNAs in male X-CGD patient or healthy donor HSPCs. (FIG. 3B) Targeted insertion frequency analysis by ddPCR in HSPCs at 48 hours post-electroporation (nucleofection) of CRISPR/Cas9 and transduction with AAV6 donor template. (FIG. 3C) In vitro differentiation of HSPCs into phagocytes, resulting in a mixture of polymorphonuclear neutrophils (‘n’) and macrophages (‘m’), as visualized by Giemsa staining. (FIGS. 3D-3F) Flow cytometry immunostaining analysis of gp91^{phox} expression in differentiated phagocytes after correction of X-CGD patient HSPCs by targeted insertion of: (FIG. 3D) CYBB exon 1-13 cDNA for correction strategy 1, (FIG. 3E) exon 2-13 cDNA for correction strategy 2, or (FIG. 3F) exon 2-13 cDNA for correction strategy 3. Data shown are from representative replicates. Y-axes represent side-scatter (SSC) measurements. Healthy donor controls and uncorrected X-CGD (naive) patient controls are also shown.

FIGS. 4A-4F. Optimization of targeted insertion efficiency using i53 mRNA in HSPCs. All data shown are from targeted insertion using correction strategy 3. (FIG. 4A) Percent indel frequency by ICE analysis in healthy donor HSPCs at 3-5 days after electroporation of Cas9 RNP with or without 10 μg (48 pmol) of i53 mRNA (bars denote mean±SD; n=5; ns=not significant by two-tailed paired t-test). (FIG. 4B) Targeted insertion frequency analysis by ddPCR in healthy donor HSPCs at 3-5 days after electroporation of Cas9 RNP (with or without 10 μg of i53 mRNA) and transduction with AAV6 donor template at an MOI of 2×10⁴, 5×10⁴, or 1×10⁵ (bars denote mean±SD, for mean targeted insertion frequencies of 13.7% without i53 versus 21.7% with i53 at 2×10⁴ MOI of AAV, 18.1% without i53 versus 25.5% with i53 at 5×10⁴ MOI, and 31.0% without i53 versus 46.7% with i53 at 1×10⁵ MOI; n=4 for 2×10⁴ and 5×10⁴ MOI groups; n=6 for “no AAV” and 1×10⁵ MOI groups; *p<0.05 or **p<0.01 by two-tailed paired t-test). (FIG. 4C) Cell viability by trypan blue exclusion stain at 3 days after electroporation of Cas9 RNP with or without 10 μg of i53 mRNA, and with or without AAV6 transduction at 1×10⁵ MOI (bars denote mean±SD; n=3; ns=not significant by two-tailed unpaired t-test. (FIG. 4D) Cell viability by trypan blue exclusion stain at 3 days after electroporation and AAV6 transduction at the indicated MOIs (groups include pooled data from electroporations with or without i53 mRNA; bars denote mean±SD; ****p<0.0001 by one-way ANOVA). (FIG. 4E) Representative replicates from side-by-side comparisons of the effect of i53 mRNA co-transfection on correction of X-CGD patient HSPCs using AAV6 transduction at 1×10⁵ MOI, analyzed by gp91^{phox} expression following phagocyte differentiation. (FIG. 4F) Summary of the enhancement by i53 mRNA co-transfection on correction of X-CGD patient HSPCs using AAV6 transduction at 1×10⁵ MOI, analyzed by gp91^{phox} expression following phagocyte differentiation (bars denote mean±SD, for mean correction efficiencies of 20.2% gp91^{phox+} cells without i53 versus 30.3% with i53; n=4; *p<0.05 by two-tailed unpaired t-test).

FIGS. 5A-5E. Comparison of correction strategy 1+WPRE with strategies 2 and 3 using optimized targeted insertion conditions. (FIG. 5A) ddPCR analysis of targeted insertion frequency in X-CGD patient or healthy donor HSPCs (bars denote mean±SD, with mean insertion efficiencies of 45.8% for strategy 1+WPRE, 50.9% for strategy 2, and 46.7% for strategy 3; n=8 for naive HSPCs; n=3 for strategy 1+WPRE; n=6 for strategies 2 and 3). (FIG. 5B) Correction of X-CGD patient HSPCs demonstrated by functional restoration of gp91^{phox} expression in differentiated phagocytes by flow cytometry analysis (bars denote mean±SD, with mean correction efficiencies of 6.5% gp91^{phox+} cells for strategy 1+WPRE, 47.6% for strategy 2, and 33.3% for strategy 3, versus 0.5% for X-CGD naive; n=3 for strategy 1+WPRE; n=5 for X-CGD naive, strategy 2, and strategy 3; *p<0.05 or ****p<0.0001 by two-tailed unpaired t-test). (FIG. 5C) Representative replicate flow cytometry immunostaining analysis of gp91^{phox} expression in phagocytes differentiated from X-CGD patient HSPCs corrected by strategy 1+WPRE; also shown are healthy donor and X-CGD naive controls, as well as MFIs of the gated gp91^{phox+} cell populations for the corrected and healthy control phagocytes. (FIG. 5D) Representative replicate flow cytometry analyses of per-cell levels of gp91^{phox} expression in phagocytes differentiated from corrected X-CGD patient HSPCs by strategy 2 (left) or strategy 3 (right); black=uncorrected X-CGD naive control; gray=corrected; white=healthy donor control; also shown are MFIs of the gated gp91^{phox+} cell populations for the corrected and healthy control phagocytes. (FIG. 5E) Summary of relative gp91^{phox} expression data in corrected cells for strategy 1+WPRE, strategy 2, and strategy 3, computed from flow cytometry data as the MFI of corrected cells relative to healthy controls for the gated gp91^{phox+} phagocyte populations (bars denote mean±SD; n=3 for strategy 1+WPRE; n=5 for strategy 2; n=7 for strategy 3; *p<0.05 or ***p<0.001 by two-tailed unpaired t-test).

FIGS. 6A-6B. ROS production as a measure of oxidative function in phagocytes differentiated from corrected X-CGD patient HSPCs. Comparison of per-cell levels of ROS production by DHR flow cytometry assay in functionally corrected phagocytes for (FIG. 6A) strategy 1+WPRE and (FIG. 6B) strategy 2 and strategy 3; also shown are MFIs of the gated DHR⁺ cell populations for the corrected and healthy control phagocytes.

FIGS. 7A-7F. Engraftment data of X-CGD patient HSPCs corrected using strategy 2 under optimized targeted insertion conditions and transplanted into NSG mice. (FIG. 7A) In vitro data of the correction efficiency in the X-CGD patient HSPC population used for transplant, measured by gp91^{phox} expression in phagocytes following in vitro differentiation of untransplanted cells. (FIG. 7B) In vivo engraftment data from the peripheral blood of a representative mouse at 12 weeks post-transplant of corrected X-CGD HSPCs, immunostained for human CD45 (hCD45; left) as a marker of human hematopoietic cell engraftment and human gp91^{phox} (right; shown are gp91^{phox+} cells gated from the human CD45⁺ cell population) as a measure of corrected human phagocytes arising from engrafted HSPCs. (FIG. 7C) Summary of human CD45⁺ cell engraftment and (FIG. 7D) human gp91^{phox+} cells gated from the human CD45⁺ population in peripheral blood of NSG mice at 12 weeks post-transplant (bars denote mean±SD; n=4 for healthy control and X-CGD naive HSPC transplants; n=6 for corrected X-CGD HSPC transplants; ****p<0.0001 by two-tailed unpaired t-test). (FIG. 7E) Human CD34⁺ cells harvested and sorted from mouse bone marrow at 16 weeks post-transplant and differentiated in vitro to generate mature phagocytes for DHR assay of ROS production as a measure of functional correction in engrafted CD34⁺ HSPCs (bars denote mean±SD; n=4 for healthy control and X-CGD naive; n=6 for corrected X-CGD; *p<0.05 by two-tailed unpaired t-test). (FIG. 7F) Summary of mean per-cell levels of ROS production in gated DHR⁺ corrected neutrophils from (FIG. 7E), calculated relative to healthy control neutrophils based on MFIs (bars denote mean±SD; n=6).

FIGS. 8A-8B. Screening and Characterization of gp91 sgRNA guides for exon 1.

FIG. 8A: Percentage INDELS at day 4 post-nucleofection. FIG. 8B: Target sequences of Exon 1 sgRNAs.

FIGS. 9A-9C. Strategies for Developing a Clinical Donor for gp91 gene, strategy 1 (Exon 1-Exon 13). FIG. 9A: Structure of CYBB gene, and FIG. 9B: Donor templates comprising Exons 1-13, along with Intron I (templates III and IV) and WPRE (templates II and IV). FIG. 9C: Percentage of healthy CD34+ HSPCs showing homologous recombination at day 4 using templates I-IV).

FIGS. 10A-10C. Gp91 expression at 14 days post myeloid differentiation, with strategy 1 (Exons 1-13) and donor templates I-IV. Gp91-expressing cells as detected by FACS without templates (FIG. 10A) or with templates I-IV (FIG. 10B). Intron 1 of the beta globin gene (HBB) was cloned between exon 1 and exon 2 in place of the gp91 intron 1 (III and IV). This approach tests whether regulatory sequences present in the HBB intron 1 could replace those of gp91 intron 1 in support of gp91 expression. In addition, the presence of WPRE (woodchuck hepatitis virus), a posttranslational regulatory sequence, was added upstream of the exogenous polyA sequence in order to enhance protein expression (II and IV). FIG. 10C shows the percent GT driving gp91 expression.

FIG. 11. Percentage of cells showing gp91 expression, with control cells (healthy donor, X-CGH patient-derived donors), and with templates I or II.

FIGS. 12A-12D. Strategies for Developing a Clinical Donor for gp91 gene, strategy 2 (Exon 2-Exon 13). FIG. 12A. Strategy 2 template. This strategy offers the following advantages: i) it uses a partial (exons 2-13) corrective cDNA introduced at the start site of exon 2; ii) it retains the 5′ UTR and intron 1 regulatory sequences; and iii) protein expression should reach that of WT CYBB levels. This strategy does not correct, however, pathogenic mutations in the 5′UTR or in Exon 1 of CYBB (which are less prevalent in any case). FIG. 12B: Target sequences of Exon 2 sgRNAs. FIG. 12C: Percentage of INDELS and percent viability at day 4 using the different Exon 2 sgRNAs. FIG. 12D. Percentage of homologous recombination at day 4 using fresh (healthy donors) and frozen male X-CGD-patient-derived CD34⁺ HSPCs.

FIG. 13. Map of exemplary AAV6 vector comprising a homologous donor template for correcting exons 2-13 of X-CGD (comprising, e.g., codon optimized cDNA sequence shown as SEQ ID NO: 11).

FIG. 14. Gp91 expression at 14 days post-myeloid differentiation in control cells and in cells with an exon 2-13 donor, from healthy donors genome targeted with Exon 2-13 template.

FIGS. 15A-15B. FIG. 15A: DHR assay to detect ROS production. FIG. 15B: ROS activity in healthy control cells, in cells from CGD patients, and in corrected CGD cells with introduced Exon 2-13+WPRE template.

FIGS. 16A-16B. Gp91 protein expression and DHR assay at 14 days post myeloid differentiation, using cells from X-CGD patient #1 (FIG. 16A) and patient #2 (FIG. 16B).

### DETAILED DESCRIPTION

### 1. Introduction

The present disclosure provides methods and compositions for the treatment of X-linked Chronic Granulomatous Disease (X-CGD) Deficiency in subjects, through the introduction and integration at the endogenous Cytochrome b 245 gene (gp91, CYBB) of functional, codon-optimized partial or full-length CYBB cDNAs. The methods involve the introduction of ribonucleoproteins (RNPs) comprising single guide RNAs (sgRNAs) and RNA-guided nucleases (e.g., Cas9) into cells from the subject, as well as the introduction of homologous templates for repair. The cDNAs are integrated at the endogenous CYBB gene, e.g., at the start site, or within the second exon, such that the cDNA is expressed under the control of the endogenous CYBB promoter and other regulatory elements and functional gp91^{phox} protein is produced in the cell, thereby compensating for a genetic deficiency in the subject. In particular embodiments, the regulatory elements controlling expression of functional gp91^{phox} protein includes endogenous intron 1 of the CYBB gene.

In particular embodiments, the RNP complexes, e.g., complexes comprising CYBB-targeting sgRNA and Cas9 protein, are delivered to cells via electroporation, followed by the transduction of the homologous template using an AAV6 viral vector. In some embodiments, an inhibitor of non-homologous end joining, for example, an mRNA encoding i53 is also transduced by electroporation, e.g., at the same time as the RNP. The homologous templates for repair are constructed to have arms of homology centered around the cut site within the CYBB locus, located on either side of the cDNA on the template. Transcription is terminated using an exogenous polyadenylation signal. This system can be used to modify any human cell, and in particular embodiments CD34⁺ HSPCs are used.

### 2. General

Practicing the present methods utilizes routine techniques in the field of molecular biology. Basic texts disclosing the general methods of use in the present methods and compositions include Sambrook and Russell, Molecular Cloning, A Laboratory Manual (3^{rd} ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid”. “nucleotide” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase 11 type promoter, a TATA element. A promoter also optionally includes distal enhancer or repressor elements, which can be located as much as several thousand base pairs from the start site of transcription. The promoter can be a heterologous promoter.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a polynucleotide or polypeptide is “heterologous” to an organism if the polynucleotide or polypeptide originates from a foreign species compared to the organism, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “expression” and “expressed” refer to the production of a transcriptional and/or translational product, e.g., of an CYBB cDNA or encoded protein. In some embodiments, the term refers to the production of a transcriptional and/or translational product encoded by a gene or a portion thereof. The level of expression of a DNA molecule in a cell may be assessed on the basis of either the amount of corresponding mRNA that is present within the cell or the amount of protein encoded by that DNA produced by the cell.

“CYBB” or “Cytochrome b-245” or “gp9l” refers to a gene encoding the “gp91^{phox}” or “cytochrome b-245” protein, which is a subunit of NADPH oxidase. CYBB is located on the short arm of the X chromosome, at Xp21.1-p11.4. It is mutated in patients, primarily males, with X-linked Chronic Granulomatous Disease (CGD), e.g., missense mutations, nonsense mutations, insertions, deletions, and splicing mutations, resulting in a lack of gene expression or the expression of nonfunctional protein. The CYBB cDNAs used in the present methods are typically either full-length (i.e., comprising exons 1-13) or spanning exons 2 to 13. Integration of the cDNA using the present methods allow cells of the patient to express functional gp91^{phox} protein and thus restore protein activity in patients. The accession number for the human CYBB gene is NCBI Gene ID 1536, and for the encoded protein it is UniProt P04839. A codon-optimized (or “codon diverged”) version of the CYBB cDNA, comprising 78% sequence homology to the endogenous, wild-type gene, is shown as SEQ ID NO:11. The present methods can be used with any patient with X-CGD, with any CYBB mutation, so long that the CYBB locus retains a functional promoter and potentially other regulatory elements such that the integrated cDNA is expressed in cells from the patient.

As used herein, “functional CYBB cDNA” refers to a partial or full-length cDNA, the integration of which into a CYBB locus using the methods described herein enables production of functional “gp91^{phox}” or “cytochrome b-245” protein from that locus. As used herein, “functional gp91^{phox}” or “functional cytochrome b-245” protein refers to a protein having similar or equivalent protein function as the wild-type gp91^{phox}/cytochrome b-245 protein (UniProt P4839). In some embodiments, functional gp91^{phox} protein has at least 50%, 60%, 70%, 80%, 90% or 100% of the function of wild-type gp91^{phox} protein, as determined by any method known in the art for assessing gp91^{phox} protein function, including but not limited to assessment of cellular ROS production, which is described in the Examples below.

“X-CGD,” or “X-linked chronic granulatous disease.” is an X-linked immunodeficiency disorder caused by mutations in the CYBB gene. The product of the CYBB gene, i.e., the cytochrome b beta chain, or gp91^{phox}, is a subunit of NADPH oxidase, and CYBB mutations in X-CGD patients result in little or no protein function and/or production. The loss of NADPH activity prevents neutrophils and other phagocytes from being able to produce reactive oxygen species (ROS), leaving the patient vulnerable to infection. Any of a variety of mutations in CYBB, including missense mutations, nonsense mutations, insertions, deletions, and splicing mutations, can prevent the expression of functional encoded protein. The present methods can compensate for the deficiencies caused by such CYBB mutations in patients, regardless of the nature or location of the mutations.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants of a protein can have an increased stability, assembly, or activity as described herein.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C). Methionine (M)
   (see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the N-terminal residue, which is numbered 1, in an unmodified wild-type polypeptide sequence.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93%, 94%, 95%, 96%, 97%, 98%, 99%, 99.5%, 99.7%, 99.9% or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215: 403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acids. CRISPR-Cas systems are found in a wide range of bacterial and archaeal organisms. CRISPR-Cas systems fall into two classes with six types, I, II, III, IV, V. and VI as well as many sub-types, with Class 1 including types I and III CRISPR systems, and Class 2 including types II, IV, V and VI; Class 1 subtypes include subtypes I-A to I-F, for example. See, e.g., Fonfara et al., Nature 532, 7600 (2016), Zetsche et al., Cell 163, 759-771 (2015); Adli et al. (2018). Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are effected by multiple Cas proteins, with Cas3 providing the endonuclease activity, whereas in class 2 systems they are all carried out by a single Cas, Cas9.

A “homologous repair template” refers to a polynucleotide sequence that can be used to repair a double stranded break (DSB) in the DNA, e.g., a CRISPR/Cas9-mediated break at the CYBB locus as induced using the herein-described methods and compositions. The homologous repair template comprises homology to the genomic sequence surrounding the DSB, i.e., comprising CYBB homology arms as described herein. In some embodiments, two distinct homologous regions are present on the template, with each region comprising at least 50, 100, 200, 300, 400, 500, 600, 700, 800, 900 or more nucleotides or more of homology with the corresponding genomic sequence. In particular embodiments, the templates comprise two homology arms comprising about 500 nucleotides of homology extending from either site of the sgRNA target site. The repair template can be present in any form, e.g., on a plasmid that is introduced into the cell, as a free floating doubled stranded DNA template (e.g., a template that is liberated from a plasmid in the cell), or as single stranded DNA. In particular embodiments, the template is present within a viral vector, e.g., an adeno-associated viral vector such as AAV6. The templates of the present disclosure also comprise a full-length, codon-optimized CYBB cDNA, as well as, typically, a polyadenylation signal such as from bovine growth hormone or rabbit beta-globin.

As used herein, “homologous recombination” or “HR” refers to insertion of a nucleotide sequence during repair of double-strand breaks in DNA via homology-directed repair mechanisms. This process uses a “donor template” or “homologous repair template” with homology to nucleotide sequence in the region of the break as a template for repairing a double-strand break. The presence of a double-stranded break facilitates integration of the donor sequence. The donor sequence may be physically integrated or used as a template for repair of the break via homologous recombination, resulting in the introduction of all or part of the nucleotide sequence. This process is used by a number of different gene editing platforms that create the double-strand break, such as meganucleases, such as zinc finger nucleases (ZFNs), transcription activator-like effector nucleases (TALENs), and the CRISPR-Cas9 gene editing systems. In particular embodiments. HR involves double-stranded breaks induced by CRISPR-Cas9. In further embodiments, the CRISPR-Cas9 comprises high-fidelity Cas9 variants having improved on-target specificity and reduced off-target activity. Examples of high-fidelity Cas9 variants include but are not limited to those described in PCT Publication Nos. WO/2018/068053 and WO/2019/074542, each of which is herein incorporated by reference in its entirety.

### 4. CRISPR/Cas Systems Targeting the CYBB Locus

The present disclosure provides methods and compositions for integrating functional CYBB cDNAs into the endogenous CYBB locus in cells from a subject with X-CGD. In particular embodiments, the cells are hematopoietic stem and progenitor cells (HSPCs). The cells can be modified using the methods described herein and then reintroduced into the subject, wherein the expression of the cDNA in the modified cells in vivo can restore protein function and activity and, consequently, ROS production that is missing or deficient in the subject with X-CGD.

The present disclosure is based in part on the identification of CRISPR guide sequences that specifically and effectively direct the cleavage of CYBB, e.g., within exon 1 or exon 2 of CYBB, by RNA-guided nucleases such as Cas9. In particular embodiments, the methods involve the introduction of ribonucleoproteins (RNPs) comprising an sgRNA targeting CYBB and Cas9, as well as a template DNA molecule comprising CYBB homology arms flanking a functional, codon-optimized CYBB cDNA comprising exons 1-13 or exons 2-13. Using the present methods, high rates of targeted integration at the CYBB locus (e.g., 20%, 30%, or more) and expression of the cDNA (e.g., producing 70%, 75%, 80%, 85%, 90%, 95%, 100% or more of the level of gp91^{phox} and/or ROS production of the level in control cells from a healthy individual) can be achieved, with the result that the transplantation and long-term engraftment of the modified cells can lead to a reduction or elimination of symptoms caused by the protein deficiency associated with X-CGD.

sgRNAs

The single guide RNAs (sgRNAs) used in the present methods and compositions target the CYBB locus. sgRNAs interact with a site-directed nuclease such as Cas9 and specifically bind to or hybridize to a target nucleic acid within the genome of a cell, such that the sgRNA and the site-directed nuclease co-localize to the target nucleic acid in the genome of the cell. The sgRNAs as used herein comprise a targeting sequence comprising homology (or complementarity) to a target DNA sequence at the CYBB locus, and a constant region that mediates binding to Cas9 or another RNA-guided nuclease. The sgRNA can target any sequence within CYBB adjacent to a PAM sequence. In some embodiments, the target sequence is within exon 1 of CYBB, e.g., when a donor template comprising exons 1-13 is used. In some such embodiments, the target sequence of the sgRNA targets one of the sequences shown as SEQ ID NOS:1-6, or a sequence having, e.g., at least 90%, 91%, 92%, 93%, 94%, 95%, 96%, 97%, 98%, 99%, or more identity to, e.g., comprising 1, 2, 3, or more nucleotide substitutions, additions, or subtractions relative to, one of SEQ ID NOS:1-6. In particular embodiments, the sgRNA targets the sequence shown as SEQ ID NO:3. In some embodiments, the target sequence is within exon 2 of CYBB, e.g., when a donor template comprising exons 2-13 is used. In such embodiments, regulatory sequences within the 5′UTR and intron 1 are retained and can help regulate the expression of the integrated cDNA. In some such embodiments, the target sequence of the sgRNA comprises one of the sequences shown as SEQ ID NOS: 7-10, or a sequence having, e.g., at least 90%, 91%, 92%, 93%, 94%, 95%, 96%, 97%, 98%, 99%, or more identity to, e.g., comprising 1, 2, 3, or more nucleotide substitutions, additions, or subtractions relative to, one of SEQ ID NOS:7-10. In particular embodiments, the target sequence is shown as SEQ ID NO: 9.

The targeting sequence of the sgRNAs may be, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or 15-25, 18-22, or 19-21 nucleotides in length, and shares homology with a targeted genomic sequence, in particular at a position adjacent to a CRISPR PAM sequence. The sgRNA targeting sequence is designed to be homologous to the target DNA, i.e., to share the same sequence with the non-bound strand of the DNA template or to be complementary to the strand of the template DNA that is bound by the sgRNA. The homology or complementarity of the targeting sequence can be perfect (i.e., sharing 100% homology or 100% complementarity to the target DNA sequence) or the targeting sequence can be substantially homologous (i.e., having less than 100% homology or complementarity, e.g., with 1-4 mismatches with the target DNA sequence).

Each sgRNA also includes a constant region that interacts with or binds to the site-directed nuclease, e.g., Cas9. In the nucleic acid constructs provided herein, the constant region of an sgRNA can be from about 70 to 250 nucleotides in length, or about 75-100 nucleotides in length, 75-85 nucleotides in length, or about 80-90 nucleotides in length, or 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length. The overall length of the sgRNA can be, e.g., from about 80-300 nucleotides in length, or about 80-150 nucleotides in length, or about 80-120 nucleotides in length, or about 90-110 nucleotides in length, or, e.g, 90, 91, 92, 93, 94, 95, 96, 97, 98, 99, 100, 101, 102, 103, 104, 105, 106, 107, 108, 109, or 110 nucleotides in length.

It will be appreciated that it is also possible to use two-piece gRNAs (cr:tracrRNAs) in the present methods, i.e., with separate crRNA and tracrRNA molecules in which the target sequence is defined by the crispr RNA (crRNA), and the tracrRNA provides a binding scaffold for the Cas nuclease.

In some embodiments, the sgRNAs comprise one or more modified nucleotides. For example, the polynucleotide sequences of the sgRNAs may also comprise RNA analogs, derivatives, or combinations thereof. For example, the probes can be modified at the base moiety, at the sugar moiety, or at the phosphate backbone (e.g., phosphorothioates). In some embodiments, the sgRNAs comprise 3′ phosphorothiate internucleotide linkages, 2′-O-methyl-3′-phosphoacetate modifications, 2′-fluoro-pyrimidines, S-constrained ethyl sugar modifications, or others, at one or more nucleotides. In particular embodiments, the sgRNAs comprise 2′-O-methyl-3′-phosphorothioate (MS) modifications at one or more nucleotides (see, e.g., Hendel et al. (2015) Nat. Biotech. 33(9):985-989, the entire disclosure of which is herein incorporated by reference). In particular embodiments, the 2′-O-methyl-3′-phosphorothioate (MS) modifications are at the three terminal nucleotides of the 5′ and 3′ ends of the sgRNA.

The sgRNAs can be obtained in any of a number of ways. For sgRNAs, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

### RNA-Guided Nucleases

Any CRISPR-Cas nuclease can be used in the method, i.e., a CRISPR-Cas nuclease capable of interacting with a guide RNA and cleaving the DNA at the target site as defined by the guide RNA. In some embodiments, the nuclease is Cas9 or Cpf1. In particular embodiments, the nuclease is Cas9. The Cas9 or other nuclease used in the present methods can be from any source, so long that it is capable of binding to an sgRNA of the present disclosure and being guided to and cleaving the specific CYBB sequence targeted by the targeting sequence of the sgRNA. In particular embodiments, the Cas9 is from Streptococcus pyogenes.

Also disclosed herein are CRISPR/Cas or CRISPR/Cpf1 systems that target and cleave DNA at the CYBB locus. An exemplary CRISPR/Cas system comprises (a) a Cas (e.g., Cas9) or Cpf1 polypeptide or a nucleic acid encoding said polypeptide, and (b) an sgRNA that hybridizes specifically to CYBB, or a nucleic acid encoding said guide RNA. In some instances, the nuclease systems described herein, further comprises a donor template as described herein. In particular embodiments, the CRISPR/Cas system comprises an RNP comprising an sgRNA targeting CYBB and a Cas protein such as Cas9.

In addition to the CRISPR/Cas9 platform (which is a type 11 CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9), Streptococcus thermophilus Cas9 (StCas9), Campylobacter jejuni Cas9 (CjCas9) and Neisseria cinerea Cas9 (NcCas9) to name a few. Alternatives to the Cas system include the Francisella novicida Cpf1 (FnCpf1), Acidaminococcus sp. Cpf1 (AsCpf1), and Lachnospiraceae bacterium ND2006 Cpf1 (LbCpf1) systems. Any of the above CRISPR systems may be used to induce a single or double stranded break at the CYBB locus to carry out the methods disclosed herein.

Introducing the sgRNA and Cas Protein into Cells

The sgRNA and nuclease can be introduced into a cell using any suitable method, e.g., by introducing one or more polynucleotides encoding the sgRNA and the nuclease into the cell, e.g., using a vector such as a viral vector or delivered as naked DNA or RNA, such that the sgRNA and nuclease are expressed in the cell. In some embodiments, one or more polynucleotides encoding the sgRNA, the nuclease or a combination thereof are included in an expression cassette. In some embodiments, the sgRNA, the nuclease, or both sgRNA and nuclease are expressed in the cell from an expression cassette. In some embodiments, the sgRNA, the nuclease, or both sgRNA and nuclease are expressed in the cell under the control of a heterologous promoter. In some embodiments, one or more polynucleotides encoding the sgRNA and the nuclease are operatively linked to a heterologous promoter. In particular embodiments, the sgRNA and nuclease are assembled into ribonucleoproteins (RNPs) prior to delivery to the cells, and the RNPs are introduced into the cell by, e.g., electroporation.

Animal cells, mammalian cells, preferably human cells, modified ex vivo, in vitro, or in vivo are contemplated. Also included are cells of other primates; mammals, including commercially relevant mammals, such as cattle, pigs, horses, sheep, cats, dogs, mice, rats; birds, including commercially relevant birds such as poultry, chickens, ducks, geese, and/or turkeys.

In some embodiments, the cell is an embryonic stem cell, a stem cell, a progenitor cell, a pluripotent stem cell, an induced pluripotent stem (iPS) cell, a somatic stem cell, a differentiated cell, a mesenchymal stem cell or a mesenchymal stromal cell, a neural stem cell, a hematopoietic stem cell or a hematopoietic progenitor cell, an adipose stem cell, a keratinocyte, a skeletal stem cell, a muscle stem cell, a fibroblast, an NK cell, a B-cell, a T cell, or a peripheral blood mononuclear cell (PBMC). In particular embodiments, the cells are CD34⁺ hematopoietic stem and progenitor cells (HSPCs), e.g., cord blood-derived (CB), adult peripheral blood-derived (PB), or bone marrow derived HSPCs.

HSPCs can be isolated from a subject, e.g., by collecting mobilized peripheral blood and then enriching the HSPCs using the CD34 marker. In some embodiments, the cells are from a subject with X-CGD. In some embodiments, a method is provided of treating a subject with X-CGD, comprising genetically modifying a plurality of HSPCs isolated from the subject so as to integrate a partial or full-length CYBB cDNA at the CYBB locus, and reintroducing the HSPCs into the subject.

To avoid immune rejection of the modified cells when administered to a subject, the cells to be modified are preferably derived from the subject's own cells. Thus, preferably the mammalian cells are autologous cells from the subject to be treated with the modified cells. In some embodiments, however, the cells are allogeneic, i.e., isolated from an HLA-matched or HLA-compatible, or otherwise suitable, donor.

In some embodiments, cells are harvested from the subject and modified according to the methods disclosed herein, which can include selecting certain cell types, optionally expanding the cells and optionally culturing the cells, and which can additionally include selecting cells that contain the transgene integrated into the CYBB locus. In particular embodiments, such modified cells are then reintroduced into the subject.

Further disclosed herein are methods of using said nuclease systems to produce the modified host cells described herein, comprising introducing into the cell (a) an RNP as described herein that targets and cleaves DNA at the CYBB locus, and (b) a homologous donor template or vector as described herein. Each component can be introduced into the cell directly or can be expressed in the cell by introducing a nucleic acid encoding the components of said one or more nuclease systems.

Such methods will target integration of the functional CYBB cDNA at the endogenous CYBB locus in a host cell ex vivo. Such methods can further comprise (a) introducing a donor template or vector into the cell, optionally after expanding said cells, or optionally before expanding said cells, and (b) optionally culturing the cell.

In some embodiments, the disclosure herein contemplates a method of producing a modified mammalian host cell, the method comprising introducing into a mammalian cell: (a) an RNP comprising a Cas nuclease such as Cas9 and an sgRNA specific to the CYBB locus, and (b) a homologous donor template or vector as described herein. The disclosure further contemplates a mammalian host cell composition, wherein the mammalian host cell comprises: (a) an RNP comprising a Cas nuclease such as Cas9 and an sgRNA specific to the CYBB locus, and (b) a homologous donor template or vector as described herein.

In any of these methods, the nuclease can produce one or more single stranded breaks within the CYBB locus, or a double stranded break within the CYBB locus. In these methods, the CYBB locus is modified by homologous recombination with said donor template or vector to result in insertion of the transgene into the locus. The methods can further comprise (c) selecting cells that contain the transgene integrated into the CYBB locus.

In some embodiments, i53 (Canny et al. (2018) Nat Biotechnol 36:95) is introduced into the cell in order to promote integration of the donor template by homology directed repair (HDR) versus integration by non-homologous end-joining (NHEJ). For example, an mRNA encoding i53 can be introduced into the cell, e.g., by electroporation at the same time as an sgRNA-Cas9 RNP. The sequence of i53 can be found, inter alia, at www.addgene.org/92170/sequences/.

Techniques for insertion of transgenes, including large transgenes, capable of expressing functional proteins, including enzymes, cytokines, antibodies, and cell surface receptors are known in the art (See, e.g. Bak and Porteus, Cell Rep. 2017 Jul. 18; 20(3): 750-756 (integration of EGFR); Kanojia et al., Stem Cells. 2015 October; 33(10):2985-94 (expression of anti-Her2 antibody); Eyquem et al., Nature. 2017 Mar. 2; 543(7643):113-117 (site-specific integration of a CAR); O'Connell et al., 2010 PLoS ONE 5(8): e12009 (expression of human IL-7); Tuszynski et al., Nat Med. 2005 May; 11(5):551-5 (expression of NGF in fibroblasts); Sessa et al., Lancet. 2016 Jul. 30; 388(10043):476-87 (expression of arylsulfatase A in ex vivo gene therapy to treat MLD); Rocca et al., Science Translational Medicine 25 Oct. 2017: Vol. 9, Issue 413, eaaj2347 (expression of frataxin); Bak and Porteus, Cell Reports, Vol. 20, Issue 3, 18 Jul. 2017, Pages 750-756 (integrating large transgene cassettes into a single locus), Dever et al., Nature 17 Nov. 2016: 539, 384-389 (adding tNGFR into hematopoietic stem cells (HSC) and HSPCs to select and enrich for modified cells); each of which is herein incorporated by reference in its entirety.

### Homologous Repair Templates

The CYBB cDNA to be integrated, which is comprised by a polynucleotide or donor construct, can be any functional, codon-optimized CYBB cDNA whose expression in cells can restore or improve protein levels in X-CGD patients and thereby allow normal, or clinically beneficial, phagocyte function. The cDNA can be integrated, e.g., at the translational start site of the endogenous CYBB locus, using a template comprising CYBB exons 1-13, or within exon 2, using a template comprising CYBB exons 2-13. In either case, the cDNA is expressed under the control of the endogenous CYBB promoter and other regulatory elements. When exon 2 is targeted, regulatory sequences within the 5′UTR and within intron 1 are retained in particular and can play a role in the expression of the cDNA.

In particular embodiments, the CYBB cDNA in the homologous repair template is codon-optimized, e.g., comprises at least 70%, 75%, 80%, 85%, 90%, 95%, or more homology to the wild-type CYBB cDNA. In a particular embodiment, the CYBB cDNA comprises about 75%, 76%, 77/a, 78/a, 79%, or 80%, homology to the wild-type CYBB cDNA. In a particular embodiment, the CYBB cDNA comprises the codon-optimized sequence shown as SEQ ID NO: 11, or a derivative or fragment of SEQ ID NO:11, e.g., a sequence having about 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.7%, 99.9% or greater identity to SEQ ID NO:11 or to a fragment thereof.

In particular embodiments, the template further comprises a polyA sequence or signal, e.g., a bovine growth hormone polyA sequence or a rabbit beta-globin polyA sequence, at the 3′ end of the cDNA. In particular embodiments, a Woodchuck Hepatitis Virus Posttranscriptional Regulatory Element (WPRE) is included within the 3′UTR of the template, e.g., between the Y end of the CYBB coding sequence and the 5′ end of the polyA sequence, so as to increase the expression of the cDNA. Any suitable WPRE sequence can be used; See, e.g., Zufferey et al. (1999) J. Virol. 73(4):2886-2892; Donello, et al. (1998). J Virol 72: 5085-5092; Loeb, et al. (1999). Hum Gene Ther 10: 2295-2305; the entire disclosures of which are herein incorporated by reference).

In some embodiments, the homologous donor template comprises CYBB exons 1-13 and a bovine growth hormone (bGH) polyA signal (i.e., CYBB1-13-pA). In some embodiments, the homologous donor template comprises CYBB exons 2-13, a bGH polyA signal, and a WPRE element (i.e., CYBB2-13-WPRE-pA). In some embodiments, the donor template comprises CYBB exons 2-13 and a rabbit beta-globin polyA signal (i.e., CYBB2-13-pA).

In particular embodiments, the cDNA (or cDNA and polyA signal) is flanked in the template by CYBB homology regions. For example, an exemplary template can comprise, in linear order: a first CYBB homology region, an CYBB cDNA, a polyA sequence such as a bovine growth hormone polyadenylation sequence (bGH-PolyA) or a rabbit beta-globin polyA sequence, and a second CYBB homology region, where the first and second homology regions are homologous to the genomic sequences extending in either direction from the sgRNA target site. In particular embodiments, one of the homology regions comprises the sequence of nucleotides 1-400 of SEQ ID NO:12, or a contiguous portion thereof, or to a sequence having 80%, 85%, 90%, 95%, 96%, 97%, 98%, 99%, 99.5%, 99.7%, 99.9% or greater identity to nucleotides 1-400 of SEQ ID NO:12, or a contiguous portion thereof. In particular embodiments, the other homology region comprises the sequence of nucleotides 2879-3279 of SEQ ID NO:12, or a contiguous portion thereof, or to a sequence having 80%, 85%, 90%, 95%, 96%, 97%, 98%, 99%, 99.5%, 99.7%, 99.9% or greater identity to nucleotides 2879-3279 of SEQ ID NO:12, or a contiguous portion thereof. The homology regions can be of any size, e.g., 100-1000 bp, 300-800 bp, 400-600 bp, or about 100, 200, 300, 400, 500, 600, 700, 800, 900, 1000, or more bp. In particular embodiments, the homology regions are about 400-500 bp in size.

In particular embodiments, the homologous repair template comprises the sequence shown as SEQ ID NO:12. In other embodiments, the homologous repair template comprises a sequence having 80%, 85%, 90%, 95%, 96% 97%, 98%, 99%, 99.5%, 99.7% 99.9% or greater identity to SEQ ID NO:12, or a fragment thereof.

Any suitable method can be used to introduce the polynucleotide, or donor construct, into the cell. In particular embodiments, the polynucleotide is introduced using a recombinant adeno-associated viral vector, e.g., rAAV6. In some instances, the donor template is single stranded, double stranded, a plasmid or a DNA fragment. In some instances, plasmids comprise elements necessary for replication, including a promoter and optionally a 3′ UTR.

Further disclosed herein are vectors comprising (a) one or more nucleotide sequences homologous to the CYBB locus, and (b) an CYBB cDNA as described herein. The vector can be a viral vector, such as a retroviral, lentiviral (both integration competent and integration defective lentiviral vectors), adenoviral, adeno-associated viral or herpes simplex viral vector. Viral vectors may further comprise genes necessary for replication of the viral vector.

In some embodiments, the targeting construct comprises: (1) a viral vector backbone, e.g. an AAV backbone, to generate virus; (2) arms of homology to the target site of at least 200 bp but ideally at least 400 bp on each side to assure high levels of reproducible targeting to the site (see, Porteus, Annual Review of Pharmacology and Toxicology, Vol. 56:163-190 (2016); which is hereby incorporated by reference in its entirety); (3) a CYBB cDNA encoding a functional protein and capable of expressing the functional protein, a polyA sequence, and optionally a WPRE element; and optionally (4) an additional marker gene to allow for enrichment and/or monitoring of the modified host cells. Any AAV known in the art can be used. In some embodiments the primary AAV serotype is AAV6. In some embodiments, the vector, e.g., rAAV6 vector, comprising the donor template is from about 1-2 kb, 2-3 kb, 3-4 kb, 4-5 kb, 5-6 kb, 6-7 kb, 7-8 kb, or larger.

In some embodiments, viral vectors, e.g., AAV6 vector, is transduced at a multiplicity of infection (MOI) of, e.g., about 1×10³, 5×10³, 1×10⁴, 5×10⁴, 1×10⁵, between 2×10⁴ and 1×10⁵ viruses per cell, or less than 1×10⁵.

Suitable marker genes are known in the art and include Myc, HA, FLAG, GFP, truncated NGFR, truncated EGFR, truncated CD20, truncated CD19, as well as antibiotic resistance genes. In some embodiments, the homologous repair template and/or vector (e.g., AAV6) comprises an expression cassette comprising a coding sequence for truncated nerve growth factor receptor (tNGFR), operably linked to a promoter such as the Ubiquitin C promoter.

In any of the preceding embodiments, the donor template or vector comprises a nucleotide sequence homologous to a fragment of the CYBB locus, optionally to the sequences shown as nucleotides 1-400 and 2879-3279 of SEQ ID NO:12 or contiguous portions thereof, wherein the nucleotide sequence is at least 85%, 88%, 90%, 92%, 95%, 98%, or 99% identical to at least 200, 250, 300, 350, 400, 450, 500, or more consecutive nucleotides of the CYBB locus, e.g., to nucleotides 1-400 and 2879-3279 of SEQ ID NO:12.

The inserted construct can also include other safety switches, such as a standard suicide gene into the locus (e.g. iCasp9) in circumstances where rapid removal of cells might be required due to acute toxicity. The present disclosure provides a robust safety switch so that any engineered cell transplanted into a body can be eliminated, e.g., by removal of an auxotrophic factor. This is especially important if the engineered cell has transformed into a cancerous cell.

The present methods allow for the efficient integration of the donor template at the endogenous CYBB locus. In some embodiments, the present methods allow for the insertion of the donor template in 20%, 25%, 30%, 35%, 40%, or more cells, e.g., cells from an individual with X-CGD. The methods also allow for high levels of expression of gp91phox protein in cells, e.g., cells from an individual with X-CGD, with an integrated CYBB cDNA, e.g., levels of expression that are at least about 70%, 75%, 80%, 85%, 90%, 95%, or more relative to the expression in healthy control cells. The methods also allow for high levels of ROS production (e.g., as measured in a DHR assay) in cells, e.g., cells from an individual with X-CGD, relative to the amount produced in healthy control cells. In some embodiments, the level of ROS produced in an X-CGD cell with an integrated CYBB cDNA as described herein is at least about 70%, 75%, 80%, 85%, 90%, 95%, 100%, or more relative to the level in a healthy control cell.

### 5. Methods of Treatment

Following the integration of the cDNA into the genome of the cell, e.g., HSPC, and confirming expression of the encoded protein, a plurality of modified cells can be reintroduced into the subject, such that they can repopulate and differentiate into, e.g., neutrophils and other phagocytes, and due to the expression of the integrated cDNA, can improve one or more abnormalities or symptoms in the subject with X-CGD. In some embodiments, the cells are expanded, selected, and/or induced to undergo differentiation, prior to reintroduction into the subject.

Disclosed herein, in some embodiments, are methods of treating X-CGD in an individual in need thereof, the method comprising providing to the individual a protein replacement therapy using the genome modification methods disclosed herein. In some instances, the method comprises administering to the individual a host cell that has been modified ex vivo, to comprise a functional CYBB cDNA, integrated at the CYBB locus, wherein said modified host cell expresses the encoded protein which is otherwise deficient in the individual, thereby treating the X-CGD in the individual.

### Pharmaceutical Compositions

Disclosed herein, in some embodiments, are methods, compositions and kits for use of the modified cells, including pharmaceutical compositions, therapeutic methods, and methods of administration. Although the descriptions of pharmaceutical compositions provided herein are principally directed to pharmaceutical compositions which are suitable for administration to humans, it will be understood by the skilled artisan that such compositions are generally suitable for administration to any animals. In some embodiments, the modified cells of the pharmaceutical composition are autologous to the individual in need thereof. In other embodiments, the modified cells of the pharmaceutical composition are allogeneic to the individual in need thereof.

In some embodiments, a pharmaceutical composition comprising a modified host cell as described herein is provided. The modified host cell is genetically engineered to comprise an integrated CYBB cDNA at the CYBB locus. In particular embodiments, a functional codon-optimized full-length CYBB cDNA (comprising exons 1-13) is integrated into the translational start site of the endogenous CYBB locus. In other particular embodiments, a functional codon-optimized partial CYBB cDNA (comprising exons 2-13) is integrated into exon 2 of the endogenous CYBB locus. In particular embodiments, the functional codon-optimized CYBB cDNA that is integrated into the host cell genome is expressed under control of the native CYBB promoter sequence. In some embodiments, the pharmaceutical composition comprises a plurality of the modified host cells, and further comprises unmodified host cells and/or host cells that have undergone nuclease cleavage resulting in INDELS at the CYBB locus but not integration of the CYBB cDNA. In some embodiments, the pharmaceutical composition is comprised of at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50% or more of the modified host cells comprising an integrated IL2RG cDNA. The pharmaceutical compositions described herein may be formulated using one or more excipients to, e.g.: (1) increase stability; (2) alter the biodistribution (e.g., target the cells to specific tissues or cell types); (3) alter the release profile of an encoded therapeutic factor.

Formulations of the present disclosure can include, without limitation, saline, liposomes, lipid nanoparticles, polymers, peptides, proteins, and combinations thereof. Formulations of the pharmaceutical compositions described herein may be prepared by any method known or hereafter developed in the art of pharmacology. As used herein the term “pharmaceutical composition” refers to compositions including at least one active ingredient (e.g., a modified host cell) and optionally one or more pharmaceutically acceptable excipients. Pharmaceutical compositions of the present disclosure may be sterile.

Relative amounts of the active ingredient (e.g., the modified host cell), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%. e.g., between 0.5 and 50%, between 1-30%, between 5-80%, or at least 80% (w/w) active ingredient.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, M D, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

### Dosing and Administration

The modified host cells of the present disclosure included in the pharmaceutical compositions described above may be administered by any delivery route, systemic delivery or local delivery, which results in a therapeutically effective outcome. These include, but are not limited to, enteral, gastroenteral, epidural, oral, transdermal, intracerebral, intracerebroventricular, epicutaneous, intradermal, subcutaneous, nasal, intravenous, intra-arterial, intramuscular, intracardiac, intraosseous, intrathecal, intraparenchymal, intraperitoneal, intravesical, intravitreal, intracavernous), interstitial, intra-abdominal, intralymphatic, intramedullary, intrapulmonary, intraspinal, intrasynovial, intrathecal, intratubular, parenteral, percutaneous, periarticular, peridural, perineural, periodontal, rectal, soft tissue, and topical. In particular embodiments, the cells are administered intravenously. In certain embodiments, the composition may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

In some embodiments, a subject will undergo a conditioning regime before cell transplantation. For example, before hematopoietic stem cell transplantation, a subject may undergo myeloablative therapy, non-myeloablative therapy or reduced intensity conditioning to prevent rejection of the stem cell transplant even if the stem cell originated from the same subject. The conditioning regime may involve administration of cytotoxic agents. The conditioning regime may also include immunosuppression, antibodies, and irradiation. Other possible conditioning regimens include antibody-mediated conditioning (see, e.g., Czechowicz et al., 318(5854) Science 1296-9 (2007); Palchaudari et al., 34(7) Nature Biotechnology 738-745 (2016); Chhabra et al., 10:8(351) Science Translational Medicine 351ra105 (2016)) and CAR T-mediated conditioning (see, e.g., Arai et al., 26(5) Molecular Therapy 1181-1197 (2018); each of which is hereby incorporated by reference in its entirety). For example, conditioning needs to be used to create space in the brain for microglia derived from engineered hematopoietic stem cells (HSCs) to migrate in to deliver the protein of interest (as in recent gene therapy trials for ALD and MLD). The conditioning regimen is also designed to create niche “space” to allow the transplanted cells to have a place in the body to engraft and proliferate. In HSC transplantation, for example, the conditioning regimen creates niche space in the bone marrow for the transplanted HSCs to engraft. Without a conditioning regimen, the transplanted HSCs cannot engraft.

Certain aspects of the present disclosure are directed to methods of providing pharmaceutical compositions including the modified host cell of the present disclosure to target tissues of mammalian subjects, by contacting target tissues with pharmaceutical compositions including the modified host cell under conditions such that they are substantially retained in such target tissues. In some embodiments, pharmaceutical compositions including the modified host cell include one or more cell penetration agents, although “naked” formulations (such as without cell penetration agents or other agents) are also contemplated, with or without pharmaceutically acceptable excipients.

The present disclosure additionally provides methods of administering modified host cells in accordance with the disclosure to a subject in need thereof. The pharmaceutical compositions including the modified host cell, and compositions of the present disclosure may be administered to a subject using any amount and any route of administration effective for preventing, treating, or managing the X-CGD. The exact amount required will vary from subject to subject, depending on the species, age, and general condition of the subject, the severity of the disease, the particular composition, its mode of administration, its mode of activity, and the like. The subject may be a human, a mammal, or an animal. The specific therapeutically or prophylactically effective dose level for any particular individual will depend upon a variety of factors including the disorder being treated and the severity of the disorder; the activity of the specific payload employed; the specific composition employed; the age, body weight, general health, sex and diet of the patient; the time of administration, route of administration; the duration of the treatment; drugs used in combination or coincidental with the specific modified host cell employed; and like factors well known in the medical arts.

In certain embodiments, modified host cell pharmaceutical compositions in accordance with the present disclosure may be administered at dosage levels sufficient to deliver from, e.g., about 1×10⁴ to 1×10⁵, 1×10⁵ to 1×10⁶, 1×10⁶ to 1×10⁷, or more cells to the subject, or any amount sufficient to obtain the desired therapeutic or prophylactic, effect. The desired dosage of the modified host cell pharmaceutical compositions of the present disclosure may be administered one time or multiple times. In some embodiments, delivery of the modified host cell to a subject provides a therapeutic effect for at least 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years.

The modified host cells may be used in combination with one or more other therapeutic, prophylactic, research or diagnostic agents, or medical procedures, either sequentially or concurrently. In general, each agent will be administered at a dose and/or on a time schedule determined for that agent.

Use of a modified mammalian host cell according to the present disclosure for treatment of X-CGD is also encompassed by the disclosure.

The present disclosure also contemplates kits comprising compositions or components as described herein, e.g., sgRNA, Cas9, RNPs, i53 mRNA, and/or homologous templates, as well as, optionally, reagents for, e.g., the introduction of the components into cells. The kits can also comprise one or more containers or vials, as well as instructions for using the compositions in order to modify cells and treat subjects according to the methods described herein.

### 6. Examples

The present disclosure will be described in greater detail by way of specific examples. The following examples are offered for illustrative purposes only, and are not intended to limit the disclosure in any manner. Those of skill in the art will readily recognize a variety of noncritical parameters which can be changed or modified to yield essentially the same results.

### Example 1. Correction of X-CGD Patient Hematopoietic Stem/Progenitor Cells by Targeted CYBB cDNA Insertion Using CRISPR/Cas9 with 53BP1 Inhibition for Enhanced Homology-Directed Repair

### Abstract

X-linked chronic granulomatous disease is an immunodeficiency characterized by defective production of microbicidal reactive oxygen species (ROS) by phagocytes. Causative mutations occur throughout the 13 exons and splice sites of the CYBB gene, resulting in loss of gp91^{phox} protein. Here we report gene correction by homology-directed repair in patient hematopoietic stem/progenitor cells (HSPCs) using CRISPR/Cas9 for targeted insertion of CYBB exon 1-13 or 2-13 cDNAs from adeno-associated virus donors at endogenous CYBB exon 1 or exon 2 sites. Targeted insertion of exon 1-13 cDNA did not restore physiologic gp91^{phox} levels, consistent with a requirement for intron 1 in CYBB expression. However, insertion of exon 2-13 cDNA fully restored gp91^{phox} and ROS production upon phagocyte differentiation. Addition of a woodchuck hepatitis virus post-transcriptional regulatory element did not further enhance gp91^{phox} expression in exon 2-13 corrected cells, indicating that retention of intron 1 was sufficient for optimal CYBB expression. Targeted correction was increased ˜˜1.5-fold using i53 mRNA to transiently inhibit non-homologous end joining. Following engraftment in NSG mice, corrected HSPCs generated phagocytes with restored gp91^{phox} and ROS production. Our findings demonstrate the utility of tailoring donor design and targeting strategies to retain regulatory elements needed for optimal expression of the target gene.

### Introduction

We previously described (21) a gene insertion approach for correction of X-CGD patient HSPCs using ZFNs targeting the “safe-harbor” AAVS1 locus (14) to mediate insertion of a codon-optimized full-length CYBB cDNA under the control of a constitutive promoter, using an AAV6 vector to deliver the donor DNA template for HDR. This safe-harbor targeted insertion strategy resulted in the constitutive expression of gp91^{phox} protein in HSPC-derived phagocytes at per-cell levels that were ˜60% of levels in healthy donor controls, which restored ROS production in HSPC-derived phagocytes to per-cell levels ˜90% of healthy controls, albeit with the potential risks associated with dysregulated constitutive gp91^{phox} expression mentioned above. As an alternate strategy for correction of a specific point mutation while maintaining normal physiological regulation of gene expression, we also described (27) the targeted repair of a C676T mutation in CYBB exon 7 in affected X-CGD patient HSPCs by CRISPR/Cas9 using a mutation-specific CRISPR single guide RNA (sgRNA) and a short ssODN donor template to correct the point mutation at this site by HDR. Gene correction in HSPCs by this approach resulted in the restoration of physiologically normal levels of gp91^{phox} protein expression and ROS production in HSPC-derived phagocytes, demonstrating an effective and efficient gene repair strategy for the cohort of patients that share this specific point mutation.

Here we describe the further application of these strategies for efficient cDNA insertion into the CYBB locus to achieve gene correction in X-CGD patient CD34⁺ HSPCs using AAV6 donor templates to deliver CYBB exon 1-13 or exon 2-13 cDNAs, both to confirm the necessity of CYBB intron 1 for cDNA expression from the CYBB promoter in corrected primary hematopoietic cells and to extend these strategies to efficient correction of patient HSPCs as a relevant cell type for clinical gene therapy. We also investigate the effect of inhibition of the NHEJ repair pathway by transient expression of an mRNA encoding i53 (38), a recently described inhibitor of the NHEJ-promoting DNA repair protein 53BP1 (39), in order to enhance the efficiency of HDR-mediated gene correction in HSPCs.

### Results

Targeted CYBB cDNA Insertion into CYBB Exon 1 Or Exon 2

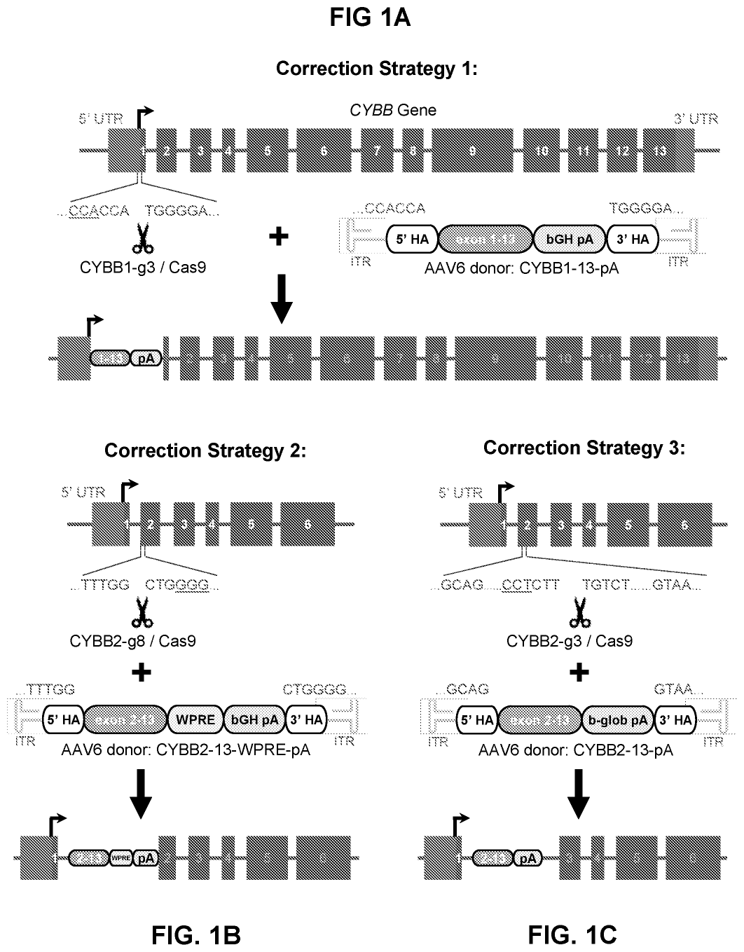

We previously reported that the targeted insertion of a full-length CYBB exon 1-13 cDNA plus a poly-adenylation signal (poly-A) into the start site of the endogenous CYBB exon 1 in X-CGD patient iPSCs failed to restore gp91^{phox} expression upon phagocyte differentiation, apparently due to the elimination of critical sequences in intron 1 necessary for expression from the CYBB promoter (36). To determine whether intronic elements are likewise required for cDNA expression from the CYBB promoter in adult somatic HSPCs, we assessed HDR-mediated targeted insertion of a codon-optimized full-length CYBB exon 1-13 cDNA (40) and a bovine growth hormone (bGH) poly-A to the beginning of the endogenous CYBB exon 1 in X-CGD HSPCs, using a CRISPR/Cas9 sgRNA (CYBB1-g3) and an AAV6 donor template containing homology arms flanking the endogenous CRISPR/Cas9 cut site (FIG. 1A), referred to here as correction strategy 1. We also assessed two additional strategies (correction strategies 2 and 3) for targeted insertion of codon-optimized CYBB exon 2-13 cDNAs (36, 40) into exon 2 of the CYBB locus, thereby retaining the endogenous intron 1 in the resulting transcript. Correction strategy 2 utilized a CRISPR/Cas9 sgRNA (CYBB2-g8) targeting the beginning region of the endogenous exon 2 for targeted insertion of a codon-optimized exon 2-13 cDNA (40) together with a bGH poly-A (41) and a woodchuck hepatitis virus post-transcriptional regulatory element (WPRE) (42-44), using an AAV6 donor template containing homology arms flanking the endogenous cut site (FIG. 1B). Correction strategy 3 utilized a CRISPR/Cas9 sgRNA (CYBB2-g3) targeting further downstream within exon 2 for replacement of the entire endogenous exon 2 sequence with a codon-optimized exon 2-13 cDNA (36) and a strong poly-A signal derived from the rabbit beta-globin gene (including downstream helper sequences for more efficient transcription termination) (45, 46), using an AAV6 donor template containing a 5′ homology arm corresponding to intron 1 sequences ending at the splice acceptor site located 32 nucleotides upstream from the Cas9 cut site and a 3′ homology arm corresponding to intron 2 sequences beginning at the splice donor site located 64 nucleotides downstream from the Cas9 cut site (FIG. 1C).

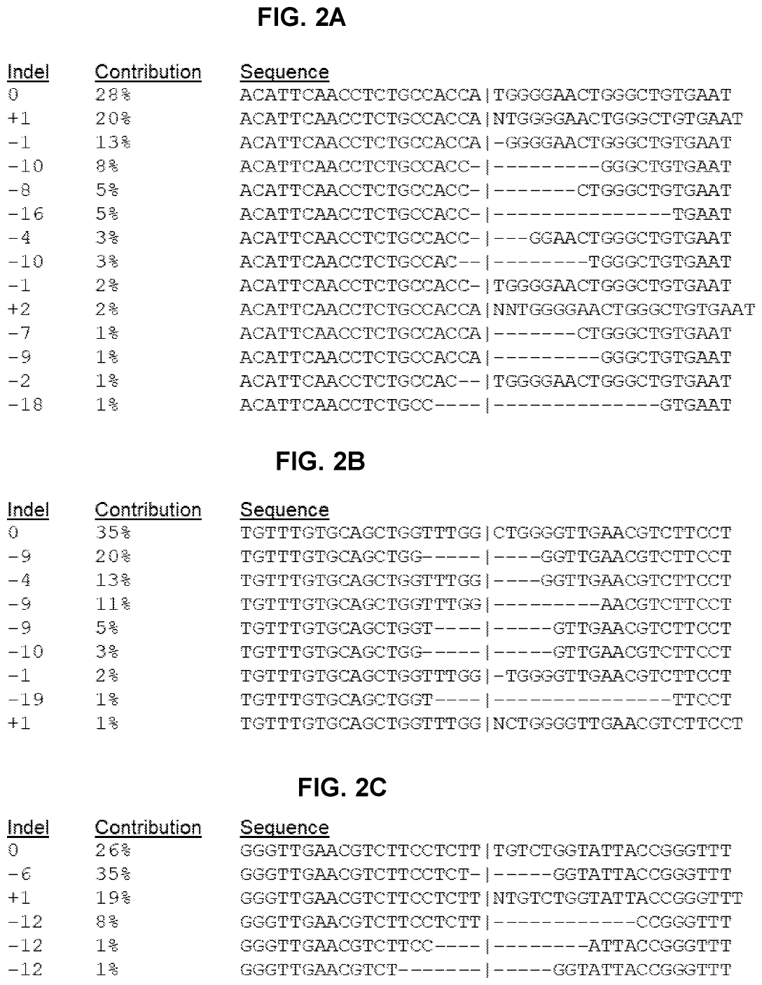

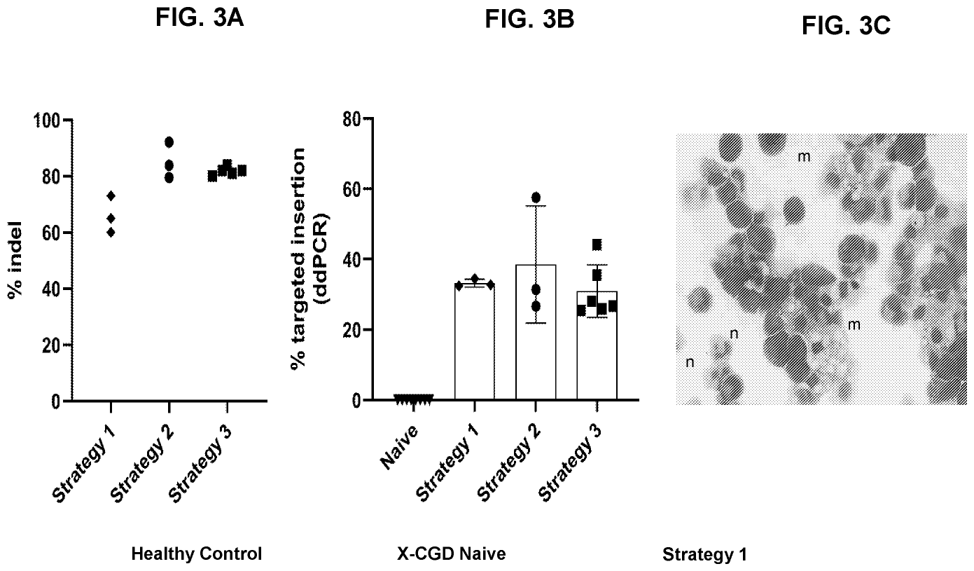

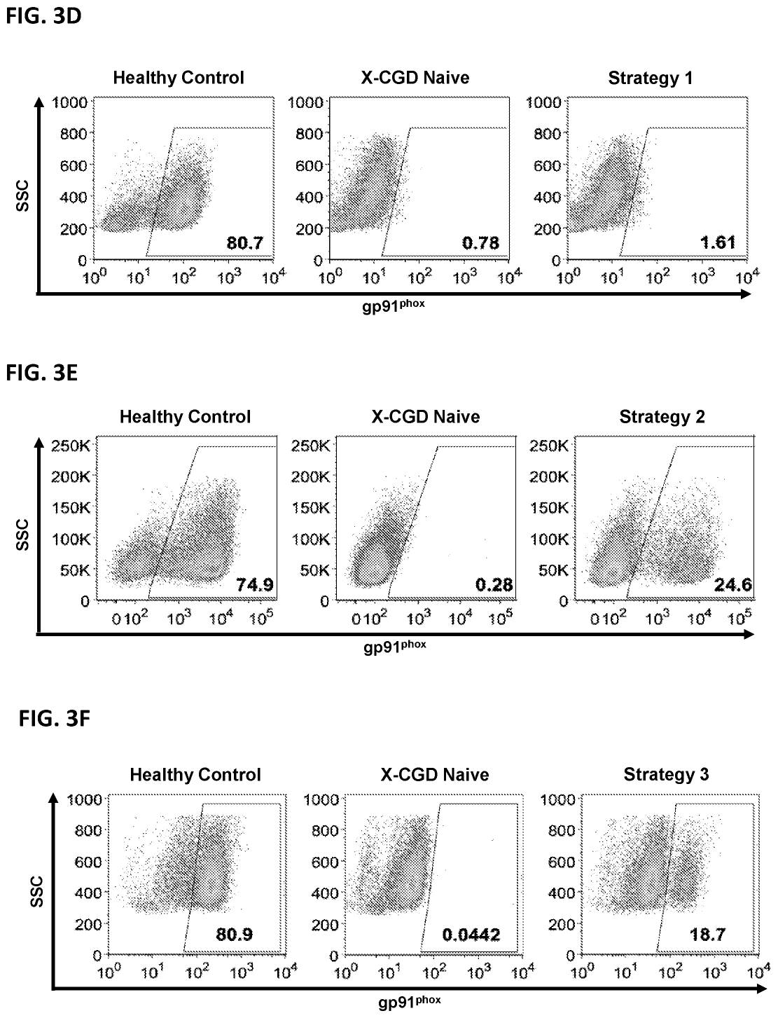

The cutting activity of the CRISPR/Cas9 CYBB1-g3 sgRNA targeting CYBB exon 1 for strategy 1 was 65% (median) by ICE analysis of indel formation (FIGS. 2A and 3A), and targeted insertion of the full-length CYBB exon 1-13 cDNA could be detected by droplet digital PCR (ddPCR) analysis in 33% (mean) of patient HSPCs (FIG. 3B). However, upon in vitro differentiation of HSPCs into phagocytes (FIG. 3C), there was little or no detectable gp91^{phox} protein expression above background levels (FIG. 3D), consistent with our previous findings in iPSCs. The CRISPR/Cas9 CYBB2-g8 and CYBB2-g3 sgRNAs targeting CYBB exon 2 for strategies 2 and 3 exhibited similar cutting activities (median of 84% and 82%, respectively), based on analysis of indel formation (FIGS. 2B, 2C, and 3A), resulting in similar targeted insertion efficiencies (mean of 39% and 31%, respectively) by ddPCR analysis (FIG. 3B). However, in contrast to strategy 1, correction of X-CGD patient HSPCs by either strategy 2 or strategy 3 resulted in substantial restoration of gp91^{phox} expression upon phagocyte differentiation (FIGS. 3E and 3F), consistent with a required role for intron 1 sequences in expression from the endogenous CYBB promoter. Notably, in vitro phagocyte differentiation of healthy control CD34⁺ HSPCs resulted in only ˜80% gp91^{phox+} cells, indicating incomplete differentiation of the total HSPC population into mature phagocytes under these culture conditions for gp91^{phox} expression; consequently, measurements of targeted correction efficiencies based on % gp91^{phox+} phagocytes appear correspondingly lower than the targeted correction efficiencies measured by ddPCR analysis.

### Increased Targeted Insertion by 53BP1 Inhibition

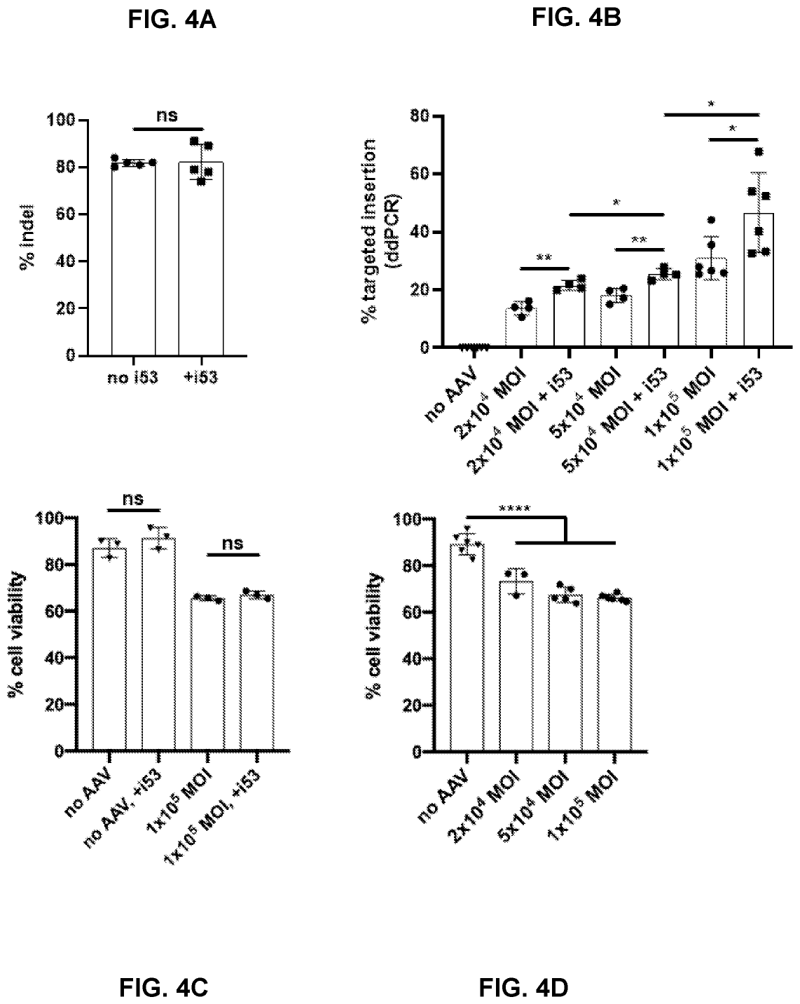

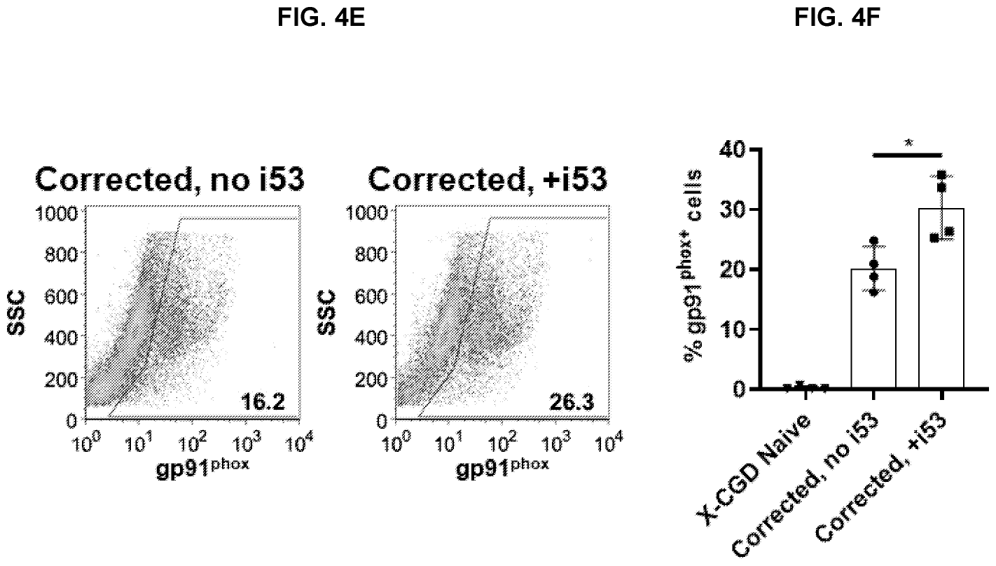

For X-CGD, healthy or gene corrected phagocytes do not possess a survival or proliferative advantage over diseased ones, therefore a high frequency of genome editing is necessary in order to achieve a clinically beneficial threshold of functional correction (8, 47). In order to optimize HDR-mediated targeted insertion efficiencies of exon 2-13 cDNA constructs in X-CGD HSPCs, we investigated the effects of transient inhibition of 53BP1 in the NHEJ pathway using i53 mRNA (38). Enhancement of HDR by i53 for targeted insertion had previously been reported in immortalized human cell lines (38, 48-50), but not in primary human cells. HSPCs from healthy male donors were used for our initial optimization studies, to model the mono-allelic CYBB target of male X-CGD patients. Inclusion of i53 mRNA with CRISPR/Cas9 ribonucleoprotein (RNP) complexes during electroporation of HSPCs did not alter the overall frequency of indel formation at the CYBB target site in the absence of AAV6 donor template (FIG. 4A). However, when combined with AAV6 transduction at a multiplicity of infection (MOI) ranging between 2×10⁴ and 1×10⁵ viruses per cell, i53 resulted in approximately 1.5-fold increased targeted insertion of AAV6 donor template in HSPCs at all MOIs tested (FIG. 4B) based on ddPCR analysis, with higher insertion frequencies evident with increasing MOIs. Transient expression of i53 had no significant effect on the viability of transfected HSPCs either in the presence or absence of AAV6 transduction (FIG. 4C), however AAV6 itself significantly reduced HSPC viability (FIGS. 4C and 4D), with an apparent trend towards decreasing viability at higher MOIs; consequently. MOIs>1×10⁵ were not used in our correction studies. The enhanced targeted insertion efficiency in HSPCs achieved using i53 likewise resulted in an approximately 1.5-fold increase in the percentage of gp91^{phox} expressing phagocytes upon differentiation from corrected X-CGD patient HSPCs (FIGS. 4E and 4F); the relatively lower degree of functional correction based on gp91^{phox} expression (FIGS. 4E and 4F) compared to ddPCR analysis (FIG. 4B) again reflects the incomplete differentiation into mature phagocytes necessary for gp91^{phox} expression under these culture conditions, as noted above.

Comparison of Correction Strategies for gp91^{phox} Expression and ROS Production

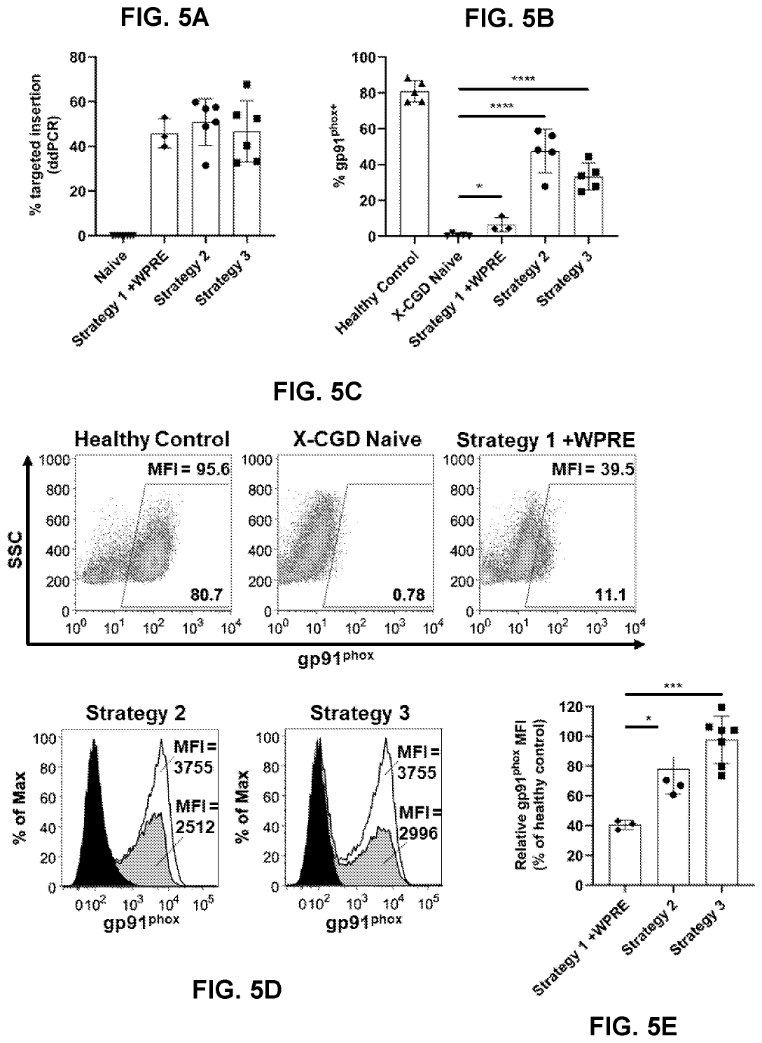

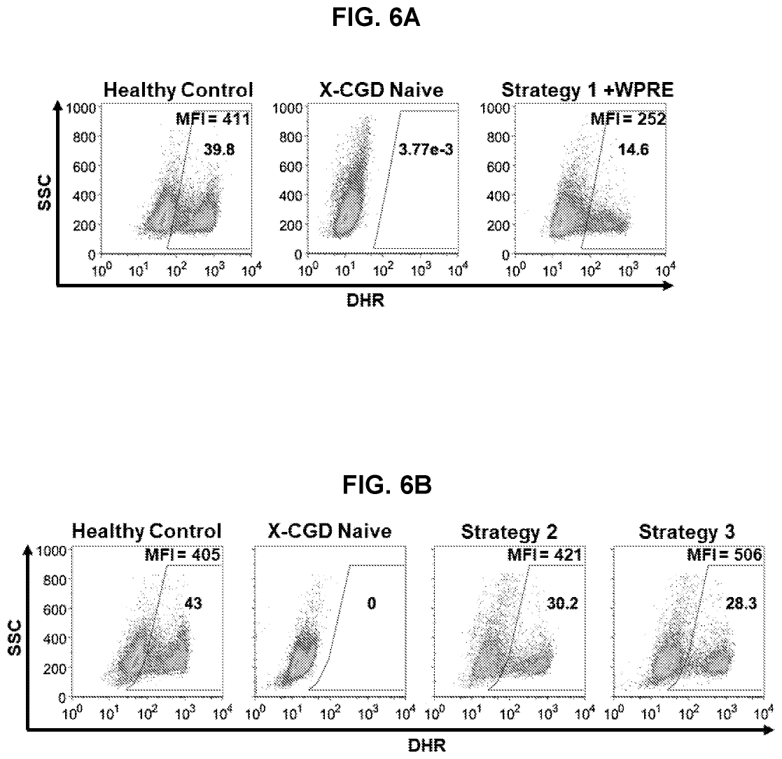

To further assess the necessity of CYBB intron 1 and the effects of a WPRE on expression of inserted CYBB cDNAs, we tested an alteration to correction strategy 1 using a modified AAV6 donor template containing a WPRE in addition to the exon 1-13 cDNA and bGH poly-A (termed “strategy 1+WPRE”), in comparison with strategies 2 and 3. Using optimized targeted insertion conditions, all three strategies resulted in highly efficient targeted insertion in HSPCs based on ddPCR analysis (FIG. 5A), resulting in significant restoration of gp91^{phox} expression in phagocytes differentiated from corrected X-CGD patient HSPCs (FIG. 5B). The inclusion of a WPRE with the exon 1-13 cDNA resulted in a partial increase in gp91^{phox} expression (FIG. 5C versus FIG. 3D), but did not fully restore the per-cell expression levels to those detected in healthy control cells, based on the relative mean fluorescence intensities (MFIs) of gp91^{phox+} gated cells by flow cytometry immunostaining. For X-CGD HSPCs corrected with strategy 2, the population of corrected phagocytes following in vitro differentiation exhibited mean per-cell levels of gp91^{phox} expression that were 78% of levels in gp91^{phox+} healthy donor controls, and correction with strategy 3 resulted in mean per-cell levels that were 98% of healthy control levels (FIGS. 5D and 5E), compared to 41% for strategy 1+WPRE (FIGS. 5C and 5E). This indicates that while the WPRE could partially restore expression of exon 1-13 cDNA in the absence of intron 1, the targeted insertion of exon 2-13 cDNA with a strong poly-A signal (45,46) was sufficient for achieving physiologically normal levels of gp91^{phox} protein expression when intron 1 was retained, without requiring additional exogenous regulatory elements such as a WPRE. Targeted insertion using strategy 1+WPRE resulted in partial functional restoration of per-cell levels of ROS production in phagocytes, compared to levels detected in healthy donor controls (FIG. 6A), while both strategy 2 and 3 resulted in restoration of per-cell levels of ROS production to physiologically normal levels (FIG. 6B), based on the MFIs of positive gated cells by dihydrorhodamine-123 (DHR) assay. The lower cell population percentages of DHR⁺ cells compared to gp91^{phox+} cells (FIG. 6 versus FIG. 5B) for both healthy control and corrected X-CGD patient cells reflect the varying degrees of phagocyte maturation required for gp91^{phox} protein expression versus functional ROS production, and again are consistent with incomplete phagocyte differentiation and maturation of the total populations under these culture conditions.

### In Vivo Engraftment of Gene Corrected HSPCs

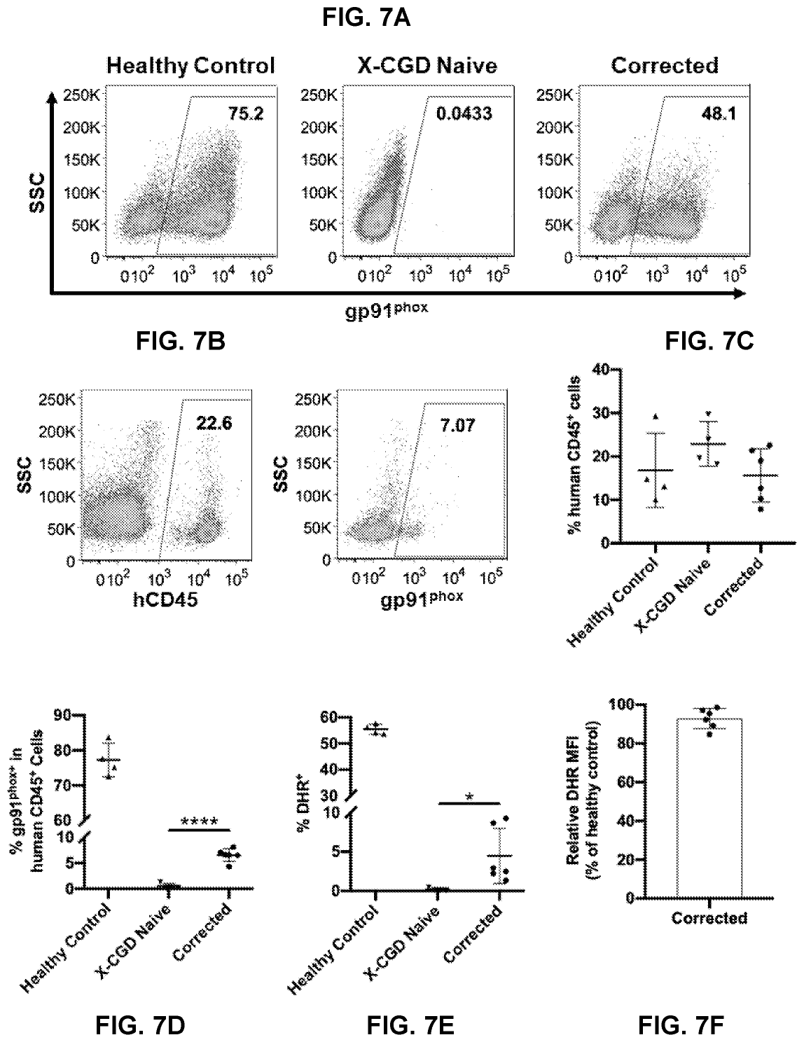

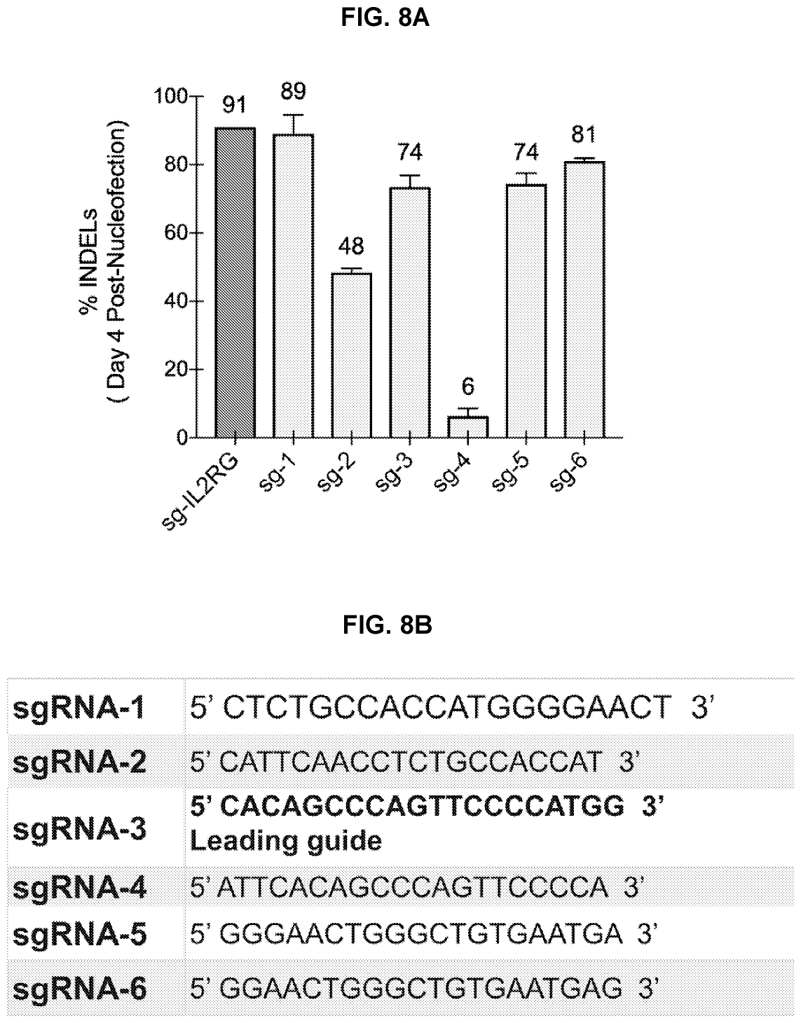

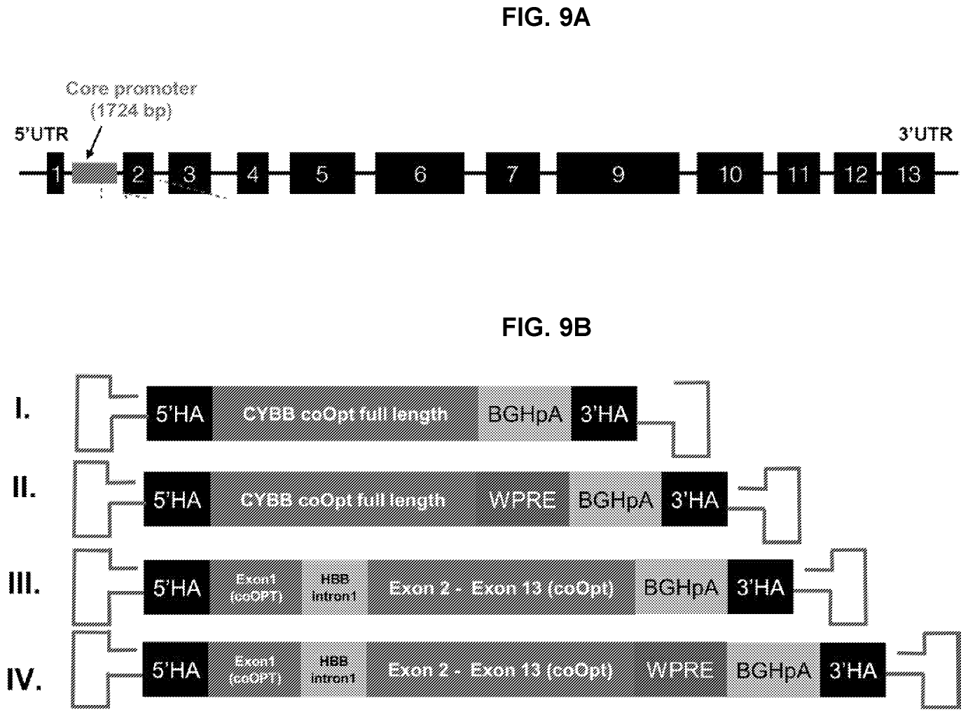

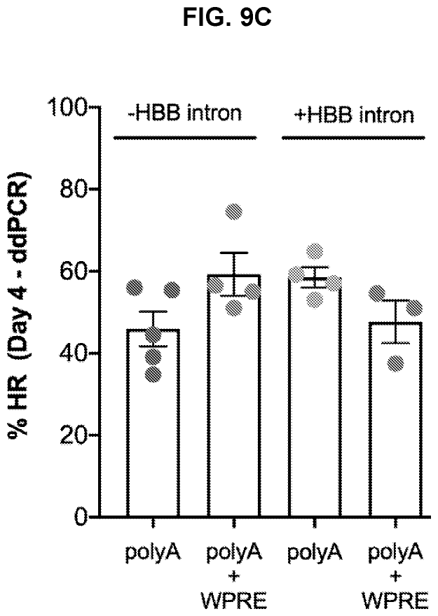

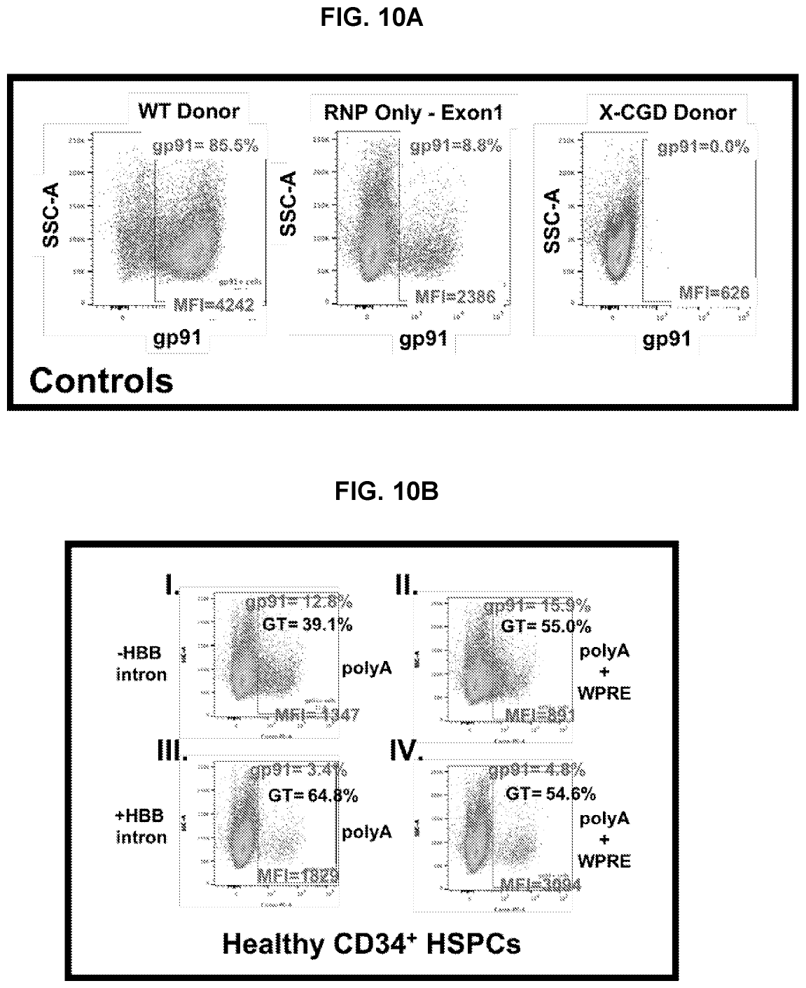

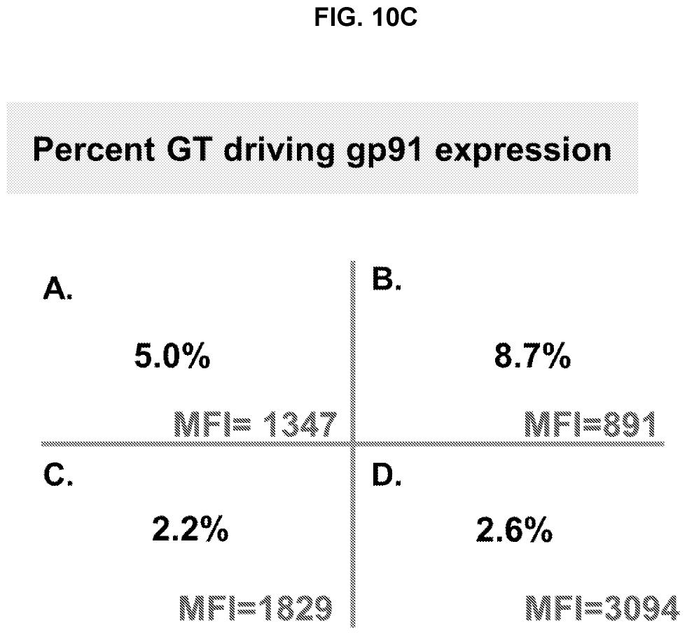

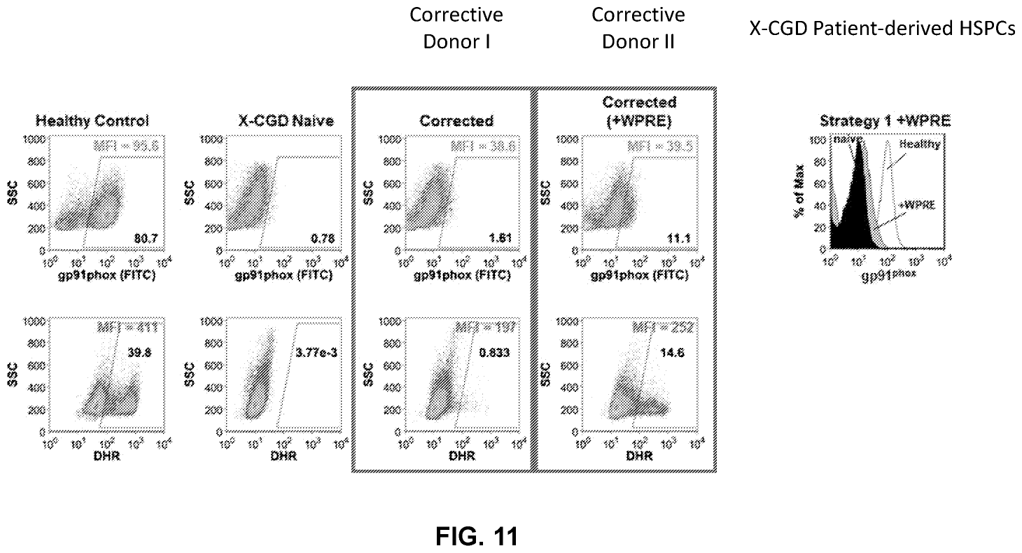

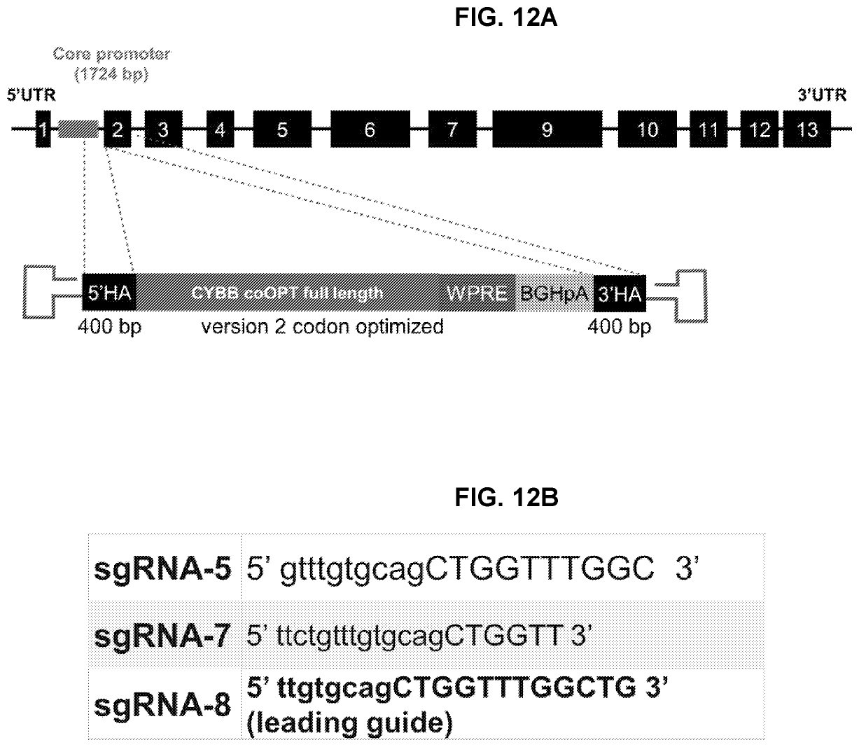

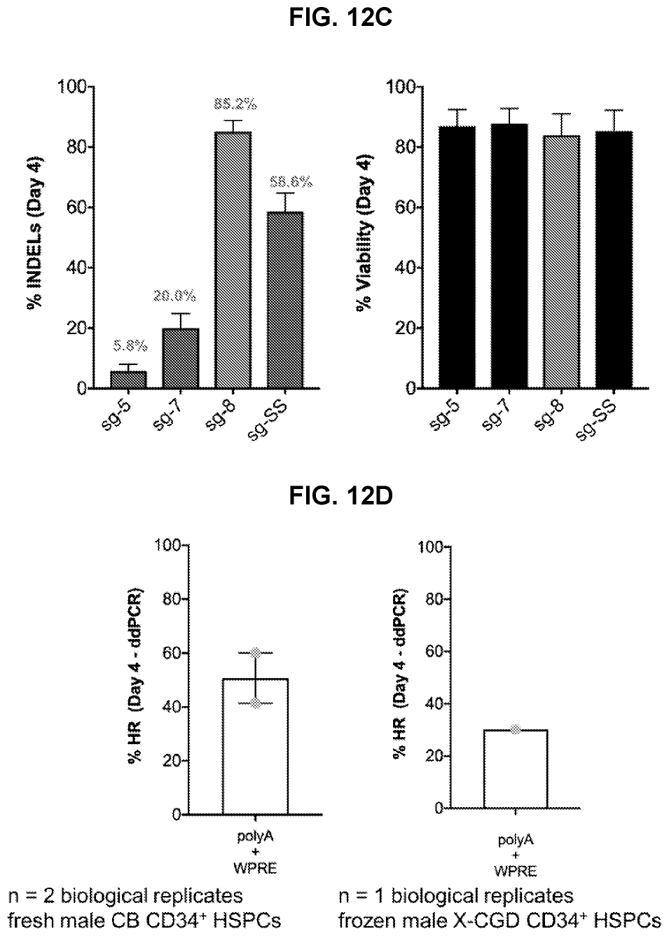

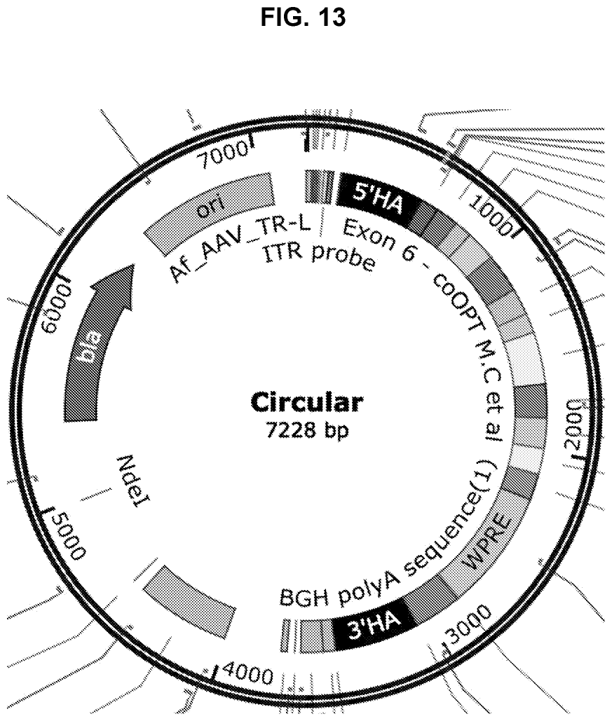

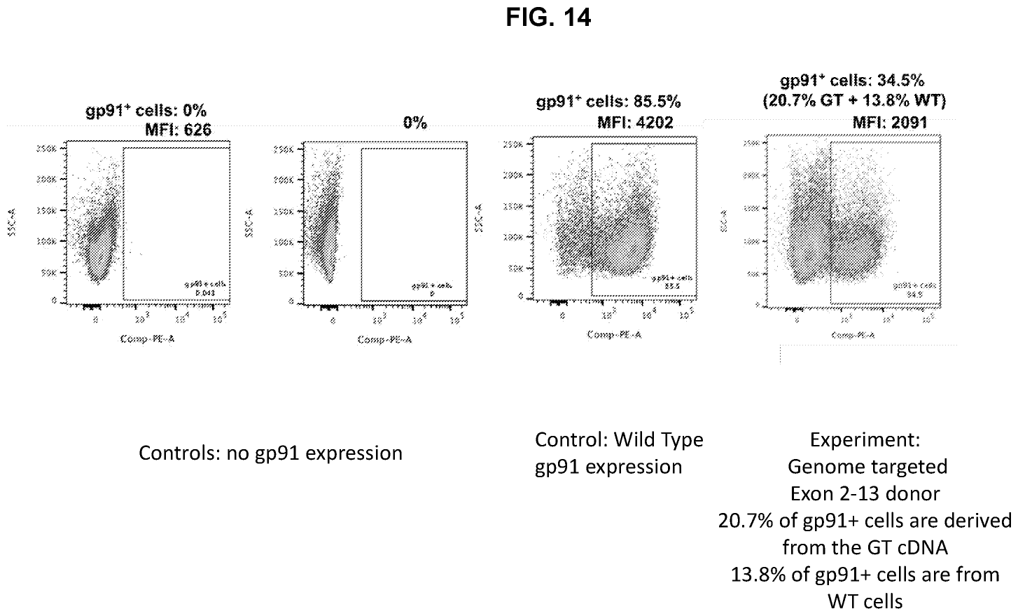

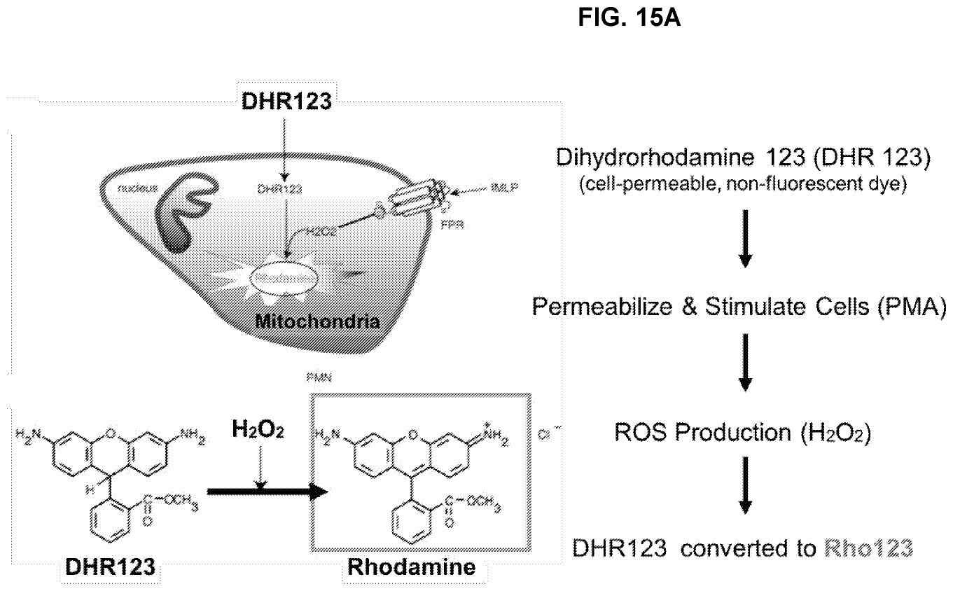

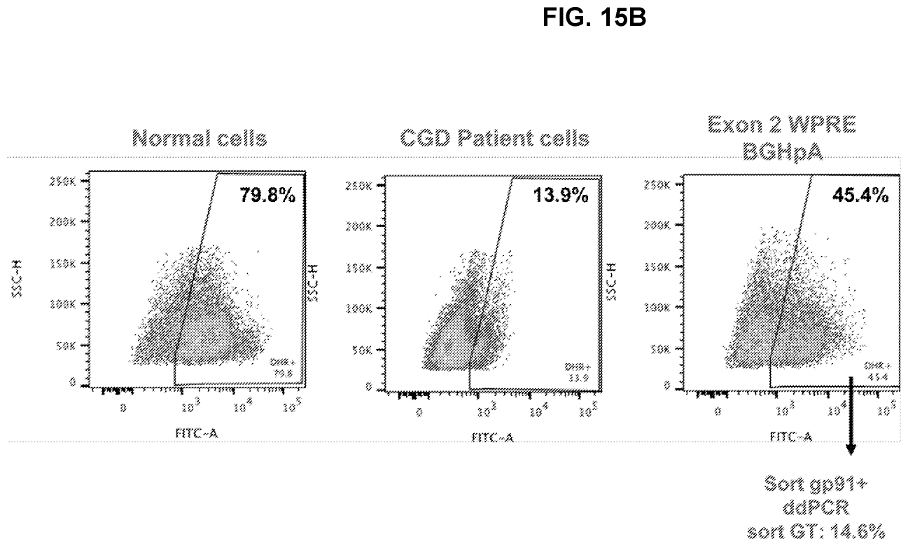

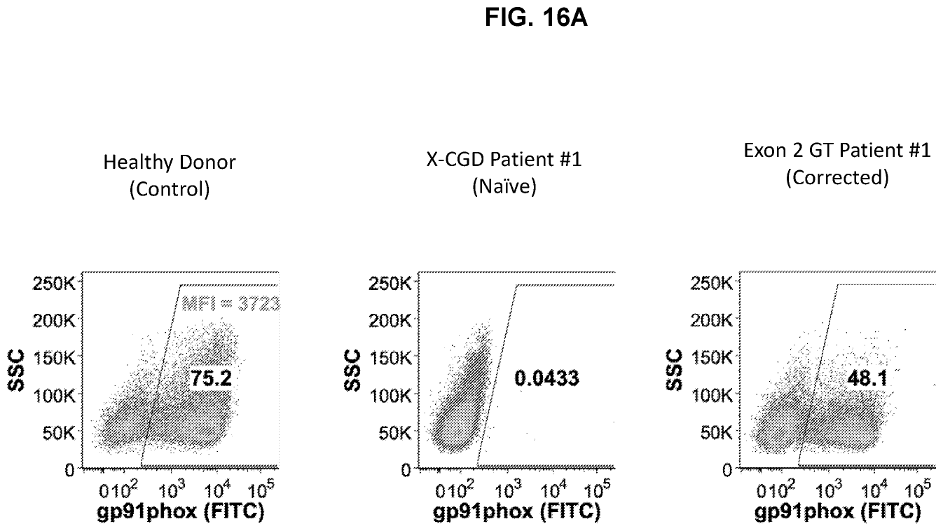

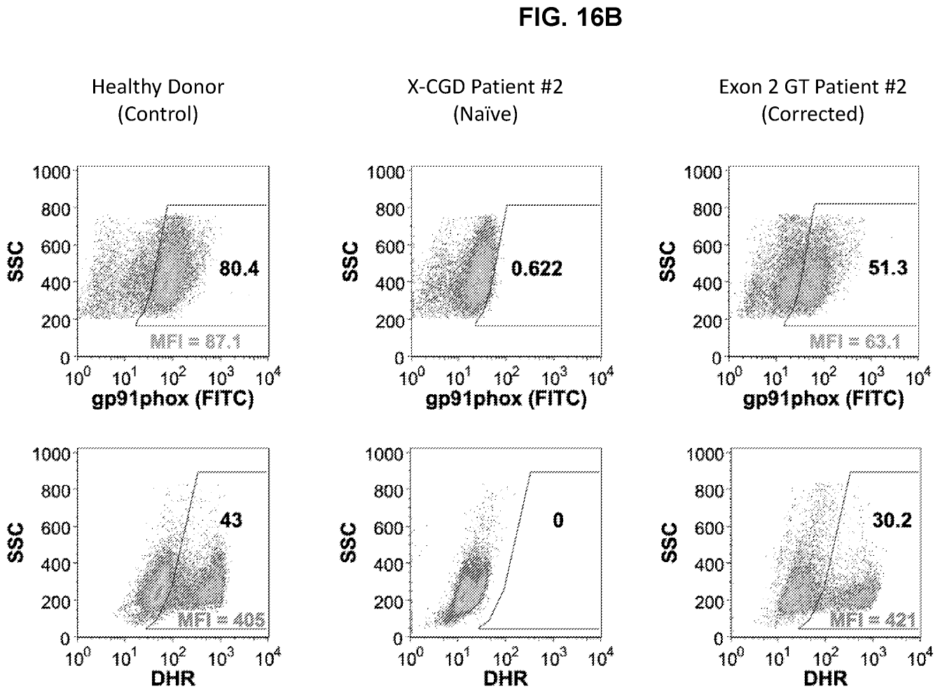

In order to assess engraftment potential following gene correction, X-CGD patient HSPCs were corrected under optimized targeted insertion conditions with CYBB exon 2-13 cDNA insertion using strategy 2, and were then transplanted into busulfan-preconditioned immunodeficient NOD.Cg-Prkdc^{scid} CYBB^{tm1Wj1}/SzJ (NSG) mice. The pre-transplant HSPC population exhibited 60% targeted correction by ddPCR analysis, and 48% of cells expressed gp91^{phox} upon in vitro phagocyte differentiation (FIG. 7A). Substantial human cell engraftment was detected in peripheral blood at 12 weeks post-transplant based on human CD45 immunostaining, similar to transplanted naive X-CGD or healthy donor control HSPCs (FIGS. 7B and 7C). A subset of the engrafted corrected X-CGD cells exhibited restored gp91^{phox} expression in vivo (FIGS. 7B and 7D), although at lower marking frequencies than in the input HSPC population used for transplant (mean value of 7.6% of engrafted human CD45⁺ cells were gp91^{phox+} in FIG. 7D versus ˜48% gp91^{phox+} for in vitro differentiated phagocytes in FIG. 7A), while healthy donor control cells exhibited similar gp91^{phox} population percentages post-transplant as in the pre-transplant population (FIG. 7D versus FIG. 7A). Mouse marrow was harvested at 16 weeks post-transplant, from which engrafted human CD34⁺ HSPCs were sorted and then differentiated in vitro to produce mature neutrophils that exhibited functional restoration of ROS production (FIG. 7E; mean of 4.5% DHR⁺ phagocytes) at mean per-cell levels that were 93% of levels in healthy control neutrophils (FIG. 7F), confirming that a population of gene corrected CD34⁺ HSPCs was maintained in vivo following engraftment, albeit with decreased percentages of functionally corrected cells than the level of targeted gene insertion initially detected in the pre-transplant HSPC population, consistent with the gp91^{phox} expression data.

### Discussion

Here we demonstrate several strategies for HDR-mediated targeted insertion of CYBB cDNAs from AAV6-based donor DNA templates into the CYBB locus in X-CGD patient HSPCs using CRISPR/Cas9, in order to achieve regulated expression of the inserted cDNA by the endogenous CYBB promoter for functional correction of patient cells. Targeted insertion of an intronless full-length CYBB exon 1-13 cDNA and a poly-A signal into the start site of exon 1 of the CYBB locus could be achieved efficiently but resulted in little or no detectable expression of gp91^{phox} protein upon phagocyte differentiation, possibly due to the elimination of one or more important transcriptional regulatory elements in CYBB intron 1, such as a putative NF-κB enhancer element that normally interacts with a distant upstream NF-κB binding site and with the CYBB promoter to regulate CYBB expression in phagocytes (51). The lack of detectable gp91^{phox} expression after targeted insertion of exon 1-13 cDNA is consistent with our previous findings in X-CGD patient iPSCs (36), validating the use of iPSCs for modeling the efficacy of targeted insertion strategies for functional correction and expression in relevant cell lineages. Inclusion of a WPRE with the exon 1-13 cDNA partially restored gp91^{phox} expression in the present study, but was unable to completely compensate for the loss of intron 1. A similar gene editing strategy targeting the exon 1 start site of the CD40LG gene for full-length cDNA insertion in T cells of patients with X-linked hyper-IgM syndrome was shown by Hubbard et al. (35) to fully restore normal physiological levels of CD40L protein expression and its functional activity, additionally, insertion of an intronless full-length CYBB exon 1-13 cDNA under the control of an exogenous promoter using safe-harbor targeted insertion (14) or using retroviral or lentiviral vectors (3, 4, 7, 8, 52) in X-CGD patient HSPCs has previously been shown to result in substantial gp91^{phox} expression in phagocytes, in contrast to our present data regarding intronless full-length cDNA expression from the CYBB promoter. These findings reflect the disparity in the regulation of expression between different genes and promoters, which should be taken into consideration when developing gene editing strategies.

As an alternative approach for correction of CYBB mutations occurring downstream of intron 1 (encompassing ˜90% of reported X-CGD patients) (1), we also tested the targeted insertion of either of two CYBB exon 2-13 cDNA constructs (differing in the presence or absence of a WPRE as an exogenous regulatory element for enhanced expression) and a poly-A signal into exon 2 of the CYBB locus, to retain the endogenous CYBB intron 1 in the resulting transcript. Efficient targeted correction of X-CGD patient HSPCs could be achieved with either exon 2-13 cDNA construct, restoring gp91^{phox} protein expression and ROS production in phagocytes derived from gene corrected HSPCs of X-CGD patients to normal or near-normal physiological levels, exceeding the per-cell levels previously reported for CYBB cDNA transfer into X-CGD HSPCs by safe-harbor targeted insertion (21) or by lentiviral vector insertion except at high vector copy numbers (7, 8, 52). These data indicate that retention of CYBB intron 1 is sufficient to mediate physiologically normal levels of gp91^{phox} expression from the CYBB promoter, without requiring the inclusion of additional exogenous regulatory elements (such as a WPRE) in the inserted donor template, other than a strong poly-A signal such as rabbit beta-globin poly-A (45, 46). These findings highlight the utility of tailoring the design of targeting strategies and donor template constructs to include the appropriate regulatory elements necessary for optimal expression of the target gene.

Of the remaining ˜10% of X-CGD patients that are not treatable by an exon 2-13 correction strategy, approximately 4% have mutations in the upstream region including exon 1, the intron 1 splice donor site, or the CYBB promoter (1), which would require the development of alternative strategies for targeted correction of the CYBB locus to those described here, such as the targeted insertion of a donor template encompassing only the upstream region for correction of this subset of patients. Another strategy would be the incorporation of CYBB intron 1 in a complete donor template with exon 1-13 cDNA for simultaneous correction of all CYBB mutations. However, the size of intron 1 and the packaging limits of AAV complicate its inclusion in a single AAV6 donor template containing the necessary CYBB exon 1-13 cDNA, poly-A, and homologous sequences, although this approach might be enabled by the identification and inclusion of only those smaller critical sequences within intron 1 needed for physiological levels of CYBB expression, or alternatively by the inclusion of suitable exogenous enhancer elements. The remaining ˜6% of X-CGD patients have large deletions of the X-chromosome encompassing CYBB promoter and exon regions and in some cases neighboring genes (1), which would preclude targeted repair by any of these strategies, necessitating other HSPC gene therapy approaches such as CYBB cDNA transfer using a lentiviral vector containing a chimeric promoter for myeloid-specific gp91^{phox} expression (7, 8) or targeted insertion into the safe-harbor AAVS1 locus (14), as we previously described.

A number of studies have assessed chemical or molecular modulation of DNA repair pathway components to enhance HDR-mediated genome editing in human cells, including enhancement of the activity of HDR pathway components Rad51 (48, 53) and CtIP (54-56), or inhibition of NHEJ pathway components DNA ligase IV (53, 55, 57-59), Ku70/Ku80 (55, 59), DNA-PK (60-62), and 53BP1 (38, 48-50, 63, 64). However, most these studies have been conducted in immortalized cell lines or pluripotent stem cells rather than primary cells, and the effects appear to be cell type-dependent, while the majority of agents tested in CD34⁺ HSPCs have been ineffective at enhancing HDR in these cells, with only a few exhibiting efficacy (61, 62). Riesenberg and Maricic (61) reported that NU7026, a chemical inhibitor of DNA-PK, enhanced CRISPR/Cpf1-mediated HDR of an ssODN donor template in CD34⁺ HSPCs by 1.7-fold, but this resulted in a reduction of cell viability to ˜80% relative to edited controls; the effects of NU7026 could be further increased to 2.6-fold enhancement of HDR when combined with Trichostatin A and NSC 15520 to increase the activities of ATM kinase and Replication Protein A in the HDR pathway, which further reduced HSPC viability to 65%. Likewise, Jayavaradhan et al. (62) showed that NU7441, another inhibitor of DNA-PK, enhanced HDR of an AAV6 donor template in HSPCs by 2-fold, but reduced cell viability to ˜65% compared to controls. Additionally, Lomova et al. (56) reported ˜4-fold enhancement of the HDR/NHEJ ratio for an AAV6 donor template when HSPCs were pre-treated with a CDK1 inhibitor to transiently arrest cell cycle progression at S/G2 phases, combined with geminin-modified Cas9 to reduce nuclease activity during G1 phase; however, this approach predominantly decreased the incidence of NHEJ-induced indels rather than substantially increasing HDR, and cell viability following genome editing was decreased ˜50% by CDK1 inhibitor pre-treatment.

In the current study, we assessed the effect of inhibition of 53BP1, an early key regulator of DSB repair pathway choice that promotes NHEJ over HDR (39). Canny et al. (38) recently demonstrated that transient expression of mRNA encoding i53, an engineered inhibitor of 53BP1, enhances the efficiency of HDR-mediated targeted insertion of ssODNs or double-strand plasmid DNA donor templates in immortalized human cell lines; similar results were reported by others in subsequent studies (48-50) at various gene loci, also in immortalized human cells. However, the effects of i53 expression on enhancing HDR had not been reported in primary human cells or for insertion of AAV-based donor DNA templates. In our study, co-transfection of i53 mRNA at the time of electroporation with CRISPR/Cas9 RNP resulted in an approximately 1.5-fold increase in targeted insertion of AAV6 donor templates in primary HSPCs, without reducing cell viability compared to correction with Cas9 and AAV6 alone, demonstrating the benefit of 53BP1 inhibition for enhancing HDR in a clinically relevant cell type for targeted gene correction therapies. A recent study by Jayavaradhan et al. (64) tested a similar approach of inhibiting 53BP1 activity to enhance HDR in a number of immortalized human cell lines, by fusing Cas9 to a dominant-negative mutant of 53BP1 to localize its effects on NHEJ to Cas9 target sites; based on the efficacy of 53BP1 inhibition in HSPCs in our study, this Cas9 fusion might provide a further safety improvement for enhancing HDR-mediated genome editing in HSPCs, by limiting potential undesired effects of genome-wide NHEJ inhibition.

Our described strategies for CYBB exon 2-13 correction of X-CGD patient CD34⁺ HSPCs resulted in at least a partial retention of engraftment potential, as functional restoration of ROS production could be detected in phagocytes derived from HSPCs engrafted in NSG mice. However, while the overall level of human CD45⁺ cell engraftment in NSG mice was similar between the treated HSPCs and the naive X-CGD patient or healthy donor controls, the subset of successfully gene-corrected cells present within those engrafted human cells at 12 to 16 weeks post-transplant was markedly lower than was present in the treated CD34⁺ HSPC population that was initially transplanted. Similarly, reductions in gene correction following engraftment have been reported to varying degrees in a number of studies using AAV6 donor templates for HDR-mediated targeted insertion in HSPCs (19-24). This suggests that true long-term engraftable hematopoietic stem cells were corrected at a substantially lower efficiency than the overall HSPC population, or that exposure to CRISPR/Cas9 and AAV6 reduced the engraftment capacity of the subset of treated HSPCs that were successfully corrected. This latter explanation would also be consistent with the recent report by Schiroli et al. (23) that CRISPR/Cas9 and AAV6 delivery in HSPCs each induced p53-mediated activation of a DNA damage response pathway which impaired HSPC engraftment and long-term repopulation capacity; this is also consistent with the significantly reduced HSPC viability that we observed following AAV6 transduction in combination with CRISPR/Cas9 electroporation in our correction studies. Schiroli et al. also reported that transient inhibition of p53 during genome editing of HSPCs could overcome this impairment to allow a greater retention of HSPC engraftment and repopulation functions (23), which might provide a further enhancement to the X-CGD correction strategies that we describe here.

### Materials and Methods

### Human CD34⁺ HSPC Collection

Human CD34⁺ HSPCs were obtained from male healthy donors or X-CGD patients after written informed consent under the auspices of National Institute of Allergy and Infectious Diseases (NIAID) Institutional Review Board-approved protocols 05-I-0213 and 94-I-0073. The conduct of these studies conforms to the Declaration of Helsinki protocols and all United States federal regulations required for protection of human subjects. Donors underwent leukapheresis after CD34⁺ HSPC mobilization with granulocyte-colony stimulating factor (G-CSF; 15 mg/kg daily for 5 days), with some donors also receiving plerixafor at 12 hours before blood collection. After collection. CD34⁺ HSPCs were purified by CliniMACS CD34⁺ cell separation (Miltenyi Biotec; Auburn, CA) in the Cell Processing Section of the Department of Transfusion Medicine at the National Institutes of Health Clinical Center, and were cryopreserved prior to use in correction studies. X-CGD HSPCs utilized in these studies were from patients with a CYBB mutation in one of the following sites: exon 3, exon 4, exon 7, intron 10 splice donor, or exon 12.

CRISPR-Cas9 sgRNAs and AAV6 Donor Templates

Chemically modified synthetic sgRNAs targeting sites in CYBB exon 1 or exon 2 were commercially synthesized (Synthego; Menlo Park, CA or TriLink Biotechnologies; San Diego, CA) with 2′-O-methyl 3′ phosphorothioate modifications at the first 3 and last 3 nucleotides for increased RNA stability and enhanced CRISPR/Cas9 editing activity (65). The target sequence at the start of CYBB exon 1 (corresponding to CYBB1-g3 sgRNA for correction strategy 1) was CACAGCCCAGTTCCCCATGGTGG (with the protospacer adjacent motif or PAM sequence underlined); target sequences in exon 2 were TTGTGCAGCTGGTITTGGCTGGGG (corresponding to CYBB2-g8 sgRNA targeting the beginning region of exon 2 for correction strategy 2) or CCCGGTAATACCAGACAAAGAGG (corresponding to CYBB2-g3 sgRNA targeting further downstream within exon 2 for correction strategy 3).

Plasmids encoding AAV donor DNA template constructs for these correction strategies (depicted in FIG. 1) were commercially synthesized (GenScript; Piscataway, NJ or Integrated DNA Technologies; Coralville, IA) to contain a codon-optimized CYBB cDNA (36-40) (1713-bp encompassing exons 1-13 for strategy 1 or 1668-bp encompassing exons 2-13 for strategies 2 and 3) and either an ˜230-bp bGH poly-A (41) (for strategies 1 and 2) or an ˜530-bp rabbit beta-globin poly-A (45, 46) (for strategy 3). An alternative AAV donor construct for strategy 1 was designed to include an ˜600-bp WPRE for enhanced gene expression (42-44), which was also included in the construct for strategy 2. The codon-optimized CYBB cDNAs include silent mutations to the Cas9 PAM sequence and other portions of the target sequence to prevent Cas9-mediated cutting of the donor DNA template prior to or after targeted insertion. Additionally, the cDNA expression constructs in all of the donor templates are flanked on either end by ˜400-bp homology arms corresponding to sequences matching the genomic CYBB sequences on either side of the Cas9 cut site, and each vector contains ITRs for AAV packaging. AAV vectors were commercially packaged (Vigene Biosciences; Rockville, MD or SignaGen Laboratories; Frederick, MD) from these plasmid constructs using AAV6 capsid containing Y705F and Y731F tyrosine-to-phenylalanine capsid mutations for enhanced transduction of HSPCs (66), and AAV titers were determined by the manufacturers using qPCR detection of ITRs to quantify viral genome copies.

### Targeted Genome Editing in CD34⁺ HSPCs

Cryopreserved CD34⁺ HSPCs were thawed and then pre-stimulated by culturing at a density of 0.2-0.5×10⁶ cells per mL for 48-72 hours at 37° C., 5% CO₂ to induce proliferation of quiescent cells using HSPC medium consisting of StemSpan SFEM II serum-free medium (STEMCELL Technologies; Vancouver, Canada) supplemented with 0.75 μM of StemRegenin-1 (STEMCELL Technologies), 35 nM of UM171 (STEMCELL Technologies), and 100 ng/mL each of human stem cell factor, Flt3-ligand, thrombopoietin, and interleukin-6 (PeproTech; Rocky Hill, NJ). For targeted genome editing experiments. CRISPR/Cas9 RNP complexes were formed by adding 16.4 μg (100 pmol) of SpCas9 protein (Integrated DNA Technologies) and 250-500 pmol of chemically modified sgRNA to 100 μL of MaxCyte electroporation buffer (MaxCyte; Gaithersburg, MD) or P3 Nucleofector solution (Lonza; Morristown, NJ), then incubating for 10-15 minutes at room temperature. In some experiments, 5-20 μg (3.5-14 pmol) of SpCas9 mRNA (made using the mMESSAGE mMACHINE T7 ULTRA kit; Thermo Fisher Scientific; Waltham, MA) was used instead of Cas9 protein in the electroporation mixture. For studies on enhancement of HDR, 10-14 μg (48-67 pmol) of in vitro-transcribed (CellScript; Madison, WI) i53 mRNA (38) was included in the electroporation mixture for transient inhibition of 53BP1 during gene editing. Between 1-10 million HSPCs were resuspended in the electroporation mixture immediately prior to electroporation using a MaxCyte GT system with program HPSC34-3 (MaxCyte) or using a 4D-Nucleofector system with program DZ-100 (Lonza). Cells were then resuspended in HSPC medium, and AAV6 was added at an MOI of 5×10³ to 1×10⁵ viruses per cell within 20 minutes after electroporation. Cells were transduced for 18-48 hours in culture at 37° C., 5% CO₂ at a density up to 0.5×10⁶ cells per mL, followed by media change to remove residual AAV. Cell viability was assessed at 3 days after electroporation by trypan blue exclusion stain (0.4% solution; Lonza) using a hemocytometer.

### Molecular Analysis of Targeted Editing in HSPCs

Genomic DNA was extracted (by QIAGEN DNeasy Blood & Tissue kit; QIAGEN; Germantown, MD) from HSPCs at 2-5 days post-electroporation for analysis of indels and targeted donor template insertion efficiency. Indel activity of CRISPR/Cas9 (in the absence of AAV6 transduction) was determined by TIDE algorithm analysis (67) (tide.deskgen.com) or ICE analysis (68) (ice.synthego.com) of Sanger sequencing runs from high-fidelity PCRs of CYBB exon 1 or exon 2 regions. Indel analysis for CYBB1-g3 sgRNA targeting CYBB exon 1 for correction strategy 1 was performed using forward primer: 5′-tgtgactggatcattatagacc-3′ with reverse primer: 5′-aagctagaagtgagcccc-3′. Indel analysis for CYBB2-g8 sgRNA targeting CYBB exon 2 for strategy 2 was performed using forward primer: 5′-tggcctgctatcagctacc-3′ with reverse primer: 5′-actcctggatggattgctc-3′, and analysis for CYBB2-g3 sgRNA for strategy 3 was performed using forward primer: 5′-TTTAGCTGATGAGAATTCACTAGC-3′, reverse primer: 5′-TTTAAGCTAAACAATGGCACATGG-3′, and sequencing primer: 5′-ATGGGGAACAACACAG-3′. High-fidelity PCRs for indel assays were performed using Q5 High-Fidelity 2× Master Mix (New England Biolabs; Ipswich, MA) or KOD One PCR Master Mix (Toyobo; Osaka, Japan).

Molecular analysis of targeted donor template insertion was performed by ddPCR using up to 50 ng of genomic DNA that had been digested with EcoRV restriction enzyme (Thermo Fisher Scientific) to fragment genomic DNA outside of the ddPCR target amplicon and genomic reference amplicon. Samples were analyzed using a QX200 Droplet Digital PCR system and QuantaSoft Analysis Pro software version 1.0 (Bio-Rad; Hercules, CA) for detection of targeted donor template insertions in the CYBB locus using a hexachlorofluorescein-labeled probe in duplex reactions for detection of the CYBB gene on the X-chromosome as a genomic reference (22) using a 6-carboxyfluorescein-labeled probe (Integrated DNA Technologies) to quantify the frequency of HDR-mediated targeted insertion per genome. For strategy 1, detection of CYBB1-13 insertion was performed using forward primer: 5′-TCCAGCCTGTCAAAATCACA-3′, reverse primer:

5′-TACACCCGGTAGTACCACAC-3′,

and probe:

5′-CCTGGTGTGGCTGGGCCTGAACGT-3′,

for a 515-bp amplicon. For strategy 2, detection of CYBB2-13 insertion was performed using forward primer: 5′-AGCACCTGTGAGAACAGAAC-3′, reverse primer:

5′-GGTAGTACCACACGAACAGG-3′,

and probe: 5′-GTGCAGCTGGTTTGGCTCGGCCT-3′, for a 459-bp amplicon. For strategy 3, detection of CYBB2-13 insertion was performed using forward primer: 5′-TGTGGTAGAGGGAGGTGATTAG-3, reverse primer:

5′-AGCAGCTTCCGGGTATAGAA-3′,

and probe:

5′-ACTTCGGTGGGATGTCATACACGC-3′,

for a 525-bp amplicon. CYBB gene detection was performed using forward primer: 5′-GGGAAGGTAAAACTGGCAAC-3′, reverse primer: 5′-GGGCACATATACAGCTGTCT-3′, and probe: 5′-CCTCGCCAGTCTCAACAGGGACCCAGC-3′, for a 483-bp amplicon. For each CYBB ddPCR assay, forward primer sequences are only present in the genome and not in the donor template, while the underlined sequences in the probes and reverse primers are unique to the codon-optimized cDNAs present in the donor template, to ensure specific detection of targeted genomic insertions.

### In Vitro Phagocyte Differentiation

Phagocyte differentiation of HSPCs was performed by culturing cells for 12-16 days in Iscove's Modified Delbecco's Medium (Gibco; Thermo Fisher Scientific) supplemented with 10-20% fetal bovine serum (Atlanta Biologicals; R&D Systems; Minneapolis, MN) and 100 ng/mL human G-CSF (PeproTech), resulting in a mixed population of neutrophils and macrophages of varying maturation status. Phagocyte morphology was assessed by Giemsa staining of cell cytospins as previously described (59, 70). Color images of stained cells were acquired using an EVOS XL Core system (Thermo Fisher Scientific), and whole image adjustments of brightness, color balance, and contrast were performed using Adobe Photoshop software (Adobe; San Jose, CA) without additional image processing. Flow cytometry analysis of human gp91^{phox} protein expression was performed by immunostaining as previously described (70, 71). DHR flow cytometry assay of ROS production was performed as previously described (69, 70). Flow cytometry was performed using a FACSCalibur or FACSCanto system (BD Biosciences; San Jose, CA) with analysis conducted using FlowJo version 9.9.6 software for macOS (TreeStar Inc.; Ashland, OR). For comparisons of per-cell levels of gp91^{phox} protein expression and DHR activity between corrected patient cells and healthy donor control cells, MFIs of the positive gated populations in the flow cytometry analyses were compared.

### NSG Mouse Transplant of Gene Corrected Human HSPCs

The use of immunodeficient NSG mice (obtained from The Jackson Laboratory; Bar Harbor, ME) for xenotransplant studies was approved by the NIAID Institutional Animal Care and Use Committee under animal use protocol LCIM-1E. The conduct of these studies conformed to Association for Assessment and Accreditation of Laboratory Animal Care International guidelines and all U.S. federal regulations governing the protection of research animals.

Female NSG mice at 6-10 weeks of age were treated with 20 mg/kg busulfan for myelosuppressive conditioning, administered by intraperitoneal injection approximately 24 hours before transplant of 1-2 million human HSPCs via tail vein injection. For gene correction studies, X-CGD HSPCs were pre-stimulated for 48 hours prior to electroporation and AAV6 transduction, and were transplanted 2 days later. Mice received neomycin-supplemented water post-transplant for prophylaxis. At 12 weeks post-transplant, mouse peripheral blood was collected by tail venesection, lysed with ACK lysis buffer (Quality Biological, Gaithersburg, MD) for 5-7 minutes at 37° C., then stained with allophycocyanin-conjugated antibody to human CD45 pan-leukocyte marker (BD Biosciences) and co-immunostained for human gp91^{phox} protein expression as described above, for flow cytometry analysis of CD45⁺ human hematopoietic cell engraftment and gene correction in phagocytes derived from engrafted human HSPCs. Mouse bone marrow was harvested at 16 weeks post-transplant, and engrafted human CD34⁺ HSPCs were isolated using magnetic-activated cell sorting microbeads (Miltenyi Biotec) and cultured in vitro for 12-16 days in phagocyte differentiation medium to obtain mature human phagocytes for DHR analysis of functional correction as described above.

### Statistical Analysis

Statistical analyses (two-tailed paired or unpaired t-tests or one-way ANOVA) of indel frequency, targeted correction efficiency (from ddPCR or gp91^{phox} expression analyses), relative MFIs of gp91^{phox+} cells, and cell viability were performed using GraphPad Prism 8.0.1 software for macOS or 8.4.2 software for Windows (GraphPad Software, San Diego, CA).

### REFERENCES

1. Roos, D., Kuhns, D. B., Maddalena, A., Roesler, J., Lopez, J. A., Ariga, T., Avcin, T., de Boer, M., Bustamante, J., Condino-Neto, A., et al. (2010). Hematologically important mutations: X-linked chronic granulomatous disease (third update). Blood Cells Mol Dis 45: 246-265.
2. Marciano, B. E., Spalding, C., Fitzgerald, A., Mann, D., Brown, T., Osgood, S., Yockey, L., Darnell, D. N., Barnhart, L., Daub, J., et al. (2015). Common severe infections in chronic granulomatous disease. Clin Infect Dis 60: 1176-1183.
3. Stein, S., Ott, M. G., Schultze-Strasser, S., Jauch, A., Burwinkel, B., Kinner, A., Schmidt, M., Kramer, A., Schwable, J., Glimm, H., et al. (2010). Genomic instability and myelodysplasia with monosomy 7 consequent to EVI1 activation after gene therapy for chronic granulomatous disease. Nat Med 16: 198-204.
4. Ulrich, S., Anna, P., Heidi, H.-G., Elena, K., Ulrike, K., Eleonore, D. R., Canan, A., Arjan, A. v. d. L., Joachim, S., Thomas, P., et al. (2015). Successful combination of sequential gene therapy and rescue allo-HSCT in two children with X-CGD—importance of timing. Curr Gene Ther 15: 416-427.
5. Grez, M., Reichenbach, J., Schwäble, J., Seger, R., Dinauer, M. C., and Thrasher, A. J. (2011). Gene therapy of chronic granulomatous disease: the engraftment dilemma. Mol Ther 19: 28-35.
6. Yahata, T., Takanashi, T., Muguruma, Y., Ibrahim, A. A., Matsuzawa, H., Uno, T., Sheng, Y., Onizuka, M., Ito, M., Kato, S., et al. (2011). Accumulation of oxidative DNA damage restricts the self-renewal capacity of human hematopoietic stem cells. Blood 118: 2941-2950.
7. Santilli, G., Almarza, E., Brendel, C., Choi, U., Beilin, C., Blundell, M. P., Haria, S., Parsley, K. L., Kinnon, C., Malech, H. L., et al. (2011). Biochemical correction of X-CGD by a novel chimeric promoter regulating high levels of transgene expression in myeloid cells. Mol Ther 19: 122-132.
8. Kohn, D. B., Booth, C., Kang, E. M., Pai, S.-Y., Shaw, K. L., Santilli, G., Armant, M., Buckland, K. F., Choi, U., De Ravin, S. S., et al. (2020). Lentiviral gene therapy for X-linked chronic granulomatous disease. Nat Med 26: 200-206.
9. Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337: 816-821.
10. Cong, L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science 339: 819-823.
11. Mali, P., Yang, L., Esvelt, K. M., Aach, J., Guell, M., DiCarlo, J. E., Norville, J. E., and Church, G. M. (2013). RNA-guided human genome engineering via Cas9. Science 339: 823-826.
12. Umov, F. D., Miller, J. C., Lee, Y.-L., Beausejour, C. M., Rock, J. M., Augustus, S., Jamieson, A. C., Porteus, M. H., Gregory, P. D., and Holmes, M. C. (2005). Highly efficient endogenous human gene correction using designed zinc-finger nucleases. Nature 435: 646-651.
13. Moehle, E. A., Rock, J. M., Lee, Y.-L., Jouvenot, Y., DeKelver, R. C., Gregory, P. D., Umov, F. D., and Holmes, M. C. (2007). Targeted gene addition into a specified location in the human genome using designed zinc finger nucleases. Proc Natl Acad Sci USA 104: 3055-3060.
14. DeKelver, R. C., Choi, V. M., Moehle, E. A., Paschon, D. E., Hockemeyer, D., Meijsing, S. H., Sancak, Y., Cui, X., Steine, E. J., Miller, J. C., et al. (2010). Functional genomics, proteomics, and regulatory DNA analysis in isogenic settings using zinc finger nuclease-driven transgenesis into a safe harbor locus in the human genome. Genome Res 20: 1133-1142.
15. Miller, J. C., Tan, S., Qiao, G., Barlow, K. A., Wang, J., Xia, D. F., Meng, X., Paschon, D. E., Leung, E., Hinkley, S. J., et al. (2011). A TALE nuclease architecture for efficient genome editing. Nat Biotechnol 29: 143-148.
16. Levetzow, G. V., Spanholtz, J., Beckmann, J., Fischer, J., Kogler, G., Wernet, P., Punzel, M., and Giebel, B. (2006). Nucleofection, an efficient nonviral method to transfer genes into human hematopoietic stem and progenitor cells. Stem Cells Dev 15: 278-285.
17. Lesueur, L. L., Mir, L. M., and André, F. M. (2016). Overcoming the specific toxicity of large plasmids electrotransfer in primary cells in vitro. Mol Ther Nucleic Acids 5: e291-e291.
18. Sather, B. D., Romano Ibarra, G. S., Sommer, K., Curinga, G., Hale, M., Khan, I. F., Singh, S., Song, Y., Gwiazda, K., Sahni, J., et al. (2015). Efficient modification of CCR5 in primary human hematopoietic cells using a megaTAL nuclease and AAV donor template. Sci Transl Med 7: 307ra156.
19. Wang, J., Exline, C. M., DeClercq, J. J., Llewellyn, G. N., Hayward, S. B., Li, P. W.-L., Shivak, D. A., Surosky, R. T., Gregory, P. D., Holmes, M. C., et al. (2015). Homology-driven genome editing in hematopoietic stem and progenitor cells using ZFN mRNA and AAV6 donors. Nat Biotechnol 33: 1256-1263.
20. Dever, D. P., Bak, R. O., Reinisch, A., Camarena, J., Washington, G., Nicolas, C. E., Pavel-Dinu, M., Saxena, N., Wilkens, A. B., Mantri, S., et al. (2016). CRISPR/Cas9 β-globin gene targeting in human haematopoietic stem cells. Nature 539: 384-389.
21. De Ravin, S. S., Reik, A., Liu, P.-Q., Li, L., Wu, X., Su, L., Raley, C., Theobald, N., Choi, U., Song, A. H., et al. (2016). Targeted gene addition in human CD34⁺ hematopoietic cells for correction of X-linked chronic granulomatous disease. Nat Biotechnol 34: 424-429.
22. Pavel-Dinu, M., Wiebking, V., Dejene, B. T., Srifa, W., Mantri, S., Nicolas, C. E., Lee, C., Bao, G., Kildebeck, E. J., Punjya, N., et al. (2019). Gene correction for X-CGD in long-term hematopoietic stem cells. Nat Commun 10: 1634.
23. Schiroli, G., Conti, A., Ferrari, S., della Volpe, L., Jacob, A., Albano, L., Beretta. S., Calabria, A., Vavassori, V., Gasparini, P., et al. (2019). Precise gene editing preserves hematopoietic stem cell function following transient p53-mediated DNA damage response. Cell Stem Cell 24: 551-565.e558.
24. Pattabbi, S., Lot, S. N., Berger, M. P., Singh, S., Lux, C. T., Jacoby, K., Lee, C., Negre, O., Scharenberg, A. M., and Rawlings, D. J. (2019). In vivo outcome of homology-directed repair at the HBB Gene in HSC using alternative donor template delivery methods. Mol Ther Nucleic Acids 17: 277-288.
25. Gundry, Michael C., Brunetti, L., Lin, A., Mayle, Allison E., Kitano, A., Wagner, D., Hsu, Joanne I., Hoegenauer, Kevin A., Rooney, Cliona M., Goodell, Margaret A., et al. (2016). Highly efficient genome editing of murine and human hematopoietic progenitor cells by CRISPR/Cas9. Cell Reports 17: 1453-1461.
26. DeWitt, M. A., Magis, W., Bray, N. L., Wang, T., Berman, J. R., Urbinati, F., Heo, S.-J., Mitros, T., Muñoz, D. P., Boffelli, D., et al. (2016). Selection-free genome editing of the sickle mutation in human adult hematopoietic stem/progenitor cells. Sci Transl Med 8: 360ra134.
27. De Ravin, S. S., Li, L., Wu, X., Choi, U., Allen, C., Koontz, S., Lee, J., Theobald-Whiting, N., Chu, J., Garofalo, M., et al. (2017). CRISPR-Cas9 gene repair of hematopoietic stem cells from patients with X-linked chronic granulomatous disease. Sci Transl Med 9: eaah3480.
28. Antony, J. S., Latifi, N., Haque, A. K. M. A., Lamsfus-Calle, A., Daniel-Moreno, A., Graeter, S., Baskaran, P., Weinmann, P., Mezger, M., Handgretinger, R., et al. (2018). Gene correction of HBB mutations in CD34⁺ hematopoietic stem cells using Cas9 mRNA and ssODN donors. Mol Cell Pediatr 5: 9.
29. Park, S. H., Lee, C. M., Dever, D. P., Davis, T. H., Camarena, J., Srifa, W., Zhang, Y., Paikari, A., Chang, A. K., Porteus, M. H., et al. (2019). Highly efficient editing of the p-globin gene in patient-derived hematopoietic stem and progenitor cells to treat sickle cell disease. Nucleic Acids Res 47: 7955-7972.
30. Pannunzio, N. R., Watanabe, G., and Lieber, M. R. (2018). Nonhomologous DNA end-joining for repair of DNA double-strand breaks. J Biol Chem 293: 10512-10523.
31. Miyaoka, Y., Berman, J. R., Cooper, S. B., Mayerl, S. J., Chan, A. H., Zhang, B., Karlin-Neumann, G. A., and Conklin, B. R. (2016). Systematic quantification of HDR and NHEJ reveals effects of locus, nuclease, and cell type on genome-editing. Sci Rep 6: 23549.
32. Branzei, D., and Foiani, M. (2008). Regulation of DNA repair throughout the cell cycle. Nat Rev Mol Cell Biol 9: 297-308.
33. Heyer, W.-D., Ehmsen, K. T., and Liu, J. (2010). Regulation of homologous recombination in eukaryotes. Annu Rev Genet 44: 113-139.
34. Genovese, P., Schiroli, G., Escobar, G., Tomaso, T. D., Firrito, C., Calabria, A., Moi, D., Mazzieri, R., Bonini, C., Holmes, M. C., et al. (2014). Targeted genome editing in human repopulating haematopoietic stem cells. Nature 510: 235-240.
35. Hubbard, N., Hagin, D., Sommer, K., Song, Y., Khan, I., Clough, C., Ochs, H. D., Rawlings, D. J., Scharenberg, A. M., and Torgerson, T. R. (2016). Targeted gene editing restores regulated CD40L function in X-linked hyper-IgM syndrome. Blood 127: 2513-2522.
36. Sweeney, C. L., Zou, J., Choi, U., Merling, R. K., Liu, A., Bodansky, A., Burkett, S., Kim, J.-W., De Ravin, S. S., and Malech, H. L. (2017). Targeted repair of CYBB in X-CGD iPSCs requires retention of intronic sequences for expression and functional correction. Mol Ther 25: 321-330.
37. Shaul, O. (2017). How introns enhance gene expression. Int J Biochem Cell Biol 91: 145-155.
38. Canny, M. D., Moatti, N., Wan, L. C. K., Fradet-Turcotte, A., Krasner, D., Mateos-Gomez, P. A., Zimmermann, M., Orthwein, A., Juang, Y.-C., Zhang, W., et al. (2018). Inhibition of 53BP1 favors homology-dependent DNA repair and increases CRISPR-Cas9 genome-editing efficiency. Nat Biotechnol 36: 95.
39. Escribano-Díaz, C., Orthwein, A., Fradet-Turcotte, A., Xing, M., Young, Jordan T. F., Tkáč, J., Cook, Michael A., Rosebrock, Adam P., Munro, M., Canny. Marella D., et al. (2013). A cell cycle-dependent regulatory circuit composed of 53BP1-RIF1 and BRCA1-CtIP controls DNA repair pathway choice. Mol Cell 49: 872-883.
40. Moreno-Carranza, B., Gentsch, M., Stein, S., Schambach, A., Santilli, G., Rudolf, E., Ryser, M. F., Haria, S., Thrasher, A. J., Baum, C., et al. (2009). Transgene optimization significantly improves SIN vector titers, gp91^{phox} expression and reconstitution of superoxide production in X-CGD cells. Gene Ther 16: 111-118.
41. Goodwin, E. C., and Rottman, F. M. (1992). The 3′-flanking sequence of the bovine growth hormone gene contains novel elements required for efficient and accurate polyadenylation. J Biol Chem 267: 16330-16334.
42. Donello, J. E., Loeb, J. E., and Hope, T. J. (1998). Woodchuck hepatitis virus contains a tripartite posttranscriptional regulatory element. J Virol 72: 5085-5092.
43. Loeb, J. E., Cordier, W. S., Harris, M. E., Weitzman, M. D., and Hope, T. J. (1999). Enhanced expression of transgenes from adeno-associated virus vectors with the woodchuck hepatitis virus posttranscriptional regulatory element: implications for gene therapy. Hum Gene Ther 10: 2295-2305.
44. Zufferey, R., Donello, J. E., Trono, D., and Hope, T. J. (1999). Woodchuck hepatitis virus posttranscriptional regulatory element enhances expression of transgenes delivered by retroviral vectors. J Virol 73: 2886-2892.
45. Gil, A., and Proudfoot, N.J. (1987). Position-dependent sequence elements downstream of AAUAAA are required for efficient rabbit β-globin mRNA 3′ end formation. Cell 49: 399-406.
46. Lanoix, J., and Acheson, N. H. (1988). A rabbit beta-globin polyadenylation signal directs efficient termination of transcription of polyomavirus DNA. EMBO J 7: 2515-2522.
47. Marciano, B. E., Zerbe, C. S., Falcone, E. L., Ding, L., DeRavin, S. S., Daub, J., Kreuzburg, S., Yockey, L., Hunsberger, S., Foruraghi, L., et al. (2018). X-linked carriers of chronic granulomatous disease: Illness, lyonization, and stability. J Allergy Clin Immunol 141: 365-371.
48. Rees, H. A., Yeh, W.-H., and Liu, D. R. (2019). Development of hRad51-Cas9 nickase fusions that mediate HDR without double-stranded breaks. Nat Commun 10: 2212.
49. Nambiar, T. S., Billon, P., Diedenhofen, G., Hayward, S. B., Taglialatela, A., Cai, K., Huang, J.-W., Leuzzi, G., Cuella-Martin, R., Palacios, A., et al (2019). Stimulation of CRISPR-mediated homology-directed repair by an engineered RAD18 variant. Nat Commun 10: 3395.
50. Wienert, B., Nguyen, D. N., Guenther, A., Feng, S. J., Locke, M. N., Wyman, S. K., Shin, J., Kazane, K. R., Gregory, G. L., Carter, M. A. M., et al. (2020). Timed inhibition of CDC7 increases CRISPR-Cas9 mediated templated repair. Nat Commun 11: 2109.
51. Frazão, J. B., Thain, A., Zhu, Z., Luengo, M., Condino-Neto, A., and Newburger, P. E. (2015). Regulation of CYBB gene expression in human phagocytes by a distant upstream NF-κB binding site. J Cell Biochem 116: 2008-2017.
52. Roesler, J., Brenner, S., Bukovsky, A. A., Whiting-Theobald, N., Dull, T., Kelly, M., Civin, C. I., and Malech, H. L. (2002). Third-generation, self-inactivating gp91^{phox} lentivector corrects the oxidase defect in NOD/SCID mouse-repopulating peripheral blood-mobilized CD34⁺ cells from patients with X-linked chronic granulomatous disease. Blood 100: 4381-4390.
53. Pinder, J., Salsman, J., and Dellaire, G. (2015). Nuclear domain ‘knock-in’ screen for the evaluation and identification of small molecule enhancers of CRISPR-based genome editing. Nucleic Acids Res 43: 9379-9392.
54. Charpentier, M., Khedher, A. H. Y., Menoret, S., Brion, A., Lamribet, K., Dardillac, E., Boix, C., Perrouault, L., Tesson, L., Geny, S., et al. (2018). CtIP fusion to Cas9 enhances transgene integration by homology-dependent repair. Nat Commun 9: 1133.
55. Ye, L., Wang, C., Hong, L., Sun, N., Chen, D., Chen, S., and Han, F. (2018). Programmable DNA repair with CRISPRa/i enhanced homology-directed repair efficiency with a single Cas9. Cell Discov 4: 46.
56. Lomova, A., Clark, D., Campo-Fernandez, B., Flores-Bjurstróm, C., Kaufman, M., Fitz-Gibbon, S., Wang, X., Miyahira, E., Brown, D., DeWitt, M., et al. (2018). Improving gene editing outcomes in human hematopoietic stem and progenitor cells by temporal control of DNA repair. Stem Cells 37.
57. Yang, D., Scavuzzo, M. A., Chmielowiec, J., Sharp, R., Bajic, A., and Borowiak, M. (2016). Enrichment of G2/M cell cycle phase in human pluripotent stem cells enhances HDR-mediated gene repair with customizable endonucleases. Sci Rep 6: 21264.
58. Maruyama, T., Dougan, S. K., Truttmann, M. C., Bilate, A. M., Ingram, J R., and Ploegh, H. L. (2015). Increasing the efficiency of precise genome editing with CRISPR-Cas9 by inhibition of nonhomologous end joining. Nat Biotechnol 33: 538-542.
59. Chu, V. T., Weber, T., Wefers, B., Wurst, W., Sander, S., Rajewsky, K., and Kuhn, R. (2015). Increasing the efficiency of homology-directed repair for CRISPR-Cas9-induced precise gene editing in mammalian cells. Nat Biotechnol 33: 543-548.
60. Robert, F., Barbeau, M., Éthier, S., Dostie, J., and Pelletier, J. (2015). Pharmacological inhibition of DNA-PK stimulates Cas9-mediated genome editing. Genome Med 7: 93.
61. Riesenberg, S., and Maricic, T. (2018). Targeting repair pathways with small molecules increases precise genome editing in pluripotent stem cells. Nat Commun 9: 2164-2164.
62. Jayavaradhan, R., Pillis, D. M., and Malik, P. (2018). A versatile tool for the quantification of CRISPR/Cas9-induced genome editing events in human hematopoietic cell lines and hematopoietic stem/progenitor cells. J Mol Biol 431.
63. Paulsen, B. S., Mandal, P. K., Frock, R. L., Boyraz, B., Yadav, R., Upadhyayula, S., Gutierrez-Martinez, P., Ebina, W., Fasth, A., Kirchhausen, T., et al. (2017). Ectopic expression of RAD52 and dn53BP1 improves homology-directed repair during CRISPR-Cas9 genome editing. Nat Biomed Eng 1: 878-888.
64. Jayavaradhan, R., Pillis, D. M., Goodman, M., Zhang, F., Zhang, Y., Andreassen, P. R., and Malik, P. (2019). CRISPR-Cas9 fusion to dominant-negative 53BP1 enhances HDR and inhibits NHEJ specifically at Cas9 target sites. Nat Commun 10: 2866.
65. Hendel, A., Bak, R. O., Clark, J. T., Kennedy, A. B., Ryan, D. E., Roy, S., Steinfeld, I., Lunstad, B. D., Kaiser, R. J., Wilkens, A. B., et al. (2015). Chemically modified guide RNAs enhance CRISPR-Cas genome editing in human primary cells. Nat Biotechnol 33: 985.
66. Song, L., Li, X., Jayandharan, G. R., Wang, Y., Aslanidi, G. V., Ling, C., Zhong, L., Gao, G., Yoder, M. C., Ling, C., et al. (2013). High-efficiency transduction of primary human hematopoietic stem cells and erythroid lineage-restricted expression by optimized AAV6 serotype vectors in vitro and in a murine xenograft model in vivo. PLoS One 8: e58757.
67. Brinkman, E. K., Chen, T., Amendola, M., and van Steensel, B. (2014). Easy quantitative assessment of genome editing by sequence trace decomposition. Nucleic Acids Res 42: e168-e168.
68. Hsiau, T., Conant, D., Rossi, N., Maures, T., Waite, K., Yang, J., Joshi, S., Kelso, R., Holden, K., Enzmann, B. L., et al. (2019). Inference of CRISPR edits from Sanger trace data. bioRxiv: 251082.
69. Zou, J., Sweeney, C. L., Chou, B. K., Choi, U., Pan, J., Wang, H., Dowey, S. N., Cheng, L., and Malech, H. L. (2011). Oxidase-deficient neutrophils from X-linked chronic granulomatous disease iPS cells: functional correction by zinc finger nuclease-mediated safe harbor targeting. Blood 117: 5561-5572.
70. Sweeney, C. L., Merling, R. K., De Ravin, S. S., Choi, U., and Malech, H. L. (2019). Gene Editing in Chronic Granulomatous Disease. In: Knaus, UG and TL Leto (eds). NADPH Oxidases: Methods and Protocols. Springer New York: New York, NY. pp 623-665.
71. Merling, R. K., Sweeney, C. L., Chu, J., Bodansky, A., Choi, U., Priel, D. L., Kuhns, D. B., Wang, H., Vasilevsky, S., De Ravin, S. S., et al. (2015). An AAVS1-targeted minigene platform for correction of iPSCs from all five types of chronic granulomatous disease. Mol Ther 23: 147-157.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### Informal Sequence Listing

sgRNA-1-CYBB sgRNA target sequence (Exon 1)
SEQ ID NO. 1
5′-CTCTGCCACCATGGGGAACT-3′
sgRNA-2-CYBB sgRNA target sequence (Exon 1)
SEQ ID NO. 2
5′-CATTCAACCTCTGCCACCAT-3′
sgRNA-3-CYBB sgRNA target sequence (Exon 1)
SEQ ID NO: 3
5′-CACAGCCCAGTTCCCCATGG-3′
sgRNA-4-CYBB sgRNA target sequence (Exon 1)
SEQ ID NO: 4
5′-ATTCACAGCCCAGTTCCCCA-3′
sgRNA-5-CYBB sgRNA target sequence (Exon 1)
SEQ ID NO: 5
5′-GGGAACTGGGCTGTGAATGA-3′
sgRNA-6-CYBB sgRNA target sequence (Exon 1)
SEQ ID NO: 6
5′-GGAACTGGGCTGTGAATGAG-3′
sgRNA-5-CYBB sgRNA target sequence (Exon 2)
SEQ ID NO: 7
5′ gtttgtgcagCTGGTTTGGC 3
sgRNA-7-CYBB sgRNA target sequence (Exon 2)
SEQ ID NO: 8
5′ ttctgtttgtgcagCTGGTT 3′
sgRNA-8-CYBB sgRNA target sequence (Exon 2)
SEQ ID NO: 9
5′ CGACAATTCTGACGCCCAAT 3′
CYBB sgRNA target sequence (Exon 2)
SEQ ID NO: 10
5′ TTGTGCAGCTGGTTTGGCTG 3′
Codon optimized Exon 2-Exon 13 CYBB cDNA sequence
SEQ ID NO: 11
CTCGGCCTGAACGTGTTCCTGTTCGTGTGGTACTACCGGGTGTACGACATCCCC
CCCAAGTTCTTCTACACCCGGAAGCTGCTGGGCAGCGCCCTGGCCCTGGCCAGA
GCCCCTGCCGCCTGCCTGAACTTCAACTGCATGCTGATCCTGCTGCCCGTGTGC
CGGAACCTGCTGTCCTTCCTGCGGGGCAGCAGCGCCTGCTGCAGCACCAGAGT
GCGGCGGCAGCTGGACCGGAACCTGACCTTCCACAAGATGGTGGCCTGGATGA
TCGCCCTGCACAGCGCCATCCACACCATCGCCCACCTGTTCAACGTGGAGTGGT
GCGTGAACGCCCGGGTGAACAACAGCGACCCCTACAGCGTGGCCCTGAGCGAG
CTGGGCGACCGGCAGAACGAGAGCTACCTGAACTTCGCCCGGAAGCGGATCAA
GAACCCCGAGGGCGGCCTGTACCTGGCCGTGACCCTGCTGGCCGGCATCACCG
GCGTGGTGATCACCCTGTGCCTGATCCTGATCATCACCAGCAGCACCAAGACCA
TCCGGCGGAGCTACTTCGAGGTGTTCTGGTACACCCACCACCTGTTCGTGATCT
TTTTCATCGGCCTGGCCATCCACGGCGCCGAGCGGATCGTGAGGGGCCAGACC
GCCGAGAGCCTGGCCGTGCACAACATCACCGTGTGCGAGCAGAAAATCAGCGA
GTGGGGCAAGATCAAAGAGTGCCCCATCCCCCAGTTCGCCGGCAACCCCCCCA
TGACCTGGAAGTGGATCGTGGGCCCCATGTTCCTGTACCTGTGCGAGCGGCTGG
TGCGGTTCTGGCGGAGCCAGCAGAAAGTGGTGATTACCAAGGTGGTGACCCAC
CCCTTCAAGACCATCGAGCTGCAGATGAAGAAAAAGGGCTTCAAGATGGAAGT
GGGCCAGTACATCTTTGTGAAGTGCCCCAAGGTGTCCAAGCTGGAATGGCACC
CCTTCACCCTGACCAGCGCCCCTGAAGAGGACTTCTTCAGCATCCACATCAGAA
TCGTGGGCGACTGGACCGAGGGCCTGTTCAATGCCTGCGGCTGCGACAAGCAG
GAATTCCAGGACGCCTGGAAGCTGCCCAAGATCGCCGTGGACGGCCCCTTTGG
CACCGCCAGCGAGGACGTGTTCAGCTACGAGGTGGTGATGCTGGTCGGAGCCG
GCATCGGCGTGACCCCCTTCGCCAGCATCCTGAAGAGCGTGTGGTACAAGTACT
GCAACAACGCCACCAACCTGAAGCTGAAGAAGATCTACTTCTACTGGCTGTGC
CGGGACACCCACGCCTTCGAGTGGTTCGCCGATCTGCTGCAGCTGCTGGAAAG
CCAGATGCAGGAACGGAACAACGCCGGCTTCCTGAGCTACAACATCTACCTGA
CCGGCTGGGACGAGAGCCAGGCCAACCACTTCGCCGTGCACCACGACGAGGAA
AAGGACGTGATCACCGGCCTGAAGCAGAAAACCCTGTACGGCAGGCCCAACTG
GGACAACGAGTTTAAGACCATCGCCAGCCAGCACCCCAACACCCGGATCGGCG
TGTTTCTGTGCGGCCCTGAGGCCCTGGCCGAGACACTGAGCAAGCAGAGCATC
AGCAACAGCGAGAGCGGCCCCAGGGGCGTGCACTTCATCTTCAACAAAGAAAA
CTTCTGA
Construct for knocking in codon optimized CYBB cDNA into Exon 2 of the CYBB
gene to restore gene expression.
Left homology arm: 1-400 bp
CYBB cDNA: 401-2060 bp
WPRE: 2061-2650 bp
BgH Poly A: 2651-2878 bp
Right homology arm: 2879-3279 bp
SEQ ID NO: 12
agtgtcatactggtggagggaaagcaatagtaatatgttcccttcctttctcattttaagtggagtggcctgctatcagcta
cctatccaaggttaagcaaaagagaggggaaaaaaagggagtggggtaatgtaagactgataatttggtatactggccaaat
cataaaacaatcatggggaacaacacagggtggagaggttttaattatagacaagtgtagtgacactgttgaggaaagagct
ggctctaagctggttatatccagctttgtcactacctttgtcaaagttggacttggggaagtcctgacccttatctgactcc
agtcttgtgtggaatctactgtggaaatgcggaaacttcatgcaattatttctgtttgtgcagCTGGTTTGGCTCGGCCTGA
ACGTGTTCCTGTTCGTGTGGTACTACCGGGTGTACGACATCCCCCCCAAGTTCTTCTACACCCGGAAGC
TGCTGGGCAGCGCCCTGGCCCTGGCCAGAGCCCCTGCCGCCTGCCTGAACTTCA
ACTGCATGCTGATCCTGCTGCCCGTGTGCCGGAACCTGCTGTCCTTCCTGCGGG
GCAGCAGCGCCTGCTGCAGCACCAGAGTGCGGCGGCAGCTGGACCGGAACCTG
ACCTTCCACAAGATGGTGGCCTGGATGATCGCCCTGCACAGCGCCATCCACACC
ATCGCCCACCTGTTCAACGTGGAGTGGTGCGTGAACGCCCGGGTGAACAACAG
CGACCCCTACAGCGTGGCCCTGAGCGAGCTGGGCGACCGGCAGAACGAGAGCT
ACCTGAACTTCGCCCGGAAGCGGATCAAGAACCCCGAGGGCGGCCTGTACCTG
GCCGTGACCCTGCTGGCCGGCATCACCGGCGTGGTGATCACCCTGTGCCTGATC
CTGATCATCACCAGCAGCACCAAGACCATCCGGCGGAGCTACTTCGAGGTGTT
CTGGTACACCCACCACCTGTTCGTGATCTTTTTCATCGGCCTGGCCATCCACGG
CGCCGAGCGGATCGTGAGGGGCCAGACCGCCGAGAGCCTGGCCGTGCACAACA
TCACCGTGTGCGAGCAGAAAATCAGCGAGTGGGGCAAGATCAAAGAGTGCCCC
ATCCCCCAGTTCGCCGGCAACCCCCCCATGACCTGGAAGTGGATCGTGGGCCCC
ATGTTCCTGTACCTGTGCGAGCGGCTGGTGCGGTTCTGGCGGAGCCAGCAGAA
AGTGGTGATTACCAAGGTGGTGACCCACCCCTTCAAGACCATCGAGCTGCAGA
TGAAGAAAAAGGGCTTCAAGATGGAAGTGGGCCAGTACATCTTTGTGAAGTGC
CCCAAGGTGTCCAAGCTGGAATGGCACCCCTTCACCCTGACCAGCGCCCCTGA
AGAGGACTTCTTCAGCATCCACATCAGAATCGTGGGCGACTGGACCGAGGGCC
TGTTCAATGCCTGCGGCTGCGACAAGCAGGAATTCCAGGACGCCTGGAAGCTG
CCCAAGATCGCCGTGGACGGCCCCTTTGGCACCGCCAGCGAGGACGTGTTCAG
CTACGAGGTGGTGATGCTGGTCGGAGCCGGCATCGGCGTGACCCCCTTCGCCA
GCATCCTGAAGAGCGTGTGGTACAAGTACTGCAACAACGCCACCAACCTGAAG
CTGAAGAAGATCTACTTCTACTGGCTGTGCCGGGACACCCACGCCTTCGAGTGG
TTCGCCGATCTGCTGCAGCTGCTGGAAAGCCAGATGCAGGAACGGAACAACGC
CGGCTTCCTGAGCTACAACATCTACCTGACCGGCTGGGACGAGAGCCAGGCCA
ACCACTTCGCCGTGCACCACGACGAGGAAAAGGACGTGATCACCGGCCTGAAG
CAGAAAACCCTGTACGGCAGGCCCAACTGGGACAACGAGTTTAAGACCATCGC
CAGCCAGCACCCCAACACCCGGATCGGCGTGTTTCTGTGCGGCCCTGAGGCCCT
GGCCGAGACACTGAGCAAGCAGAGCATCAGCAACAGCGAGAGCGGCCCCAGG
GGCGTGCACTTCATCTTCAACAAAGAAAACTTCTGAaatcaacctctggattacaaaatttgtgaa
agattgactggtattcttaactatgttgctccttttacgctatgtggatacgctgctttaatgcctttgtatcatgctattg
cttcccgtatggctttcattttctcctccttgtataaatcctggttgctgtctctttatgaggagttgtggcccgttgtcag
gcaacgtggcgtggtgtgcactgtgtttgctgacgcaacccccactggttggggcattgccaccacctgtcagctcctttcc
gggactttcgctttccccctccctattgccacggcggaactcatcgccgcctgccttgcccgctgctggacaggggctcggc
tgttgggcactgacaattccgtggtgttgtcggggaagctgacgtcctttccatggctgctcgcctgtgttgccacctggat
tctgcgcgggacgtccttctgctacgtcccttcggccctcaatccagcggaccttccttcccgcggcctgctgccggctctg
cggcctcttccgcgtcttcgccttcgccctcagacgagtcggatctccctttgggccgcctccccgcCTCGAGagcctcgac
tgtgccttctagttgccagccatctgttgtttgcccctcccccgtgccttccttgaccctggaaggtgccactcccactgtc
ctttcctaataaaatgaggaaattgcatcgcattgtctgagtaggtgtcattctattctggggggtggggtggggcaggaca
gcaagggggaggattgggaagacaatagcaggcatgctggggatgcggtgggctTCTAGACTGGGGTTGAACGTCTTCCTCT
TTGTCTGGTATTACCGGGTTTATGATATTCCACCTAAGTTCTTTTACACAAGAAAACTTCTTGGGgtaagtataaattccat
cccatgcaatattggctggttcacatttctcatcagacattcttgttcatcttcctgttagcattttaaatacatattctga
ccaacccaaacagtaccattagcctcactccctccattgcaacttcctgggcaacagtcacttaaagccttccagtctggga
ccaacaaaagtggggcagagggccccagtggcatccttccccaaccaagaaccatgtgccattgtttagcttaaaggagaaa
tctaatactcactctgaaatggaagggcaggttggagcaatccatccagg

What is claimed is:

1. A method of genetically modifying a cell from a subject with X-linked chronic granulomatous disease (X-CGD), the method comprising:
introducing into a cell isolated from the subject a single guide RNA (sgRNA) targeting the cytochrome b-245 beta chain (CYBB) gene, an RNA-guided nuclease, and a homologous donor template comprising a CYBB cDNA comprising a nucleotide sequence having at least 80% identity to SEQ ID NO:11, flanked by a first and a second CYBB homology region; wherein:
the sgRNA binds to the nuclease and directs it to a target sequence within the CYBB gene, whereupon the nuclease cleaves the gene at the target sequence, and wherein:
the cDNA is integrated by homology directed recombination (HDR) at the site of the cleaved CYBB gene, such that the cDNA is expressed under the control of the endogenous CYBB promoter, thereby providing functional gp91^{phox} protein product in the cell.

2. The method of claim 1, wherein the method further comprises isolating the cell from the subject prior to the introducing of the sgRNA, RNA-guided nuclease, and homologous donor template.

3. The method of claim 1 or 2, wherein the target sequence of the sgRNA is within exon 1 of the CYBB gene, and wherein the CYBB cDNA comprises exons 1-13 of the CYBB gene.

4. The method of claim 1 or 2, wherein the target sequence of the sgRNA is within exon 2 of the CYBB gene, and wherein the CYBB cDNA comprises exons 2-13 of the CYBB gene.

5. The method of claim 3, wherein the sgRNA comprises a nucleotide sequence complementary to a sequence selected from the group consisting of SEQ ID NO:1, SEQ ID NO:2, SEQ ID NO:3, SEQ ID NO:4, SEQ ID NO:5, and SEQ ID NO:6.

6. The method of claim 5, wherein the sgRNA comprises a nucleotide sequence complementary to SEQ ID NO: 3.

7. The method of claim 4, wherein the sgRNA comprises a nucleotide sequence complementary to a sequence selected from the group consisting of SEQ ID NO:7, SEQ ID NO:8, SEQ ID NO:9, and SEQ ID NO:10.

8. The method of claim 7, wherein the sgRNA comprises a nucleotide sequence complementary to SEQ ID NO:9.

9. The method of any one of claims 1 to 8, wherein the sgRNA comprises 2′-O-methyl-3′-phosphorothioate (MS) modifications at one or more nucleotides.

10. The method of claim 9, wherein the 2′-O-methyl-3′-phosphorothioate (MS) modifications are present at the three terminal nucleotides of the 5′ and 3′ ends.

11. The method of any one of claims 1 to 10, wherein the RNA-guided nuclease is Cas9.

12. The method of any one of claims 1 to 11, wherein the sgRNA and the RNA-guided nuclease are introduced into the cell as a ribonucleoprotein (RNP).

13. The method of claim 12, wherein the RNP is introduced into the cell by electroporation.

14. The method of any one of claims 1 to 13, further comprising introducing i53 into the cell.

15. The method of claim 14, wherein the i53 is introduced by introducing an i53 mRNA.

16. The method of claim 15, wherein the mRNA is introduced by electroporation together with the RNP.

17. The method of any one of claims 1 to 16, wherein the CYBB cDNA comprises a nucleotide sequence having at least 85%, 90%, 95%, 96%, 97%, 98%, 99% or more identity to SEQ ID NO:11.

18. The method of claim 17, wherein the CYBB cDNA comprises the nucleotide sequence of SEQ ID NO: 11.

19. The method of any one of claims 1 to 18, wherein the homologous donor template further comprises a polyadenylation signal at the 3′ end of the cDNA, wherein both the cDNA and the polyadenylation signal are flanked by the first and the second CYBB homology regions on the template.

20. The method of claim 19, wherein the polyadenylation signal is a bovine growth hormone polyadenylation signal or rabbit beta-globin polyadenylation signal.

21. The method of any one of claims 1 to 20, wherein the first and/or second CYBB homology region comprises nucleotides 1-400 or 2879-3279 of SEQ ID NO:12, or a contiguous portion of nucleotides 1-400 or 2879-3279 of SEQ ID NO:12.

22. The method of claim 21, wherein the first and second CYBB homology regions comprise nucleotides 1-400 or 2879-3279 of SEQ ID NO:12.

23. The method of any one of claims 1 to 22, wherein the homologous template further comprises a woodchuck hepatitis virus post-transcriptional regulatory element (WPRE).

24. The method of any one of claims 1 to 23, wherein the homologous donor template comprises the sequence of SEQ ID NO:12.

25. The method of any one of claims 1 to 24, wherein the homologous donor template is introduced into the cells using a recombinant adeno-associated virus (rAAV) serotype 6 vector.

26. The method of any one of claims 1 to 25, wherein the homologous donor template further comprises a selectable marker.

27. The method of any of claims 1 to 26, wherein the cell is a CD34⁺ hematopoietic stem and progenitor cell (HSPC).

28. The method of claim 27, wherein the CD34⁺ HSPC is isolated from the peripheral blood.

29. The method of any one of claims 1 to 28, wherein expression of the integrated CYBB cDNA provides a level of functional gp91^{phox} protein product in the cell that is at least 70%, 80%, 90%, or more of the level in a healthy control cell.

30. The method of any one of claims 1 to 29, wherein expression of the integrated CYBB cDNA leads to a level of reactive oxygen species (ROS) production in the cell that is at least 80%, 90%, 100%, or more of the level in a healthy control cell as measured by the mean fluorescence intensity (MFI) of positive gated cells by dihydrorhodamine-123 (DHR) assay.

31. A method of treating a subject with X-CGD, comprising (i) genetically modifying a cell from the subject using the method of any one of claims 1 to 30, and (ii) reintroducing the cell into the subject.

32. The method of claim 31, wherein the cell is reintroduced into the subject by systemic transplantation.

33. The method of claim 32, wherein the systemic transplantation comprises intravenous administration.

34. The method of claim 31, wherein the cell is reintroduced into the subject by local transplantation.

35. The method of claim 34, wherein the local transplantation comprises intrafemoral or intrahepatic administration.

36. The method of any one of claims 31 to 35, wherein the cell is cultured and/or selected prior to being reintroduced into the subject.

37. An sgRNA that specifically targets exon 1 of the CYBB gene, wherein the sgRNA comprises a nucleotide sequence complementary to the sequence of any one of SEQ ID NOS:1-6.

38. An sgRNA that specifically targets exon 2 of the CYBB gene, wherein the sgRNA comprises a nucleotide sequence complementary to the sequence of any one of SEQ ID NOS:7-10.

39. The sgRNA of claim 37 or 38, wherein the sgRNA comprises 2′-O-methyl-3′-phosphorothioate (MS) modifications at one or more nucleotides.

40. The sgRNA of claim 39, wherein the 2′-O-methyl-3′-phosphorothioate (MS) modifications are present at the three terminal nucleotides of the 5′ and 3′ ends.

41. A homologous donor template comprising:
(i) a CYBB cDNA comprising a nucleotide sequence comprising at least 80% identity to SEQ ID NO:11;
(ii) a first CYBB homology region located to one side of the cDNA within the donor template; and
(iii) a second CYBB homology region located to the other side of the cDNA within the donor template.

42. The donor template of claim 41, wherein the first CYBB homology region comprises nucleotides 1-400 of SEQ ID NO:12, or a contiguous portion thereof, and the second CYBB homology region comprises nucleotides 2879-3279 of SEQ ID NO:12, or a contiguous portion thereof.

43. The donor template of claim 41 or 42, wherein the CYBB cDNA comprises exons 1-13 of the CYBB gene.

44. The donor template of claim 41 or 42, wherein the CYBB cDNA comprises exons 2-13 of the CYBB gene.

45. The donor template of claim 41 or 42, wherein the CYBB cDNA comprises a nucleotide sequence having at least 85%, 90%, 95%, 96%, 97%, 98%, 99% or more identity to SEQ ID NO:11.

46. The donor template of any one of claims 41 to 45, wherein the CYBB cDNA is codon optimized.

47. The donor template of claim 45, wherein the CYBB cDNA comprises the nucleotide sequence of SEQ ID NO: 11.

48. The donor template of any one of claims 41 to 47, further comprising a polyadenylation signal at the 3′ end of the CYBB cDNA, wherein both the cDNA and the polyadenylation signal are flanked by the first and second CYBB homology regions on the template.

49. The donor template of claim 48, wherein the polyadenylation signal is a bovine growth hormone polyadenylation signal or rabbit beta-globin polyadenylation signal.

50. The donor template of claim 49, wherein the template comprises the sequence of SEQ ID NO: 12.

51. The donor template of any one of claims 41 to 50, further comprising a selectable marker.

52. The donor template of any one of claims 41 to 51, further comprising a woodchuck hepatitis virus post-transcriptional regulatory element (WPRE).

53. An isolated HSPC comprising the sgRNA of any one of claims 37 to 40, or a homologous donor template of any one of claims 41 to 52.

54. An isolated, genetically modified HSPC comprising an exogenous, codon-optimized CYBB cDNA integrated at the endogenous CYBB gene, wherein the integrated cDNA comprises a nucleotide sequence having at least 80% identity to SEQ ID NO:11.

55. The HSPC of claim 54, wherein the CYBB cDNA comprises a nucleotide sequence having at least 85%, 90%, 95%, 96%, 97%, 98%, 99% or more identity to SEQ ID NO:11.

56. The HSPC of claim 55, wherein the CYBB cDNA comprises the nucleotide sequence of SEQ ID NO:11.

57. The HSPC of any one of claims 54 to 56, wherein the exogenous CYBB cDNA comprises exons 1-13, and wherein the cDNA is integrated within exon 1 of the endogenous CYBB gene.

58. The HSPC of any one of claims 54 to 56, wherein the exogenous CYBB cDNA comprises exons 2-13, and wherein the cDNA is integrated within exon 2 of the endogenous CYBB gene.

59. The HSPC of any one of claims 54 to 56, wherein the HSPC was modified using the method of any one of claims 1 to 36.

60. A pharmaceutical composition comprising a plurality of genetically modified HSPCs comprising an exogenous, codon-optimized CYBB cDNA integrated at exon 2 of the endogenous CYBB gene, wherein the integrated cDNA comprises a nucleotide sequence having at least 80% identity to SEQ ID NO:11.

61. The pharmaceutical composition of claim 60, wherein the composition further comprises non-genetically modified HSPC and/or HSPC comprising INDELS at the CYBB locus.

62. The pharmaceutical composition of claim 61, wherein the composition is comprised of at least 5% of genetically modified HSPC comprising the integrated CYBB cDNA.

63. The pharmaceutical composition of claim 61, wherein the composition is comprised of at least 20% of genetically modified HSPC comprising the integrated CYBB cDNA.


SEQUENCE LISTING (66 sequences)

SEQ ID NO: 1 - DNA, 20 bp, Artificial Sequence
  source: /note=“Description of Artificial Sequence Synthetic oligonucleotide”
ctctgccaccatggggaact

SEQ ID NO: 2 - DNA, 20 bp, Artificial Sequence
  source: /note=“Description of Artificial Sequence Synthetic oligonucleotide”
cattcaacctctgccaccat

SEQ ID NO: 3 - DNA, 20 bp, Artificial Sequence
  source: /note=“Description of Artificial Sequence Synthetic oligonucleotide”
cacagcccagttccccatgg

SEQ ID NO: 4 - DNA, 20 bp, Artificial Sequence
  source: /note=“Description of Artificial Sequence Synthetic oligonucleotide”
attcacagcccagttcccca

SEQ ID NO: 5 - DNA, 20 bp, Artificial Sequence
  source: /note=“Description of Artificial Sequence Synthetic oligonucleotide”
gggaactgggctgtgaatga

SEQ ID NO: 6 - DNA, 20 bp, Artificial Sequence
  source: /note=“Description of Artificial Sequence Synthetic oligonucleotide”
ggaactgggctgtgaatgag

SEQ ID NO: 7 - DNA, 20 bp, Artificial Sequence
  source: /note=“Description of Artificial Sequence Synthetic oligonucleotide”
gtttgtgcagctggtttggc

SEQ ID NO: 8 - DNA, 20 bp, Artificial Sequence
  source: /note=“Description of Artificial Sequence Synthetic oligonucleotide”
ttctgtttgtgcagctggtt

SEQ ID NO: 9 - DNA, 20 bp, Artificial Sequence
  source: /note=“Description of Artificial Sequence Synthetic oligonucleotide”
cgacaattctgacgcccaat

SEQ ID NO: 10 - DNA, 20 bp, Artificial Sequence
  source: /note=“Description of Artificial Sequence Synthetic oligonucleotide”
ttgtgcagctggtttggctg

SEQ ID NO: 11 - DNA, 1659 bp, Artificial Sequence
  source: /note=“Description of Artificial Sequence Synthetic polynucleotide”
ctcggcctgaacgtgttcctgttcgtgtggtactaccgggtgtacgacatcccccccaagttcttctacacccggaagctgctgggcagcgccctggccctggccagagcccctgccgcctgcctgaacttcaactgcatgctgatcctgctgcccgtgtgccggaacctgctgtccttcctgcggggcagcagcgcctgctgcagcaccagagtgcggcggcagctggaccggaacctgaccttccacaagatggtggcctggatgatcgccctgcacagcgccatccacaccatcgcccacctgttcaacgtggagtggtgcgtgaacgcccgggtgaacaacagcgacccctacagcgtggccctgagcgagctgggcgaccggcagaacgagagctacctgaacttcgcccggaagcggatcaagaaccccgagggcggcctgtacctggccgtgaccctgctggccggcatcaccggcgtggtgatcaccctgtgcctgatcctgatcatcaccagcagcaccaagaccatccggcggagctacttcgaggtgttctggtacacccaccacctgttcgtgatctttttcatcggcctggccatccacggcgccgagcggatcgtgaggggccagaccgccgagagcctggccgtgcacaacatcaccgtgtgcgagcagaaaatcagcgagtggggcaagatcaaagagtgccccatcccccagttcgccggcaacccccccatgacctggaagtggatcgtgggccccatgttcctgtacctgtgcgagcggctggtgcggttctggcggagccagcagaaagtggtgattaccaaggtggtgacccaccccttcaagaccatcgagctgcagatgaagaaaaagggcttcaagatggaagtgggccagtacatctttgtgaagtgccccaaggtgtccaagctggaatggcaccccttcaccctgaccagcgcccctgaagaggacttcttcagcatccacatcagaatcgtgggcgactggaccgagggcctgttcaatgcctgcggctgcgacaagcaggaattccaggacgcctggaagctgcccaagatcgccgtggacggcccctttggcaccgccagcgaggacgtgttcagctacgaggtggtgatgctggtcggagccggcatcggcgtgacccccttcgccagcatcctgaagagcgtgtggtacaagtactgcaacaacgccaccaacctgaagctgaagaagatctacttctactggctgtgccgggacacccacgccttcgagtggttcgccgatctgctgcagctgctggaaagccagatgcaggaacggaacaacgccggcttcctgagctacaacatctacctgaccggctgggacgagagccaggccaaccacttcgccgtgcaccacgacgaggaaaaggacgtgatcaccggcctgaagcagaaaaccctgtacggcaggcccaactgggacaacgagtttaagaccatcgccagccagcaccccaacacccggatcggcgtgtttctgtgcggccctgaggccctggccgagacactgagcaagcagagcatcagcaacagcgagagcggccccaggggcgtgcacttcatcttcaacaaagaaaacttctga

SEQ ID NO: 12 - DNA, 3287 bp, Artificial Sequence
  source: /note=“Description of Artificial Sequence Synthetic polynucleotide”
agtgtcatactggtggagggaaagcaatagtaatatgttcccttcctttctcattttaagtggagtggcctgctatcagctacctatccaaggttaagcaaaagagaggggaaaaaaagggagtggggtaatgtaagactgataatttggtatactggccaaatcataaaacaatcatggggaacaacacagggtggagaggttttaattatagacaagtgtagtgacactgttgaggaaagagctggctctaagctggttatatccagctttgtcactacctttgtcaaagttggacttggggaagtcctgacccttatctgactccagtcttgtgtggaatctactgtggaaatgcggaaacttcatgcaattatttctgtttgtgcagctggtttggctcggcctgaacgtgttcctgttcgtgtggtactaccgggtgtacgacatcccccccaagttcttctacacccggaagctgctgggcagcgccctggccctggccagagcccctgccgcctgcctgaacttcaactgcatgctgatcctgctgcccgtgtgccggaacctgctgtccttcctgcggggcagcagcgcctgctgcagcaccagagtgcggcggcagctggaccggaacctgaccttccacaagatggtggcctggatgatcgccctgcacagcgccatccacaccatcgcccacctgttcaacgtggagtggtgcgtgaacgcccgggtgaacaacagcgacccctacagcgtggccctgagcgagctgggcgaccggcagaacgagagctacctgaacttcgcccggaagcggatcaagaaccccgagggcggcctgtacctggccgtgaccctgctggccggcatcaccggcgtggtgatcaccctgtgcctgatcctgatcatcaccagcagcaccaagaccatccggcggagctacttcgaggtgttctggtacacccaccacctgttcgtgatctttttcatcggcctggccatccacggcgccgagcggatcgtgaggggccagaccgccgagagcctggccgtgcacaacatcaccgtgtgcgagcagaaaatcagcgagtggggcaagatcaaagagtgccccatcccccagttcgccggcaacccccccatgacctggaagtggatcgtgggccccatgttcctgtacctgtgcgagcggctggtgcggttctggcggagccagcagaaagtggtgattaccaaggtggtgacccaccccttcaagaccatcgagctgcagatgaagaaaaagggcttcaagatggaagtgggccagtacatctttgtgaagtgccccaaggtgtccaagctggaatggcaccccttcaccctgaccagcgcccctgaagaggacttcttcagcatccacatcagaatcgtgggcgactggaccgagggcctgttcaatgcctgcggctgcgacaagcaggaattccaggacgcctggaagctgcccaagatcgccgtggacggcccctttggcaccgccagcgaggacgtgttcagctacgaggtggtgatgctggtcggagccggcatcggcgtgacccccttcgccagcatcctgaagagcgtgtggtacaagtactgcaacaacgccaccaacctgaagctgaagaagatctacttctactggctgtgccgggacacccacgccttcgagtggttcgccgatctgctgcagctgctggaaagccagatgcaggaacggaacaacgccggcttcctgagctacaacatctacctgaccggctgggacgagagccaggccaaccacttcgccgtgcaccacgacgaggaaaaggacgtgatcaccggcctgaagcagaaaaccctgtacggcaggcccaactgggacaacgagtttaagaccatcgccagccagcaccccaacacccggatcggcgtgtttctgtgcggccctgaggccctggccgagacactgagcaagcagagcatcagcaacagcgagagcggccccaggggcgtgcacttcatcttcaacaaagaaaacttctgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctcgagagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggcttctagactggggttgaacgtcttcctctttgtctggtattaccgggtttatgatattccacctaagttcttttacacaagaaaacttcttggggtaagtataaattccatcccatgcaatattggctggttcacatttctcatcagacattcttgttcatcttcctgttagcattttaaatacatattctgaccaacccaaacagtaccattagcctcactccctccattgcaacttcctgggcaacagtcacttaaagccttccagtctgggaccaacaaaagtggggcagagggccccagtggcatccttccccaaccaagaaccatgtgccattgtttagcttaaaggagaaatctaatactcactctgaaatggaagggcaggttggagcaatccatccagg

SEQ ID NO: 13 - DNA, 23 bp, Artificial Sequence
  source: /note=“Description of Artificial Sequence Synthetic oligonucleotide”
cacagcccagttccccatggtgg

SEQ ID NO: 14 - DNA, 23 bp, Artificial Sequence
  source: /note=“Description of Artificial Sequence Synthetic oligonucleotide”
ttgtgcagctggtttggctgggg

SEQ ID NO: 15 - DNA, 23 bp, Artificial Sequence
  source: /note=“Description of Artificial Sequence Synthetic oligonucleotide”
cccggtaataccagacaaagagg

SEQ ID NO: 16 - DNA, 24 bp, Artificial Sequence
  source: /note=“Description of Artificial Sequence Synthetic primer”
tttagctgatgagaattcactagc

SEQ ID NO: 17 - DNA, 24 bp, Artificial Sequence
  source: /note=“Description of Artificial Sequence Synthetic primer”
tttaagctaaacaatggcacatgg

SEQ ID NO: 18 - DNA, 16 bp, Artificial Sequence
  source: /note=“Description of Artificial Sequence Synthetic primer”
atggggaacaacacag

SEQ ID NO: 19 - DNA, 20 bp, Artificial Sequence
  source: /note=“Description of Artificial Sequence Synthetic primer”
tccagcctgtcaaaatcaca

SEQ ID NO: 20 - DNA, 20 bp, Artificial Sequence
  source: /note=“Description of Artificial Sequence Synthetic primer”
tacacccggtagtaccacac

SEQ ID NO: 21 - DNA, 24 bp, Artificial Sequence
  source: /note=“Description of Artificial Sequence Synthetic probe”
cctggtgtggctgggcctgaacgt

SEQ ID NO: 22 - DNA, 20 bp, Artificial Sequence
  source: /note=“Description of Artificial Sequence Synthetic primer”
agcacctgtgagaacagaac

SEQ ID NO: 23 - DNA, 20 bp, Artificial Sequence
  source: /note=“Description of Artificial Sequence Synthetic primer”
ggtagtaccacacgaacagg

SEQ ID NO: 24 - DNA, 23 bp, Artificial Sequence
  source: /note=“Description of Artificial Sequence Synthetic probe”
gtgcagctggtttggctcggcct

SEQ ID NO: 25 - DNA, 22 bp, Artificial Sequence
  source: /note=“Description of Artificial Sequence Synthetic primer”
tgtggtagagggaggtgattag

SEQ ID NO: 26 - DNA, 20 bp, Artificial Sequence
  source: /note=“Description of Artificial Sequence Synthetic primer”
agcagcttccgggtatagaa

SEQ ID NO: 27 - DNA, 24 bp, Artificial Sequence
  source: /note=“Description of Artificial Sequence Synthetic probe”
acttcggtgggatgtcatacacgc

SEQ ID NO: 28 - DNA, 20 bp, Artificial Sequence
  source: /note=“Description of Artificial Sequence Synthetic primer”
gggaaggtaaaactggcaac

SEQ ID NO: 29 - DNA, 20 bp, Artificial Sequence
  source: /note=“Description of Artificial Sequence Synthetic primer”
gggcacatatacagctgtct

SEQ ID NO: 30 - DNA, 27 bp, Artificial Sequence
  source: /note=“Description of Artificial Sequence Synthetic probe”
cctcgccagtctcaacagggacccagc

SEQ ID NO: 31 - DNA, 12 bp, Artificial Sequence
  source: /note=“Description of Artificial Sequence Synthetic oligonucleotide”
ccaccatgggga

SEQ ID NO: 32 - DNA, 11 bp, Artificial Sequence
  source: /note=“Description of Artificial Sequence Synthetic oligonucleotide”
tttggctgggg

SEQ ID NO: 33 - DNA, 11 bp, Artificial Sequence
  source: /note=“Description of Artificial Sequence Synthetic oligonucleotide”
cctctttgtct

SEQ ID NO: 34 - DNA, 40 bp, Artificial Sequence
  source: /note=“Description of Artificial Sequence Synthetic oligonucleotide”
acattcaacctctgccaccatggggaactgggctgtgaat

SEQ ID NO: 35 - DNA, 41 bp, Artificial Sequence
  source: /note=“Description of Artificial Sequence Synthetic oligonucleotide”
  modified_base (21)..(21): a, c, t, g, unknown or other
acattcaacctctgccaccantggggaactgggctgtgaat

SEQ ID NO: 36 - DNA, 39 bp, Artificial Sequence
  source: /note=“Description of Artificial Sequence Synthetic oligonucleotide”
acattcaacctctgccaccaggggaactgggctgtgaat

SEQ ID NO: 37 - DNA, 30 bp, Artificial Sequence
  source: /note=“Description of Artificial Sequence Synthetic oligonucleotide”
acattcaacctctgccaccgggctgtgaat

SEQ ID NO: 38 - DNA, 32 bp, Artificial Sequence
  source: /note=“Description of Artificial Sequence Synthetic oligonucleotide”
acattcaacctctgccaccctgggctgtgaat

SEQ ID NO: 39 - DNA, 24 bp, Artificial Sequence
  source: /note=“Description of Artificial Sequence Synthetic oligonucleotide”
acattcaacctctgccacctgaat

SEQ ID NO: 40 - DNA, 36 bp, Artificial Sequence
  source: /note=“Description of Artificial Sequence Synthetic oligonucleotide”
acattcaacctctgccaccggaactgggctgtgaat

SEQ ID NO: 41 - DNA, 30 bp, Artificial Sequence
  source: /note=“Description of Artificial Sequence Synthetic oligonucleotide”
acattcaacctctgccactgggctgtgaat

SEQ ID NO: 42 - DNA, 39 bp, Artificial Sequence
  source: /note=“Description of Artificial Sequence Synthetic oligonucleotide”
acattcaacctctgccacctggggaactgggctgtgaat

SEQ ID NO: 43 - DNA, 42 bp, Artificial Sequence
  source: /note=“Description of Artificial Sequence Synthetic oligonucleotide”
  modified_base (21)..(22): a, c, t, g, unknown or other
acattcaacctctgccaccanntggggaactgggctgtgaat

SEQ ID NO: 44 - DNA, 33 bp, Artificial Sequence
  source: /note=“Description of Artificial Sequence Synthetic oligonucleotide”
acattcaacctctgccaccactgggctgtgaat

SEQ ID NO: 45 - DNA, 31 bp, Artificial Sequence
  source: /note=“Description of Artificial Sequence Synthetic oligonucleotide”
acattcaacctctgccaccagggctgtgaat

SEQ ID NO: 46 - DNA, 38 bp, Artificial Sequence
  source: /note=“Description of Artificial Sequence Synthetic oligonucleotide”
acattcaacctctgccactggggaactgggctgtgaat

SEQ ID NO: 47 - DNA, 22 bp, Artificial Sequence
  source: /note=“Description of Artificial Sequence Synthetic oligonucleotide”
acattcaacctctgccgtgaat

SEQ ID NO: 48 - DNA, 40 bp, Artificial Sequence
  source: /note=“Description of Artificial Sequence Synthetic oligonucleotide”
tgtttgtgcagctggtttggctggggttgaacgtcttcct

SEQ ID NO: 49 - DNA, 31 bp, Artificial Sequence
  source: /note=“Description of Artificial Sequence Synthetic oligonucleotide”
tgtttgtgcagctggggttgaacgtcttcct

SEQ ID NO: 50 - DNA, 36 bp, Artificial Sequence
  source: /note=“Description of Artificial Sequence Synthetic oligonucleotide”
tgtttgtgcagctggtttggggttgaacgtcttcct

SEQ ID NO: 51 - DNA, 31 bp, Artificial Sequence
  source: /note=“Description of Artificial Sequence Synthetic oligonucleotide”
tgtttgtgcagctggtttggaacgtcttcct

SEQ ID NO: 52 - DNA, 31 bp, Artificial Sequence
  source: /note=“Description of Artificial Sequence Synthetic oligonucleotide”
tgtttgtgcagctggtgttgaacgtcttcct

SEQ ID NO: 53 - DNA, 30 bp, Artificial Sequence
  source: /note=“Description of Artificial Sequence Synthetic oligonucleotide”
tgtttgtgcagctgggttgaacgtcttcct

SEQ ID NO: 54 - DNA, 39 bp, Artificial Sequence
  source: /note=“Description of Artificial Sequence Synthetic oligonucleotide”
tgtttgtgcagctggtttggtggggttgaacgtcttcct

SEQ ID NO: 55 - DNA, 21 bp, Artificial Sequence
  source: /note=“Description of Artificial Sequence Synthetic oligonucleotide”
tgtttgtgcagctggtttcct

SEQ ID NO: 56 - DNA, 41 bp, Artificial Sequence
  source: /note=“Description of Artificial Sequence Synthetic oligonucleotide”
  modified_base (21)..(21): a, c, t, g, unknown or other
tgtttgtgcagctggtttggnctggggttgaacgtcttcct

SEQ ID NO: 57 - DNA, 40 bp, Artificial Sequence
  source: /note=“Description of Artificial Sequence Synthetic oligonucleotide”
gggttgaacgtcttcctctttgtctggtattaccgggttt

SEQ ID NO: 58 - DNA, 34 bp, Artificial Sequence
  source: /note=“Description of Artificial Sequence Synthetic oligonucleotide”
gggttgaacgtcttcctctggtattaccgggttt

SEQ ID NO: 59 - DNA, 41 bp, Artificial Sequence
  source: /note=“Description of Artificial Sequence Synthetic oligonucleotide”
  modified_base (21)..(21): a, c, t, g, unknown or other
gggttgaacgtcttcctcttntgtctggtattaccgggttt

SEQ ID NO: 60 - DNA, 28 bp, Artificial Sequence
  source: /note=“Description of Artificial Sequence Synthetic oligonucleotide”
gggttgaacgtcttcctcttccgggttt

SEQ ID NO: 61 - DNA, 28 bp, Artificial Sequence
  source: /note=“Description of Artificial Sequence Synthetic oligonucleotide”
gggttgaacgtcttccattaccgggttt

SEQ ID NO: 62 - DNA, 28 bp, Artificial Sequence
  source: /note=“Description of Artificial Sequence Synthetic oligonucleotide”
gggttgaacgtctggtattaccgggttt

SEQ ID NO: 63 - DNA, 22 bp, Artificial Sequence
  source: /note=“Description of Artificial Sequence Synthetic primer”
tgtgactggatcattatagacc

SEQ ID NO: 64 - DNA, 18 bp, Artificial Sequence
  source: /note=“Description of Artificial Sequence Synthetic primer”
aagctagaagtgagcccc

SEQ ID NO: 65 - DNA, 19 bp, Artificial Sequence
  source: /note=“Description of Artificial Sequence Synthetic primer”
tggcctgctatcagctacc

SEQ ID NO: 66 - DNA, 19 bp, Artificial Sequence
  source: /note=“Description of Artificial Sequence Synthetic primer”
actcctggatggattgctc